(19) United States
(12) Patent Application Publication
(10) Pub. No.: US 2023/0295565 A1
(43) Pub. Date: Sep. 21, 2023

(54) UNIVERSAL ANTIGEN-SPECIFIC T CELL BANKS AND METHODS OF MAKING AND USING THE SAME THERAPEUTICALLY

(71) Applicant: Baylor College of Medicine, Houston, TX (US)

(72) Inventors: Ann Marie Leen, Houston, TX (US); Juan Fernando Vera Valdes, Houston, TX (US)

(21) Appl. No.: 18/018,552

(22) PCT Filed: Feb. 2, 2021
(86) PCT No.: PCT/US2021/016266

§ 371 Date: Jan. 28, 2023

Related U.S. Application Data
(63) Continuation-in-part of application No. PCT/US2020/044080, filed on Jul. 29, 2020.

(51) Int. Cl. C12N 5/0783; A61K 35/17; A61P 31/12

(52) U.S. Cl. CPC C12N 5/0636; A61K 35/17; A61P 31/12; C12N 2501/2304; C12N 2501/2307

(57) ABSTRACT

Embodiments of the disclosure include universal antigen-specific T cell compositions, and methods of making and using the same. Embodiments of the disclosure also include methods of identifying and selecting suitable donors for use in constructing donor minibanks of antigen-specific T cell lines; donor minibanks of antigen-specific T cell lines; universal antigen-specific T cell compositions comprising a plurality of the antigen specific T cell lines from such donor minibanks, and donor banks made up of a plurality of such minibanks. The present disclosure includes methods of treating a disease or condition comprising administering to a patient at least one universal antigen-specific T cell composition disclosed herein.

### CROSS-REFERENCE TO RELATED APPLICATIONS

This application claims priority to International Patent Cooperation Treaty Application No. PCT/US2020/044080, filed Jul. 29, 2020, which is incorporated by reference herein in its entirety.

### FIELD

Embodiments of the disclosure concern at least the fields of cell biology, molecular biology, immunology, and medicine.

### BACKGROUND

Viral infections are a serious cause of morbidity and mortality after allogeneic hematopoietic stem cell transplantation (allo-HSCT), which is the treatment of choice for a variety of disorders. Post-transplant, however, graft versus host disease (GVHD), primary disease relapse and viral infections remain major causes of morbidity and mortality. Infections associated with viral pathogens include, but are not limited to CMV, BK virus (BKV), and adenovirus (AdV). Viral infections are detected in the majority of allograft recipients. Although available for some viruses, antiviral drugs are not always effective, highlighting the need for novel therapies. One strategy to treat these viral infections is with adoptive immunotherapy, e.g., adoptive T cell transfer, including at least infusion of donor-derived virus-specific T cells. With this approach, one can extract cells from a donor, expand virus-specific populations ex vivo and, finally, infuse the T cell product into the stem cell transplant recipient. Similar approaches may be taken to treat cancers with adoptively transferred T cells with specificity for tumor associated antigen.

Adoptive immunotherapy involves implanting or infusing disease-specific and/or engineered cells such as T cells, (e.g., antigen-specific T cells) and chimeric antigen receptor (CAR)-expressing T cells), into individuals with the aim of recognizing, targeting, and destroying disease-associated cells. Adoptive immunotherapies have become a promising approach for the treatment of many diseases and disorders, including cancer, post-transplant lymphoproliferative disorders, infectious diseases (e.g., viral and other pathogenic infections), and autoimmune diseases. For example, in vitro expanded donor-derived and third-party virus-specific T cells targeting Adv, EBV, CMV, BK, HHV6 have shown to be safe when adoptively transferred to stem cell transplant patients with viral infections. Virus-specific T cells reconstituted antiviral immunity for Adv, EBV, CMV, BK and HHV6, were effective in clearing disease, and exhibited considerable expansion in vivo.

There are two primary types of adoptive immunotherapies. Autologous immunotherapy involves isolation, production, and/or expansion of cells such as T cells, (e.g., antigen-specific T cells) from the patient and storage of the patient-harvested cells for re-administration into that same patient as needed. Allogeneic immunotherapy involves two individuals: the patient and a healthy donor. Cells, such as T cells (e.g., antigen-specific T cells), are isolated from the healthy donor and then produced, and/or expanded and banked for administration to a patient with a matching (or partially matching) human leukocyte antigen (HLA) type based on a number of HLA alleles. HLA is also called the Human major histocompatibility complex (MHC). HLA molecules play a key role in transplant immunology where they are critical in matching for organ transplantation, as well as in the adaptive immune response to viruses. HLA class I molecules present viral peptides to CD8+ T cells, and HLA class II molecules present viral peptides to CD4+ T cells.

Allo-HSCT is curative for a variety of malignant and non-malignant hematologic diseases but results in a period of T cell immunodeficiency that leaves patients vulnerable to an array of viruses including cytomegalovirus, adenovirus, Epstein-Barr virus, human herpes virus 6, and BK virus. Several studies have confirmed the antiviral activity of adoptively transferred allogeneic T cells mediated through shared HLA alleles, highlighting the critical role of the HLA interaction in the antiviral response of T cells. Allogeneic stem cell transplant donors may be related [usually a closely HLA-matched sibling or half HLA-matched haploidentical donor (e.g. parent donor for their child)] or unrelated (donor who is not related and found to have very close degree of HLA matching). Often, even when patients have a high degree of HLA match with the donor, the recipient requires immunosuppressive medications to mitigate graft-versus-host disease (GVHD).

Graft-versus-host disease (GVHD) is an inflammatory disease that is unique to allogeneic transplantation. It is an attack by transplanted or reconstituting leukocytes against the recipient's tissues. This can occur even if the donor and recipient are HLA-identical because the immune system can still recognize other differences between cells/tissues. Acute GVHD typically occurs in the first 3 months after transplantation and may involve the skin, intestine, or the liver. Corticosteroids such as prednisone are a standard treatment.

Chronic GVHD may also develop after allogeneic transplant and is the major source of late complications. In addition to inflammation, chronic GVHD may lead to the development of fibrosis, or scar tissue, similar to scleroderma, or other autoimmune diseases and may cause functional disability and the need for prolonged immunosuppressive therapy. GVHD is usually mediated by T cells when they react to foreign peptides presented on the MHC of the host. Thus, the use of adoptive T-cell therapies is often limited by barriers imposed by MHC disparity. This disclosure provides solutions to these barriers.

The time-consuming process of HLA typing patients in order to determine which is the “best” partially HLA-matched donor product reduces the usefulness and safety of adoptive immunotherapies. Further, for third-party donor products, there is potential for rapid rejection of the adoptively transferred cells most critical to treat or prevent the disease or condition to be treated, even in the context of a high degree of HLA match. Safe cell therapies that can be readily generated and/or administered to patients in need thereof are a long felt need in the field. This disclosure addresses this and other needs.

### SUMMARY OF THE DISCLOSURE

In an aspect, the present disclosure includes methods for developing donor minibanks comprising cell therapy products such as antigen-specific T cell lines. The present disclosure further provides compositions and methods wherein such donor minibanks, or products contained in such donor minibanks, are combined together to produce a universal antigen-specific T cell product (e.g., a universal virus-specific T cell product). The universal antigen-specific T cell products provided herein comprise populations of antigen-specific T cells. The disclosure provides compositions comprising the universal antigen-specific T cell products, methods for making the universal antigen-specific T cell products, and therapeutic methods of use of the universal antigen-specific T cell products.

In aspects, the present disclosure provides populations of antigen-specific T cells comprising a plurality of antigen-specific T cell lines derived from a plurality of different donors, wherein the HLA type of each donor differs from at least one of the other donors on at least one HLA allele. In embodiments, the HLA type of each donor differs from at least one of the other donors on at least 2 HLA alleles. In embodiments, the HLA type of each donor differs from at least one of the other donors on at least 3 HLA alleles.

In embodiments, the HLA type of each donor differs from at least one of the other donors on at least one class I HLA. In embodiments, the HLA type of each donor differs from at least one other donor on two or more class I HLA alleles. In embodiments, the HLA type of each donor differs from at least one other donor on at least one HLA-A and at least one HLA-B allele. In embodiments, the HLA type of each donor differs from at least one other donor on one or more Class II HLA alleles. In embodiments, the HLA type of each donor differs from at least one other donor on two or more Class II HLA alleles. In embodiments, the HLA type of each donor differs from at least one other donor on one or more of HLA-DPA1, HLA-DPB1, HLA-DQA1, HLA-DQB1, HLA-DRA, and HLA-DRB1. In embodiments, the HLA type of each donor differs from at least one other donor on two or more alleles independently selected from the group consisting of HLA-DPA1, HLA-DPB1, HLA-DQA1, HLA-DQB1, HLA-DRA, and HLA-DRB1. In embodiments, the HLA type of each donor differs from at least one other donor on at least one HLA-DRB1 allele and at least one HLA-DQB1 allele. In embodiments, the plurality of donors have at least 2 different HLA-A alleles, at least 2 different HLA-B alleles, at least 2 different DRB1 alleles, and/or at least 2 different DQB1 alleles.

In embodiments, the plurality of antigen-specific T cell lines are derived from 3 or more different donors, from 4 or more different donors, or from 5 or more different donors. In embodiments, the plurality of antigen-specific T cell lines are derived from 15 or fewer donors, 14 or fewer donors, 13 or fewer donors, 12 or fewer donors, 11 or fewer donors, 10 or fewer donors, 9 or fewer donors, 8 or fewer donors, 7 or fewer donors, 6 or fewer donors, or 5 or fewer donors (e.g., between 2 and 5 donors). In embodiments, the plurality of antigen-specific T cell lines are derived from 2, 3, 4, 5, 6, 7, 8, 9, 10, 11, 12, 13, 14, 15, 16, 17, 18, 19, or 20 different donors.

In embodiments, the HLA type of each donor differs from at least two of the other donors on at least one HLA allele. In embodiments, the HLA type of each donor differs from at least two of the other donors on at least 2 HLA alleles. In embodiments, the HLA type of each donor differs from at least two of the other donors on at least 3 HLA alleles. In embodiments, the HLA type of each donor differs from at least three of the other donors on at least 3 HLA alleles. In embodiments, the HLA type of each donor differs from each other donor on at least one HLA allele. In embodiments, the donors in the plurality of donors are unrelated to one another. In embodiments, one or more donors in the plurality of donors are related to one another. In embodiments, the plurality of donors comprises both related and unrelated donors.

In embodiments, at least one of the plurality of different donors match on at least two HLA alleles with the greatest possible number of patients in a prospective patient population. In embodiments, at least one of the plurality of different donors match on at least three HLA alleles with the greatest possible number of patients in a prospective patient population. In embodiments, the population of antigen-specific T cells comprising a plurality of antigen-specific T cell lines comprises T cells that match on each HLA allele with one or more patients in a prospective patient population.

In aspects, the populations of antigen-specific T cells comprise antigen-specific T cell lines that are clonal, oligoclonal, and/or polyclonal. In embodiments, the populations of antigen-specific T cells comprise antigen-specific T cell lines, wherein one or more of the T cell lines is polyclonal. In embodiments, all of the antigen-specific T cell lines in the population are polyclonal.

In embodiments, the antigen-specific T cell lines from each donor are pooled together after each cell line is generated. In embodiments, the antigen-specific T cell lines are assessed for cell line identity, viability, sterility, phenotype, potency, and/or alloreactivity. In embodiments, the antigen-specific T cell lines are individually assessed for cell line identity, viability, sterility, phenotype, potency, and/or alloreactivity prior to pooling. In embodiments, the potency of the cell line is assessed by phenotype, production of effector molecules, and/or cytolytic function. For example, in embodiments, the potency of the cell line is assessed by production of IFNγ, TNFα, IL-2, and/or Granzyme B. In embodiments, the potency of the cell line is assessed by measuring cytolytic activity against target cells, e.g., in a chromium release assay. In embodiments, the potency of the cell line is assessed by determining the phenotype of the cells. For example, in embodiments, the potency of the cell line is assessed by measuring upregulation of activation and/or degranulation markers (e.g., CD25, CD69, CD62L, CD44, CD28, and/or CD107a). In embodiments, the phenotype is determined by flow cytometry. In embodiments, each cell line comprises at least 75%, at least 80%, at least 85%, at least 90%, or at least 95% CD3+ T cells. In embodiments, each cell line comprises at least 90% CD3+ T cells. In embodiments, the sterility of each cell line is determined by testing for bacterial contamination, fungal contamination, mycoplasma, and/or endotoxin levels. In further embodiments, each cell line has an endotoxin level of less than 5 EU/mL. In embodiments, the alloreactivity of each antigen-specific T cell line against unrelated and/or partially HLA matched and/or HLA unmatched target cells is assessed by chromium release assay. In embodiments, after the antigen-specific T cell lines are pooled, the pool of cell lines is HLA typed. In embodiments, the pool of cell lines is tested for functional responses using HLA-restricted epitopes.

In embodiments, the antigen-specific T cell lines are pooled together at a ratio of about 1:1. In embodiments, where the population of antigen-specific cells comprises two different antigen-specific T cell lines, the T cell lines are pooled together at a ratio ranging from about 10:1 to about 1:10 for each cell line relative to another cell line, e.g. about 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, or 1:10. In embodiments, where the population of antigen-specific T cells comprise more than two different T cell lines, any ratios within the aforementioned ranges may be utilized. For example, in embodiments, a population of antigen-specific T cells may comprise four different T cell lines pooled together at a ratio of about 4:3:2:1. In embodiments, the antigen-specific T cell lines are pooled together at a ratio of about 1:1 for each T cell line in the population, e.g., for a population comprising four different T cell lines, a ratio of about 1:1:1:1.

In embodiments, the antigen-specific T cell lines from each donor are pooled together as fresh cell lines without any freeze-thaw or cryopreservation step. In further embodiments, the pooled product is cryopreserved. In embodiments, the antigen-specific T cell lines from each donor are pooled together after each cell line has been individually cryopreserved and then subsequently thawed. In embodiments, the antigen-specific T cell lines from each donor have been tested for cell line identity, viability, sterility, phenotype, potency, and/or alloreactivity; then cryopreserved as individual cell lines; subsequently thawed; and then pooled together to generate a universal antigen-specific T cell therapy product. Optionally, the resulting universal antigen-specific T cell therapy product may be utilized as a cell therapy without a further freeze-thaw step, or may be cryopreserved for later use as a cell therapy product.

In embodiments, the population of antigen-specific T cells comprises from about 10×10⁶ to about 100×10⁶ T cells. In embodiments, the population comprises about 10×10⁶, about 20×10⁶, about 30×10⁶, about 40×10⁶, about 50×10⁶, about 60×10⁶, about 70×10⁶, about 80×10⁶, about 90×10⁶, or about 100×10⁶ T cells. In embodiments, the population comprises about 45×10⁶ T cells. In embodiments, the present disclosure provides a composition comprising a population of antigen-specific T cells comprising about 2.5×10⁶ T cells/mL to about 25×10⁶ T cells/mL.

In embodiments, the population of antigen-specific T cells comprises T cells that are specific for one or more viral antigens or one or more tumor associated antigens. In embodiments, the antigen-specific T cells are virus-specific T cells (VSTs). In embodiments, the present disclosure provides a universal VST product, referred to herein as UVSTs. In embodiments, the one or more viral antigens are from one or more viruses selected from the group consisting of Epstein Barr virus (EBV), cytomegalovirus (CMV), Adenovirus (AdV), BK virus (BKV), JC virus, human herpesvirus 6 (HHV6), respiratory syncytial virus (RSV), influenza, parainfluenza, bocavirus, coronavirus, lymphocytic choriomeningitis virus (LCMV), mumps, measles, human metapneumovirus (hMPV), parvovirus B, rotavirus, merkel cell virus, herpes simplex virus (HSV), hepatitis B virus (HBV), hepatitis C virus (HCV), hepatitis D virus (HDV), human papilloma virus (HPV), human immunodeficiency virus (HIV), human T-cell leukemia virus type 1 (HTLV1), human herpesvirus 8 (HHV8), West Nile virus, zika virus, and ebola virus. In embodiments, the one or more viral antigens comprise antigens from BKV, CMV, AdV, EBV, and HHV-6. In embodiments, the one or more viral antigens comprise antigens from RSV, influenza, parainfluenza, and hMPV. In embodiments, the one or more viral antigens comprise antigens from a coronavirus. In embodiments, the coronavirus is SARS-Cov-2. In embodiments, the one or more viral antigens comprise antigens from HBV. In embodiments, the one or more viral antigens comprise antigens from HHV-8.

In embodiments, the one or more tumor associated antigens are selected from the group consisting of CEA, MHC, CTLA-4, gp100, mesothelin, PD-L1, TRP1, CD40, EGFP, Her2, TCR alpha, trp2, TCR, MUC1, cdr2, ras, 4-1BB, CT26, GITR, OX40, TGF-α. WT1, MUC1, LMP2, HPV E6 E7, EGFRvIII, HER-2/neu, MAGE A3, p53 nonmutant, NY-ESO-1, PSMA, GD2, Melan A/MART1, Ras mutant, gp 100, p53 mutant, Proteinase3 (PR1), bcr-abl, Tyrosinase, Survivin, PSA, hTERT, EphA2, PAP, ML-IAP, AFP, EpCAM, ERG (TMPRSS2 ETS fusion gene), NA17, PAX3, ALK, Androgen receptor, Cyclin B1, Polysialic acid, MYCN, RhoC, TRP-2, GD3, Fucosyl GM1, Mesothelin, PSCA, MAGE A1, sLe(a), CYP1B1, PLAC1, GM3, BORIS, Tn, GloboH, ETV6-AML, NY-BR-1, RGS5, SART3, STn, Carbonic anhydrase IX, PAX5, OY-TES1, Sperm protein 17, LCK, HMWMAA, AKAP-4, SSX2, XAGE 1, B7H3, Legumain, Tie 2, Page4, VEGFR2, MAD-CT-1, FAP, PDGFR-β, MAD-CT-2, and Fos-related antigen1.

The present disclosure provides compositions comprising a population of antigen-specific T cells provided herein. In embodiments, the present disclosure provides a universal antigen specific T cell composition comprising a population of antigen-specific T cells provided herein and/or comprising antigen-specific T cell lines provided herein. For example, in embodiments the present disclosure provides a universal VST (UVST). In embodiments, the compositions provided are or have been cryopreserved. In embodiments, the compositions comprise a cryopreservation media. In embodiments, the cryopreservation media comprises human serum albumin, Hank's balanced salt solution (HBSS), and dimethyl sulfoxide (DMSO). In embodiments, the media comprises about 10% (v/v) DMSO. In embodiments, the cryopreservation media comprises about 50% (v/v) of 25% human serum albumin and about 40% (v/v) HBSS.

In embodiments, the population of antigen-specific T cells has been modified to express an exogenous molecule. For example, in embodiments, the exogenous molecule is a therapeutic agent. In further embodiments, the therapeutic agent is a chemotherapeutic drug, cytokine, chemokine, small molecule inhibitor of tumor growth, or a molecule that sequesters immune inhibitor molecules. In embodiments, the exogenous molecule is a transgenic molecule. Thus, in embodiments, the present disclosure provides a population of antigen-specific T cells, wherein antigen-specific T cells within the population have been transduced with a transgene encoding an exogenous molecule. In embodiments, the transgenic molecule comprises an extracellular binding domain, a transmembrane domain, and a signaling domain. In embodiments, the extracellular binding domain is specific for a cancer antigen. In embodiments, the transgenic molecule is a chimeric antigen receptor (CAR), a T cell receptor (TCR), or an NK cell receptor (e.g., NKG2D). In embodiments, T cells within one or more of the antigen-specific T cell lines have been modified to express the exogenous molecule. In embodiments, T cells within all of the antigen-specific T cell lines in the population have been modified to express the exogenous molecule. In embodiments, T cells within the pooled population have been modified to express the exogenous molecule. In embodiments, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 85%, or at least about 90% of the T cells in the T cell line and/or in the population of T cell lines express the exogenous molecule.

In embodiments, the present disclosure provides a universal antigen specific T cell therapy product comprising the population of antigen-specific T cells provided herein. In embodiments, the product exhibits a lack of alloreactivity to partially HLA-matched and/or to HLA mismatched target cells. In embodiments, the product exhibits a lack of alloreactivity against cells in a target population. In embodiments, the product may be in the form of a composition comprising the population of antigen-specific T cells making up the product. In embodiments, the product may be in the form of separate compositions each comprising one or more antigen-specific T cell line. In such embodiments, the separate compositions are for administration to a patient in a single dosing session as further described herein.

In embodiments, the universal antigen-specific T cell therapy product comprises antigen-specific T cell lines of sufficient HLA diversity with respect to one another that they collectively provide at least one antigen specific T cell line that is matched on at least 2 HLA alleles with at least about 70%, at least about 75%, at least about 80%, at least about 85%, at least about 90%, at least about 95%, or at least about 99% of a prospective patient population.

In aspects, the present disclosure provides methods for treating a disease or condition in a patient, comprising administering to the patient a population of antigen-specific T cell lines, a composition, or a universal antigen specific T cell therapy product provided herein. In embodiments, the population, composition, or T cell therapy product comprises a mixture of T cells, wherein the mixture of T cells comprises T cells that are partially HLA matched, T cells that are partially HLA mismatched, and T cells that are completely mismatched with the HLA type of the patient.

In aspects, the present disclosure provides methods for treating a disease or condition in a patient, comprising administering to the patient universal antigen specific T cell therapy in a single dosing session. In embodiments, the universal antigen-specific T cell therapy is in the form of one composition comprising the population of antigen-specific T cells. In embodiments, the universal antigen-specific T cell therapy is in the form of separate compositions, each composition comprising one or more individual T cell lines. In such embodiments, the methods comprise administering to the patient a plurality of antigen-specific T cell lines from a plurality of different donors, wherein the HLA type of each donor differs from at least one of the other donors on at least on HLA allele, and wherein the method comprises administering the plurality of antigen-specific T cell lines to the patient in a single dosing session.

In embodiments, administering in a single dosing session comprises administering the plurality of antigen-specific T cell lines to the patient simultaneously in the same composition. In embodiments, administering in a single dosing session comprises administering the plurality of antigen-specific T cell lines to the patient in separate compositions administered sequentially. In embodiments, sequential administrations are performed within 5 minutes of one another, or within 30 minutes of one another, or within 1 hour of one another. In embodiments, sequential administrations are administered in a single dosing session such that the patient receives all administrations on the same day and does not undergo testing for the efficacy and/or longevity of one or more T cell lines prior to administration of one or more additional T cell lines of the universal antigen-specific T cell therapy.

In embodiments, the methods provided herein comprise administering a universal antigen specific T cell therapy to a subject, wherein the universal antigen-specific T cell therapy comprises a mixture of T cells comprising T cells that are partially matched with the HLA type of the patient and T cells that are completely mismatched with the HLA type of the patient.

In embodiments, the methods provided herein comprise administering to the patient a dose of about 10×10⁶ to about 100×10⁶ antigen-specific T cells, or about 20×10⁶ to about 80×10⁶ antigen-specific T cells, or about 30×10⁶ to about 60×10⁶ antigen-specific T cells, or about 40×10⁶ to about 50×10⁶ antigen-specific T cells. In embodiments, the methods comprise administering to the patient a dose of about 45×10⁶ T cells.

In embodiments, the methods comprise pooling together or otherwise administering to a patient in a single dosing session two or more individual antigen-specific T cell products, or pooling together or otherwise administering in a single dosing session one or more universal antigen-specific T cell products with one or more individual antigen-specific T cell lines.

In embodiments, the disease or condition is a viral infection. In embodiments, the antigen-specific T cells are virus-specific T cells (VSTs). In embodiments, the method achieves a reduction in viral load in the patient and/or reduction or elimination of symptoms of a disease associated with the viral infection. In embodiments, the method achieves a faster resolution of viral infection relative to a patient that did not receive the VSTs.

In embodiments, the patient is immunocompromised. In embodiments, the patient is immunocompromised due to a treatment the patient received to treat the disease or condition or another disease or condition. In embodiments, the patient is immunocompromised due to age. In embodiments, patent is immunocompromised due to young age or old age.

In embodiments, the condition is an immune deficiency. In embodiments, the immune deficiency is primary immune deficiency. In embodiments, the patient is in need of a transplant. In embodiments, the patient has received a transplant.

In embodiments, the disease or condition is a cancer. In embodiments, the cancer is selected from the group consisting of lung cancer, bowel cancer, colon cancer, rectal cancer, bile duct cancer, pancreatic cancer, testicular cancer, prostate cancer, ovarian cancer, breast cancer, melanoma, soft tissue sarcoma, lymphoma, leukemia, and multiple myeloma.

In aspects, the present disclosure provides methods for generating a universal antigen specific T cell therapy product comprising a population of antigen-specific T cells, the method comprising (i) culturing mononuclear cells from each donor of a plurality of donors (e.g., a plurality donors wherein the HLA type of each donor differs from at least one of the other donors on at least one HLA allele, and/or a plurality of suitable donors for inclusion in a donor minibank as described herein), each in a separate culture in the presence of one or more cytokines and one or more antigen, to generate a plurality of individual cell lines of expanded antigen-specific T cells, and (ii) pooling together the individual cell lines to generate the universal antigen specific T cell therapy product. In embodiments, the mononuclear cells are peripheral blood mononuclear cells (PBMC). In embodiments, the methods further comprise one or more freeze-thaw step. For example, in embodiments, each cell line is cryopreserved and then thawed prior to the pooling of (ii). In other embodiments, each cell line is pooled together as a freshly prepared cell line, without any freeze-thaw step prior to the pooling of (ii) In embodiments, the methods comprise freezing the pool of cell lines obtained in (ii). In embodiments, the methods comprise generating the plurality of individual cell lines as provided in (i), determining the cell line identity, viability, sterility, phenotype, potency, and/or alloreactivity, freezing the individual cell lines, subsequently thawing the individual cell lines, and pooling together the cell lines to form a universal antigen-specific T cell therapy product. Alternatively, the methods comprise generating the plurality of individual cell lines as provided in (i), freezing the individual cell lines, thawing the individual cell lines and then determining the cell line identity, viability, sterility, phenotype, potency, and/or alloreactivity, then either pooling individual cell lines together to form the universal antigen-specific T cell therapy product, or re-freezing the individual cell lines prior to subsequent thaw and pooling together to form the universal antigen-specific T cell therapy product. In embodiments, the disclosure provides a method for generating a universal antigen specific T cell therapy product comprising a population of antigen-specific T cells, the method comprising (i) pooling mononuclear cells from each donor of a plurality of donors (e.g., a plurality donors wherein the HLA type of each donor differs from at least one of the other donors on at least one HLA allele, and/or a plurality of suitable donors for inclusion in a donor minibank as described herein), and (ii) culturing the pool of mononuclear cells in the presence of one or more cytokines and one or more antigen, to generate a population of expanded antigen-specific T cells. In embodiments, the mononuclear cells are peripheral blood mononuclear cells (PBMC). In embodiments, the cell line identity, viability, sterility, phenotype, potency, and/or alloreactivity of the pooled cells is determined as provided herein. In embodiments, the pooled cells may be frozen after step (i) and/or (ii).

Thus, in embodiments, the methods provided herein comprise freezing individual antigen-specific T cell lines and/or pooled universal antigen-specific T cell therapy products in cryopreservation medium. In embodiments, the cryopreservation medium comprises human serum albumin, Hank's balanced salt solution (HBSS), and dimethyl sulfoxide (DMSO). In embodiments, the medium comprises about 10% (v/v) DMSO. In embodiments, the medium comprises about 50% (v/v) of 25% human serum albumin and about 40% (v/v) HBSS. In embodiments, the pooled universal antigen-specific T cell therapy product is cryopreserved and stored until it is selected for use in a method to treat a disease or condition in a patient. Accordingly, the present disclosure provides compositions comprising antigen-specific T cell lines and/or pooled universal antigen-specific T cell therapy products in cryopreservation medium. In embodiments, the methods further comprise one or more filtration step. In embodiments, the methods further comprise filtering each cell line obtained in step (i) in the preceding paragraph. In embodiments, the methods further comprise filtering the pooled universal antigen specific T cell therapy product obtained in (ii) in the preceding paragraph. In embodiments, the methods further comprise filtering each cell line and/or filtering the pooled universal antigen specific T cell therapy product, before and/or after a freeze-thaw step.

In embodiments, the methods further comprise transfecting one or more individual cell line obtained in (i) of the two preceding paragraphs with a transgene. In embodiments, the methods further comprise transfecting the pooled cell lines obtained in (ii) of the two preceding paragraphs with a transgene. In embodiments, the transgene encodes a chimeric antigen receptor (CAR), a T cell receptor (TCR), or an NK cell receptor.

In embodiments, the culturing step of the methods of production provided herein are performed in a vessel comprising a gas permeable culture surface. In embodiments, the vessel is a GRex bioreactor. In embodiments, the one or more cytokines cultured with the mononuclear cells and antigens is selected from the group consisting of IL-1, IL-2, IL-4, IL-6, IL-7, IL-12, IL-15, IL-21, and a combination thereof. In embodiments, the one or more cytokines cultured with the mononuclear cells and antigens is selected from the group consisting of IL-1, IL-4, IL-6, IL-7, IL-12, IL-15, IL-21, and a combination thereof, and wherein the cytokines do not comprise IL-2. In embodiments, the one or more cytokines cultured with the mononuclear cells and antigens is IL-4 and/or IL-7. In embodiments, the cytokines comprise IL-4 and IL-7 and do not comprise IL-2.

In embodiments, the one or more antigen is in the form of (a) a whole protein, (b) a pepmix comprising a series of overlapping peptides spanning part of or the entire sequence of each antigen, or (c) a combination of (a) and (b). In embodiments, the antigens comprise at least 2, 3, 4, 5, 6, 7, 8, 9, 10, 11, 12, 13, 14, 15, 16, 17, 18, 19, 20, or more different pepmixes.

In embodiments, the one or more antigens are viral antigens or tumor associated antigens. In embodiments, each antigen in the culture is a viral antigen. In embodiments, the viral antigens are from a virus selected from EBV, CMV, Adenovirus, BK, JC virus, HHV6, RSV, influenza, parainfluenza, bocavirus, coronavirus, LCMV, mumps, measles, human metapneumovirus, parvovirus B, rotavirus, merkel cell virus, HSV, HBV, HCV, HDV, HPV, HIV, HTLV1, HHV8, West Nile virus, zika virus, and ebola virus.

In embodiments, each antigen in the culture is a tumor associated antigen. In embodiments, the tumor associated antigens are one or more of CEA, MHC, CTLA-4, gp100, mesothelin, PD-L1, TRP1, CD40, EGFP, Her2, TCR alpha, trp2, TCR, MUC1, cdr2, ras, 4-1BB, CT26, GITR, OX40, TGF-α. WT1, MUC1, LMP2, HPV E6 E7, EGFRvIII, HER-2/neu, MAGE A3, p53 nonmutant, NY-ESO-1, PSMA, GD2, Melan A/MART1, Ras mutant, gp 100, p53 mutant, Proteinase3 (PR1), bcr-abl, Tyrosinase, Survivin, PSA, hTERT, EphA2, PAP, ML-IAP, AFP, EpCAM, ERG (TMPRSS2 ETS fusion gene), NA17, PAX3, ALK, Androgen receptor, Cyclin B1, Polysialic acid, MYCN, RhoC, TRP-2, GD3, Fucosyl GM1, Mesothelin, PSCA, MAGE A1, sLe(a), CYP1B1, PLAC1, GM3, BORIS, Tn, GloboH, ETV6-AML, NY-BR-1, RGS5, SART3, STn, Carbonic anhydrase IX, PAX5, OY-TES1, Sperm protein 17, LCK, HMWMAA, AKAP-4, SSX2, XAGE 1, B7H3, Legumain, Tie 2, Page4, VEGFR2, MAD-CT-1, FAP, PDGFR-β, MAD-CT-2, and Fos-related antigen1.

In an aspect, the present disclosure includes methods for developing donor minibanks comprising cell therapy products such as antigen-specific T cell lines and the universal antigen-specific T cell products provided herein. In embodiments, the present disclosure includes methods for identifying one or more suitable donors from at least one donor pool that have various HLA (Human Leukocyte Antigen) allele types compatible with the majority of prospective patients. In some embodiments, the prospective patients have undergone allogeneic hematopoietic stem cell transplantation (HSCT). In some embodiments, the prospective patients have suppressed immunity or are immunocompromised. In various embodiments, methods in the present disclosure concern the restoration of T cell immunity of patients who are immunocompromised.

In some embodiments, the identification of one or more suitable donors in methods of the disclosure concern the construction of a first donor minibank containing a plurality of cell therapy products. In some embodiments, the first donor minibank contains antigen-specific T cell lines. In some embodiments, methods in the present disclosure include a donor selection method. In some embodiments, the donor selection method comprises (a) comparing an HLA type of each of a first plurality of potential donors from a first donor pool with each of a first plurality of prospective patients from a first prospective patient population; (b) determining, based on the comparison in the above-mentioned step (a), a first greatest matched donor, wherein the first greatest matched donor can be defined as the donor from the first donor pool that has 2 or more HLA allele matches with the greatest number of patients in the first plurality of prospective patients; (c) selecting the first greatest matched donor for inclusion in the first donor minibank; (d), removing from the first donor pool the first greatest matched donor; wherein the above-mentioned step (d) can generate a second donor pool consisting of each of the first plurality of potential donors from the first donor pool except for the first greatest matched donor; (e) removing from the first plurality of prospective patients each prospective patient that has 2 or more allele matches with the first greatest matched donor, wherein the above-mentioned step (e) comprises generating a second plurality of prospective patients consisting of each of the first plurality of prospective patients except for each prospective patient that has 2 or more allele matches with the first greatest matched donor; and (f) repeating the foregoing steps (a) through (e) one or more additional times with all donors and prospective patients that have not already been removed in accordance with the foregoing steps (d) and (e). In some embodiments, each time an additional greatest matched donor is selected in accordance with the foregoing step (c) that additional greatest matched donor is removed from their respective donor pool in accordance with the foregoing step (d). In some embodiments, each time a subsequent greatest matched donor is removed from their respective donor pool, each prospective patient that has 2 or more allele matches with that subsequent greatest matched donor is removed from their respective plurality of prospective patients in accordance with the foregoing step (e). In some embodiments, methods as described herein can sequentially increase the number of selected greatest matched donors in the first donor minibank by 1 following each cycle of the method. In some embodiments, methods as described herein can deplete the number of the plurality of prospective patients in the patient population following each cycle of the method in accordance with their HLA matching to the selected greatest matched donors. In other embodiments, the foregoing steps (a) through (e) can be repeated until a desired percentage of the first prospective patient population remains in the plurality of prospective patients. In other embodiments, the foregoing steps (a) through (e) can be repeated until no donors remain in the donor pool.

The present disclosure provides that the foregoing steps (a)-(e) of methods as described herein can be cycled in accordance with the foregoing step (f) until 5% or less of the first prospective patient population remains in the plurality of prospective patients. In some embodiments, the first donor minibank as described herein can comprise antigen-specific T cell lines derived from 10 or less donors. In some embodiments, the first donor minibank as described herein can comprise antigen-specific T cell lines derived from 10, 9, 8, 7, 6, 5, 4, 3, or 2 donors. In some embodiments, the first donor minibank as described herein can comprise enough HLA variability to provide >95% of the first prospective patient population with one or more antigen-specific T cell line that is matched to the patient's HLA type on at least 2 HLA alleles. In other embodiments, the first donor minibank as described herein can comprise antigen-specific T cell lines derived from 5 or less donors. In some embodiments, the first donor minibank as described herein can provide enough HLA variability to provide >95% of the first prospective patient population with one or more antigen-specific T cell line that is matched to the patient's HLA type on at least 2 HLA alleles. In some embodiments, the 2 or more alleles from the foregoing steps (b) and (e) can comprise at least 2 HLA Class I alleles. In some embodiments, the 2 or more alleles from the foregoing steps (b) and (e) can comprise at least 2 HLA Class II alleles. In some embodiments, the 2 or more alleles from the foregoing steps (b) and (e) can comprise at least 1 HLA Class I allele and at least 1 HLA Class II allele. In some embodiments, the 2 or more alleles from the foregoing steps (b) and (e) can comprise the HLA alleles HLA A, HLA B, DRB1, and DQB1.

In some embodiments, the first donor pool used in the present disclosure can comprise at least 10 donors. In some embodiments, the first prospective patient population provided in the present disclosure can comprise at least 100 patients. In some embodiments, the first prospective patient population can comprise the entire worldwide allogeneic HSCT population. In some embodiments, the first prospective patient population can comprise the entire US allogeneic HSCT population. In some embodiments, the first prospective patient population can comprise all patients included in the National Marrow Donor Program (NMDP) database, available at the worldwide web address bioinformatics.bethematchclinical.org. In some embodiments, the first prospective patient population can comprise all patients included in the European Society for Blood and Marrow Transplantation (EBMT) database, available at the worldwide web address: ebmt.org/ebmt-patient-registry. In some embodiments, the entire worldwide allogeneic HSCT population can include children ages ≤16 years. In some embodiments, the entire US allogeneic HSCT population can include children ages ≤16 years. In some embodiments, the entire worldwide allogeneic HSCT population can include individuals ages ≥65. In some embodiments, the entire US allogeneic HSCT population can include individuals ages ≥65. In some embodiments, the entire worldwide allogeneic HSCT population can include children ages ≤5 years. In some embodiments, the entire US allogeneic HSCT population can include children ages ≤5 years.

The present disclosure provides methods of identifying suitable donors for use in constructing a donor bank made up of a plurality of minibanks of antigen-specific T cell lines. In some embodiments, constructing a donor bank can comprise first developing a first minibank as described herein. In some embodiments, developing a first minibank can include performing all of the foregoing steps (a)-(f). In some embodiments, developing a first minibank for a donor bank as described herein can comprise repeating the foregoing steps (a) through (f) that involves one or more second rounds to construct one or more second minibanks.

In some embodiments, prior to starting each second round for constructing a bank can comprise generating a new donor pool. In some embodiments, the new donor pool as described herein can comprise the first donor pool, less any greatest matched donors removed in accordance with each prior cycle of the forgoing step (d) from the first and any prior second rounds. In some embodiments, the new donor pool as described herein can comprise an entirely new population of potential donors not included in the first donor pool. In some embodiments, the new donor pool as described herein can comprise a combination of the first donor pool, less any greatest matched donors removed in accordance with each prior cycle of the forgoing step (d) from the first and any prior second rounds and an entirely new population of potential donors not included in the first donor pool. In some embodiments, constructing a bank as described in the present method can comprise reconstituting the first plurality of prospective patients from the first prospective patient population by returning all prospective patients that had been previously removed in accordance with each prior cycle of the foregoing step (e) from the first and any prior second rounds of the method.

In some embodiments, each round for constructing one or more minibanks as described herein can include cycling the above-identified steps (a) through (e) in accordance with the above-identified step (f) until 5% or less of the first prospective patient population remains in the plurality of prospective patients. In some embodiments, each donor minibank can comprise enough HLA variability amongst the one or more greatest matched donors to provide >95% of the first prospective patient population with at least one antigen-specific T cell line that is matched to the patient's HLA type on at least 2 HLA alleles. In some embodiments, each resulting donor minibank can comprise antigen-specific T cell lines derived from 10 or less donors. In some embodiments, each resulting donor minibank can comprise antigen-specific T cell lines derived from 5 or less donors. In some embodiments, the 2 or more alleles from the foregoing steps (b) and (e) can comprise at least 2 HLA Class II alleles. In other embodiments, the 2 or more alleles from the foregoing steps (b) and (e) can comprise at least 1 HLA Class I allele and at least 1 HLA Class II allele.

In some embodiments, the first donor pool used for constructing a donor bank can comprise at least 10 donors. In some embodiments, the first prospective patient population used for constructing a donor bank can comprise at least 100 patients. In some embodiments, the first prospective patient population can comprise the entire worldwide allogeneic HSCT population In some embodiments, the first prospective patient population can comprise the entire US allogeneic HSCT population. In some embodiments, the first prospective patient population can comprise all patients included in the National Marrow Donor Program (NMDP) database, available at the worldwide web address bioinformatics.bethematchclinical.org. In some embodiments, the first prospective patient population can comprise all patients included in the European Society for Blood and Marrow Transplantation (EBMT) database, available at the worldwide web address: ebmt.org/ebmt-patient-registry. In some embodiments, the entire worldwide allogeneic HSCT population can include children ages ≤16 years. In some embodiments, the entire US allogeneic HSCT population can include children ages ≤16 years. In some embodiments, the entire worldwide allogeneic HSCT population can include individuals ages ≥65. In some embodiments, the entire US allogeneic HSCT population can include individuals ages ≥65. In some embodiments, the entire worldwide allogeneic HSCT population can include children ages ≤5 years. In some embodiments, the entire US allogeneic HSCT population can include children ages ≤5 years.

In some embodiments, methods as described herein can comprise harvesting blood from each donor included in the donor bank. In other embodiments, methods as described herein can comprise having blood harvested from each donor included in the donor bank. In some embodiments, methods as described herein can comprise harvesting mononuclear cells (MNCs) from each donor included in the donor bank. In some embodiments, methods as described herein can comprise having MNCs harvested from each donor included in the donor bank. In some embodiments, harvesting MNCs from each donor can comprise isolating the MNCs or having the MNCs isolated. In one embodiment, the MNCs comprise peripheral blood mononuclear cells (e.g., PBMCs). In one embodiment, the MNCs comprise blood apheresis mononuclear cells. In some embodiments, harvesting MNCs from each donor can comprise isolating the PBMCs or having the PBMCs isolated. In some embodiments, isolating MNCs can be conducted by ficoll gradient. In some embodiments, isolating MNCs can be conducted by density gradient. In other embodiments, harvesting MNCs as disclosed herein can comprise culturing the cells. In other embodiments, harvesting MNCs as disclosed herein can comprise cryopreserving the cells.

In some embodiments, the cultured MNCs or the cryopreserved MNCs can comprise contacting the cells in culture with one or more antigens under suitable culture conditions to stimulate and expand antigen-specific T cells. In other embodiments, the one or more antigen contacted with the cells can comprise one or more viral antigens. In other embodiments, the one or more antigen contacted with the cells can comprise one or more tumor associated antigens. In some embodiments, the one or more antigen contacted with the cells can comprise a combination of one or more viral antigen and one or more tumor associated antigen.

The present disclosure provides methods of constructing a first donor minibank of antigen-specific T cell lines. In some embodiments, the methods can include step (a) of comparing the HLA type of each of the first plurality of potential donors with each of the first plurality of prospective patients. In some embodiments, the methods can include step (b) of determining, based on the comparison in step (a) of the methods described in this paragraph, a first greatest matched donor. In some embodiments, first greatest matched donor can be defined as the donor from the first donor pool that has 2 or more allele matches with the greatest number of patients in the first plurality of prospective patients. In some embodiments, the methods can comprise step (c) of selecting the first greatest matched donor for inclusion in the first donor minibank. In some embodiments, the methods can comprise step (d) of removing from the first donor pool the first greatest matched donor. In some embodiments, step (d) of the methods as described herein can comprise generating a second donor pool consisting of each of the first plurality of potential donors from the first donor pool except for the first greatest matched donor.

In some embodiments, the methods can comprise step (e) of removing from the first plurality of prospective patients each prospective patient that has 2 or more allele matches with the first greatest matched donor. In some embodiments, step (e) as described in this paragraph can generate a second plurality of prospective patients consisting of each of the first plurality of prospective patients except for each prospective patient that has 2 or more allele matches with the first greatest matched donor.

In some embodiments, the methods of constructing a first donor minibank of antigen-specific T cell lines can comprise repeating steps (a) through (e) as disclosed herein one or more additional times with all donors and prospective patients that have not already been removed in accordance with steps (d) and (e) as disclosed herein. In some embodiments, each time an additional greatest matched donor is selected in accordance with step (c) that greatest matched donor is removed from their respective donor pool in accordance with step (d). In some embodiments, each time a subsequent greatest matched donor is removed from their respective donor pool, each prospective patient that has 2 or more allele matches with that subsequent greatest matched donor is removed from their respective plurality of prospective patients in accordance with step (e). In some embodiments, the methods as described herein can sequentially increase the number of selected greatest matched donors in the donor minibank by 1 following each cycle of the method. In some embodiments, the methods as described herein can deplete the number of the plurality of prospective patients in the patient population following each cycle of the method in accordance with their HLA matching to the selected greatest matched donors. In some embodiments, steps (a) through (e) for constructing a first donor minibank of antigen-specific T cell lines can be repeated until a desired percentage of the first prospective patient population remains in the plurality of prospective patients. In some embodiments, steps (a) through (e) for constructing a first donor minibank of antigen-specific T cell lines can be repeated until no donors remain in the donor pool.

In some embodiments, methods as described herein comprise step (g) isolating MNCs, or having MNCs, isolated, from blood obtained from each respective donor included in the donor minibank. In some embodiments, step (h) of the methods as described herein comprise culturing the MNCs obtained from each respective donor. In some embodiments, methods as described herein comprise step (i) of contacting the MNCs in culture with one or more antigen under suitable culture conditions to stimulate and expand a polyclonal population of antigen-specific T cells from each of the respective donor's MNCs. In some embodiments, methods as described herein comprise step (i) of contacting the MNCs in culture with one or more epitope from one or more antigen, under suitable culture conditions to stimulate and expand a polyclonal population of antigen-specific T cells from each of the respective donor's MNCs. In some embodiments, methods as described herein comprise producing a plurality of antigen-specific T cell lines. In some embodiments, each of antigen-specific T cell lines can comprise a polyclonal population of antigen-specific T cells derived from each respective donor's MNCs. In some embodiments, the MNCs of steps (g) through (i) as described herein can be PBMCs. In some embodiments, step (j) of the methods can comprise cryopreserving the plurality of antigen-specific T cell lines.

In some embodiments, methods of constructing a first donor minibank of antigen-specific T cell lines as described herein can include cycling steps (a) through (e) in accordance with step (f) until 5% or less of the first prospective patient population remains in the plurality of prospective patients. In some embodiments, each donor minibank can comprise enough HLA variability amongst the one or more greatest matched donors to provide >95% of the first prospective patient population with at least one antigen-specific T cell line that is matched to the patient's HLA type on at least 2 HLA alleles. In some embodiments, each resulting donor minibank can comprise antigen-specific T cell lines derived from 10 or less donors. In some embodiments, each resulting donor minibank can comprise antigen-specific T cell lines derived from 5 or less donors. In some embodiments, the 2 or more alleles from steps (b) and (e) can comprise at least 2 HLA Class II alleles. In other embodiments, the 2 or more alleles from steps (b) and (e) can comprise at least 1 HLA Class I allele and at least 1 HLA Class II allele.

In some embodiments, the first donor pool used in the methods of constructing a first donor minibank of antigen-specific T cell lines as described herein can comprise at least 10 donors. In some embodiments, the first donor pool used in the methods of constructing a first donor minibank of antigen-specific T cell lines as described herein can comprise at least 100 donors. In some embodiments, the first prospective patient population can comprise the entire worldwide allogeneic HSCT population. In some embodiments, the first prospective patient population used in the methods can comprise the entire US allogeneic HSCT population. In some embodiments, the first prospective patient population can comprise all patients included in the National Marrow Donor Program (NMDP) database, available at the worldwide web address bioinformatics.bethematchclinical.org. In some embodiments, the first prospective patient population can comprise all patients included in the European Society for Blood and Marrow Transplantation (EBMT) database, available at the worldwide web address: ebmt.org/ebmt-patient-registry. In some embodiments, the entire worldwide allogeneic HSCT population can include children ages ≤16 years. In some embodiments, the entire US allogeneic HSCT population can include children ages ≤16 years. In some embodiments, the entire worldwide allogeneic HSCT population can include individuals ages ≥65. In some embodiments, the entire US allogeneic HSCT population can include individuals ages ≥65. In some embodiments, the entire worldwide allogeneic HSCT population can include children ages ≤5 years. In some embodiments, the entire US allogeneic HSCT population can include children ages ≤5 years.

In some embodiments, the culturing of MNCs can be in a vessel comprising a gas permeable culture surface. In one embodiment, the vessel can be an infusion bag with a gas permeable portion. In one embodiment, the vessel can be a rigid vessel. In one embodiment, the vessel can be a GRex bioreactor. In some embodiments, culturing the PBMCs for constructing a first donor minibank of antigen-specific T cell lines as described herein can be conducted in the presence of one or more cytokine. In one embodiment, the cytokine can include IL4. In one embodiment, the cytokine can include IL7. In one embodiment, the cytokine can include IL4 and IL7. In one embodiment, the cytokine can include IL4 and IL7, but not IL2.

Methods of constructing a first donor minibank of antigen-specific T cell lines can comprise culturing the MNCs in the presence of one or more antigen. In one embodiment, the MNCs can be PBMCs. In some embodiments, the one or more antigen can be in the form of a whole protein. In some embodiments, the one or more antigen can be in the form of a pepmix comprising a series of overlapping peptides spanning part of or the entire sequence of each antigen. In some embodiments, the one or more antigen can be in the form of a combination of the form of a whole protein and the form of a pepmix comprising a series of overlapping peptides spanning part of or the entire sequence of each antigen.

Methods of constructing a first donor minibank of antigen-specific T cell lines can comprise culturing the MNCs in the presence of a plurality of pepmixes. In one embodiment, the MNCs can be PBMCs. In some embodiments, each pepmix from the plurality of pepmixes can comprise a series of overlapping peptides spanning part of or the entire sequence of each antigen.

In some embodiments, each antigen for constructing a first donor minibank of antigen-specific T cell lines can be a tumor associated antigen. In some embodiments, each antigen can be a viral antigen. In some embodiments, at least one antigen for constructing a first donor minibank of antigen-specific T cell lines can be a viral antigen and at least one antigen can be a tumor associated antigen.

In some embodiments, methods as described herein for constructing donor minibanks of antigen specific T cell lines can comprise culturing MNCs from the selected donors in the presence of at least 2 different pepmixes. In some embodiments, methods as described herein can comprise culturing MNCs in the presence of at least 3 different pepmixes. In some embodiments, methods as described herein can comprise culturing MNCs in the presence of at least 4 different pepmixes. In some embodiments, methods as described herein can comprise culturing MNCs in the presence of at least 5 different pepmixes. In some embodiments, methods as described herein can comprise culturing MNCs in the presence of at least 6 different pepmixes. In some embodiments, methods as described herein can comprise culturing MNCs in the presence of at least 7 different pepmixes. In some embodiments, methods as described herein can comprise culturing MNCs in the presence of at least 8 different pepmixes. In some embodiments, methods as described herein can comprise culturing MNCs in the presence of at least 9 different pepmixes. In some embodiments, methods as described herein can comprise culturing MNCs in the presence of at least 10 different pepmixes. In some embodiments, methods as described herein can comprise culturing MNCs in the presence of at least 11 different pepmixes. In some embodiments, methods as described herein can comprise culturing MNCs in the presence of at least 12 different pepmixes. In some embodiments, methods as described herein can comprise culturing MNCs in the presence of at least 13 different pepmixes. In some embodiments, methods as described herein can comprise culturing MNCs in the presence of at least 14 different pepmixes. In some embodiments, methods as described herein can comprise culturing MNCs in the presence of at least 15 different pepmixes. In some embodiments, methods as described herein can comprise culturing MNCs in the presence of at least 16 different pepmixes. In some embodiments, methods as described herein can comprise culturing MNCs in the presence of at least 17 different pepmixes. In some embodiments, methods as described herein can comprise culturing MNCs in the presence of at least 18 different pepmixes. In some embodiments, methods as described herein can comprise culturing MNCs in the presence of at least 19 different pepmixes. In some embodiments, methods as described herein can comprise culturing MNCs in the presence of at least 20 different pepmixes. In some embodiments, methods as described herein can comprise culturing MNCs in the presence of at least more than 20 different pepmixes. In some embodiments, the MNCs can be PBMCs. In some embodiments, each pepmix can comprise a series of overlapping peptides spanning part of an antigen. In some embodiments, each pepmix can comprise a series of overlapping peptides spanning the entire sequence of an antigen.

In some embodiments, methods as described herein for constructing donor minibanks of antigen specific T cell lines can comprise culturing MNCs from the selected donors in the presence of a plurality of pepmixes. In some embodiments, each pepmix can cover at least one antigen that is different than the antigen covered by each of the other pepmixes in the plurality of pepmixes. In some embodiments, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20 different antigens can be covered by the plurality of pepmixes. In some embodiments, at least more than 20 different antigens can be covered by the plurality of pepmixes. In some embodiments, at least one antigen from at least 2 different viruses can be covered by the plurality of pepmixes.

In some embodiments, the antigens used in methods for constructing donor minibanks of antigen specific T cell lines as described herein can be from the EBV (Epstein-Barr virus). In some embodiments, the antigens used in methods as described herein can be from CMV (Cytomegalovirus). In some embodiments, the antigens used in methods as described herein can be from Adenovirus. In some embodiments, the antigens used in methods as described herein can be from BK virus. In some embodiments, the antigens used in methods as described herein can be from JC (John Cunningham virus) virus. In some embodiments, the antigens used in methods as described herein can be from HHV6 (Herpesviruses 6). In some embodiments, the antigens used in methods as described herein can be from HHV8 (Herpesviruses 8). In some embodiments, the antigens used in methods as described herein can be from HBV (Hepatitis B virus). In some embodiments, the antigens used in methods as described herein can be from RSV (Human respiratory syncytial virus). In some embodiments, the antigens used in methods as described herein can be from Influenza. In some embodiments, the antigens used in methods as described herein can be from Parainfluenza. In some embodiments, the antigens used in methods as described herein can be from Bocavirus. In some embodiments, the antigens used in methods as described herein can be from Coronavirus. In some embodiments, the antigens used in methods as described herein can be from LCMV (Lymphocytic choriomeningitis virus). In some embodiments, the antigens used in methods as described herein can be from Mumps. In some embodiments, the antigens used in methods as described herein can be from Measles. In some embodiments, the antigens used in methods as described herein can be from human Metapneumovirus. In some embodiments, the antigens used in methods as described herein can be from Parvovirus B. In some embodiments, the antigens used in methods as described herein can be from Rotavirus. In some embodiments, the antigens used in methods as described herein can be from Merkel cell virus. In some embodiments, the antigens used in methods as described herein can be from herpes simplex virus. In some embodiments, the antigens used in methods as described herein can be from HPV (Human Papillomavirus). In some embodiments, the antigens used in methods as described herein can be from HIV (human immunodeficiency virus). In some embodiments, the antigens used in methods as described herein can be from HTLV1 (Human T-cell leukemia virus, type 1). In some embodiments, the antigens used in methods as described herein can be from West Nile Virus. In some embodiments, the antigens used in methods as described herein can be from Zika virus. In some embodiments, the antigens used in methods as described herein can be from Ebola. In some embodiments, at least one pepmix can cover an antigen from each of RSV, Influenza, Parainfluenza, and HMPV (Human meta-pneumovirus). In some embodiments, the Influenza antigens used in the pepmixes as described herein can be influenza A antigens NP1. In some embodiments, the Influenza antigens used in the pepmixes as described herein can be influenza A MP1. In some embodiments, the Influenza antigens used in the pepmixes as described herein can be influenza A antigens NP1 and MP1. In some embodiments, the RSV antigens used in the pepmixes as described herein can be RSV N proteins. In some embodiments, the RSV antigens used in the pepmixes as described herein can be RSV F proteins. In some embodiments, the RSV antigens used in the pepmixes as described herein can be RSV N proteins and RSV F proteins. In some embodiments, the hMPV antigens used in the pepmixes as described herein can be hMPV F proteins. In some embodiments, the hMPV antigens used in the pepmixes as described herein can be hMPV N proteins. In some embodiments, the hMPV antigens used in the pepmixes as described herein can be hMPV M2-1 proteins. In some embodiments, the hMPV antigens used in the pepmixes as described herein can be hMPV M proteins. In some embodiments, the hMPV antigens used in the pepmixes as described herein can be a combination of hMPV F proteins, hMPV N proteins, hMPV M2-1, and hMPV M proteins. In some embodiments, the PIV antigens used in the pepmixes as described herein can be PIV M proteins. In some embodiments, the PIV antigens used in the pepmixes as described herein can be PIV HN proteins. In some embodiments, the PIV antigens used in the pepmixes as described herein can be PIV N proteins. In some embodiments, the PIV antigens used in the pepmixes as described herein can be PIV F proteins. In some embodiments, the PIV antigens used in the pepmixes as described herein can be a combination of PIV M proteins, PIV HN proteins, PIV N proteins, and PIV F proteins.

In some embodiments, methods as described herein for constructing donor minibanks of antigen specific T cell lines can comprise culturing PBMCs from the selected donors in the presence of pepmixes spanning Influenza A antigen NP1 and Influenza A antigen MP1. In some embodiments, methods as described herein can comprise culturing PBMCs in the presence of pepmixes spanning RSV antigen N and RSV antigen F. In some embodiments, methods as described herein can comprise culturing PBMCs in the presence of pepmixes spanning hMPV antigen F. In some embodiments, methods as described herein can comprise culturing PBMCs in the presence of pepmixes spanning hMPV antigen N. In some embodiments, methods as described herein can comprise culturing PBMCs in the presence of pepmixes spanning hMPV antigen M2-1. In some embodiments, methods as described herein can comprise culturing PBMCs in the presence of pepmixes spanning hMPV antigen M. In some embodiments, methods as described herein can comprise culturing PBMCs in the presence of pepmixes spanning PIV antigen M. In some embodiments, methods as described herein can comprise culturing PBMCs in the presence of pepmixes spanning PIV antigen HN. In some embodiments, methods as described herein can comprise culturing PBMCs in the presence of pepmixes spanning PIV antigen N. In some embodiments, methods as described herein can comprise culturing PBMCs in the presence of pepmixes spanning PIV antigen F.

In some embodiments, methods as described herein for constructing donor minibanks of antigen specific T cell lines can comprise culturing PBMCs from the selected donors in the presence of pepmixes that cover an antigen from each EBV, CMV, adenovirus, BK, and HHV6. In some embodiments, at least one pepmix can cover an antigen from EBV, at least one pepmix can cover an antigen from CMV, at least one pepmix can cover an antigen from adenovirus, at least one pepmix can cover an antigen from BK, and at least one pepmix can cover an antigen from HHV6. In some embodiments, the EBV antigens can be LMP2. In some embodiments, the EBV antigens can be EBNA1. In some embodiments, the EBV antigens can be BZLF1. In some embodiments, the EBV antigens can be a combination of the CMV antigens. In some embodiments, the CMV antigens can be from IE1. In some embodiments, the CMV antigens can be from pp65. In some embodiments, the CMV antigens can be from a combination of IE1 and pp65. In some embodiments, the adenovirus antigens can be from Hexon. In some embodiments, the adenovirus antigens can be from Penton. In some embodiments, the adenovirus antigens can be from a combination of Hexon and Penton. In some embodiments, the BK virus antigens can be from VP1. In some embodiments, the BK virus antigens can be from large T. In some embodiments, the BK virus antigens can be from a combination of VP1 and large T. In some embodiments, the HHV6 antigens can be from U90. In some embodiments, the HHV6 antigens can be from U11. In some embodiments, the HHV6 antigens can be from U14. In some embodiments, the HHV6 antigens can be from a combination of U90, U11, and U14.

In some embodiments, methods as described herein for constructing donor minibanks of antigen specific T cell lines can comprise culturing PBMCs in the presence of pepmixes spanning EBV antigen LMP2. In some embodiments, methods as described herein can comprise culturing PBMCs in the presence of pepmixes spanning EBV antigen EBNA1. In some embodiments, methods as described herein can comprise culturing PBMCs in the presence of pepmixes spanning EBV antigen BZLF1. In some embodiments, methods as described herein can comprise culturing PBMCs in the presence of pepmixes spanning CMV antigen IE1. In some embodiments, methods as described herein can comprise culturing PBMCs in the presence of pepmixes spanning CMV antigen pp65. In some embodiments, methods as described herein can comprise culturing PBMCs in the presence of pepmixes spanning adenovirus antigens Hexon. In some embodiments, methods as described herein can comprise culturing PBMCs in the presence of pepmixes spanning Penton. In some embodiments, methods as described herein can comprise culturing PBMCs in the presence of pepmixes spanning BK virus antigen VP1. In some embodiments, methods as described herein can comprise culturing PBMCs in the presence of pepmixes spanning BK virus antigen large T. In some embodiments, methods as described herein can comprise culturing PBMCs in the presence of pepmixes spanning HHV6 antigen U90. In some embodiments, methods as described herein can comprise culturing PBMCs in the presence of pepmixes spanning HHV6 antigen U11. In some embodiments, methods as described herein can comprise culturing PBMCs in the presence of pepmixes spanning HHV6 antigen U14.

In some embodiments, methods as described herein for constructing donor minibanks of antigen specific T cell lines can comprise culturing PBMCs from the selected donors in the presence of pepmixes that cover an antigen from a coronavirus. In some embodiments, the coronavirus is a β-coronavirus (β-CoV). In some embodiments, the coronavirus is an α-coronavirus (α-CoV). In some the β-CoV is selected from SARS-CoV, MERS-CoV, HCoVHKU1, and HCoV-OC43. In some embodiments, the α-CoV selected from HCoV-E229 and HCoV-NL63. In some embodiments, methods as described herein for constructing donor minibanks of antigen specific T cell lines can comprise culturing PBMCs with a plurality of pepmix libraries, each pepmix library containing a plurality of overlapping peptides spanning all or a portion of a SARS-CoV2 antigen or an antigen from the one or more additional viruses. In some embodiments, the VSTs are generated by contacting T cells with APCs such as DCs primed with a plurality of pepmix libraries, each pepmix library containing a plurality of overlapping peptides spanning all or a portion of a viral antigen, wherein at least one of the plurality of pepmix libraries spans a first antigen from SARS-CoV2 and wherein at least one (or a portion of one) additional pepmix library of the plurality of pepmix libraries spans each second antigen. In some embodiments, the VSTs are generated by contacting T cells with APCs such as DCs nucleofected with at least one DNA plasmid encoding at least one SARS-CoV2 antigen, or a portion thereof, and at least one DNA plasmid encoding each second antigen, or a portion thereof. In some embodiments, the plasmid encodes at least one SARS-CoV2 antigen, or a portion thereof, and at least one of the additional antigens, or a portion thereof. In some embodiments, the VSTs comp1ise CD4+ T lymphocytes and CD8+ T-lymphocytes. In some embodiments, the VSTs express αβ T cell receptors. In some embodiments, the VSTs are MHC-restricted. In some embodiments, the SARS-CoV2 antigen comprises one or more antigens selected from the group consisting of nsp 1; nsp3; nsp4; nsp5; nsp6; nsp7a, nsp8, nsp10; nsp12; nsp13; nsp14; nsp15; and nsp16. In some embodiments, the SARS-CoV2 antigen comprises one or more antigen selected from the group consisting of Spike (S); Envelope protein (E); Matrix protein (M); and Nucleocapsid protein (N). In some embodiments, the SARS-CoV2 antigen comprises one or more antigen selected from the group consisting of SARS-CoV-2 (AP3A); SARS-CoV-2 (NSS); SARS-CoV-2 (ORF1O); SARS-CoV-2 (ORF9B); and SARS-CoV-2 (Y14). In some embodiments, methods as described herein for constructing donor minibanks of antigen specific T cell lines can comprise culturing PBMCs from the selected donors in the presence of pepmixes that cover one or more SARS-CoV2 antigens and one or more additional antigen selected from the group consisting of PIV antigen M, PIV antigen HN, PIV antigen N, PIV antigen F, influenza antigen NP1, influenza antigen MP1, RSV antigen N, RSV antigen F, hMPV antigen M, hMPV antigen M2-1, hMPV antigen F, hMPV antigen N, and AdV antigen Hexon, AdV antigen Penton and combinations thereof. In some embodiments, the additional antigen comprises PIV antigen M, PIV antigen HN, PIV antigen N, PIV antigen F, influenza antigen NP1, influenza antigen MP1, RSV antigen N, RSV antigen F, hMPV antigen M, hMPV antigen M2-1, hMPV antigen F, hMPV antigen N, AdV antigen Hex on, AdV antigen Penton and combinations thereof.

In some embodiments, methods as described herein for constructing donor minibanks of antigen specific T cell lines can comprise culturing PBMCs from the selected donors in the presence of pepmixes that cover an antigen from a hepatitis B virus (HBV). In some embodiments, the HBV antigen is selected from HBV Core antigen, HBV Surface Antigen, and each of HBV Core antigen and HBV Surface Antigen.

In some embodiments, methods as described herein for constructing donor minibanks of antigen specific T cell lines can comprise culturing PBMCs from the selected donors in the presence of pepmixes that cover an antigen from a Human Herpesvirus-8 (HHV-8). In some embodiments, the HHV-8 antigen comprises a latent antigen. In some embodiments the HHV-8 antigen comprises a lytic antigen. In some embodiments, the HHV-8 antigen is selected from LANA-1 (ORF3); LANA-2 (vIRF3, K10.5); vCYC (ORF72); RTA (ORF50); vFLIP (ORF71); Kaposin (ORF12, K12); gB (ORF8); MIR1 (K3); SSB (ORF6); TS (ORF70), and a combination thereof.

In some embodiments, the methods as described herein for constructing donor minibanks of antigen specific T cell lines (e.g., VSTs) comprise culturing antigen specific T cell lines ex vivo in the presence of both IL-7 and IL-4. In some embodiments, the VSTs have expanded sufficiently within 9-18 days of culture such that they are ready for administration to a patient. In some embodiments, the pepmix as described herein can comprise 15 mer peptides. In one embodiment, peptides in the pepmix that span the antigen can overlap in sequence by 11 amino acids. In some embodiments, constructing a first donor minibank of antigen-specific T cell lines can comprise expanding the antigen-specific T cells. In some embodiments, constructing a first donor minibank of antigen-specific T cell lines can comprise testing the antigen specific T cells for antigen-specific cytotoxicity. In some embodiments, minibanks of antigen-specific T cell lines can be produced via the methods of constructing a first donor minibank of antigen-specific T cell lines as disclosed herein. In some embodiments, minibanks of antigen-specific T cell lines can be derived from a plurality of donors selected via methods as described herein. In some embodiments, banks of antigen-specific T cell lines can comprise a plurality of minibanks derived from a plurality of donors selected via methods as described herein.

In embodiments, two or more cell lines of the donor minibanks generated as described by any of the methods provided herein may be pooled together to generate a universal antigen-specific T cell product. In embodiments, two or more cell lines of the donor minibanks generated as described herein may be used as a universal antigen-specific T cell product, e.g., by administration to a patient in a single dosing session.

The present disclosure provides methods of treating a disease or condition by administering to a patient one or more suitable antigen-specific T cell lines from the minibank as described herein (e.g., two or more of such T cell lines), and/or a universal antigen-specific T cell product described herein. In some embodiments, the sole criterion for choosing an antigen-specific T cell line for administration to a patient is that the patient shares at least two HLA alleles with the donor from whom the MNCs used in the manufacture of the antigen-specific T cell line were isolated. In one embodiment, the MNCs can be PBMCs. In some embodiments, a patient may be administered the universal antigen-specific T cell product described herein without prior HLA typing and/or without taking into account the patient's HLA type. In some embodiments, a patient may be administered in a single dosing session two or more of the antigen-specific T cell lines contained in a minibank described herein without prior HLA typing and/or without taking into account the patient's HLA type. In some particular embodiments, a patient may be administered in a single dosing session all of the antigen-specific T cell lines contained in a minibank described herein without prior HLA typing and/or without taking into account the patient's HLA type. In some embodiments, the disease treated can be a viral infection or virus-associated disease. In some embodiments, the disease treated can be a cancer.

In some embodiments, patients being treated by one or more suitable antigen-specific T cell lines from the minibank as described herein (e.g., two or more of such T cell lines) and/or the universal antigen-specific T cell product can be immunocompromised. In some embodiments, the patients are immunocompromised due to a treatment the patients received to treat the disease or condition or another disease or condition. In some embodiments, the patients are immunocompromised due to age. In one embodiment, patients are immunocompromised due to young age. In one embodiment, patients are immunocompromised due to old age. In some embodiments, the condition treated can be an immune deficiency. In one embodiment, the immune deficiency is primary immune deficiency. In some embodiments, the patients are in need of a transplant therapy.

In some embodiments, the transplanted material received by the patients as described herein can comprise stem cells. In some embodiments, the transplanted material received by the patients as described herein can comprise a solid organ. In some embodiments, the solid organ is a kidney. In some embodiments, the transplanted material received by the patients as described herein can comprise bone marrow. In some embodiments, the transplanted material received by the patients as described herein can comprise stem cells, a solid organ, and bone marrow. In some embodiments, the methods comprise administering the first antigen-specific T cell line selected in step (g) as described in the immediately preceding paragraph to the patient.

In some embodiments, the administration to the patients can be for treatment of a viral infection. In some embodiments, the administration to the patients can be for treatment of a tumor. In some embodiments, the administration to the patients can be for primary immune deficiency prior to transplant. In some embodiments, methods as described herein can comprise administering a plurality of antigen-specific T cell lines to the patient, wherein a second antigen-specific T cell line can be selected from the same minibank as the first antigen specific T cell line. In some embodiments, a second antigen-specific T cell line can be selected from a different minibank than the minibank from which the first antigen specific T cell line was obtained. In some embodiments, the second antigen specific T cell line can be selected by repeating the method of selecting a first antigen-specific T cell line from a minibank or from a minibank comprised in the bank as described herein with all remaining antigen-specific T cell lines in the donor bank other than the first antigen specific T cell line. In embodiments, the methods as described herein can comprise administering to the patient a universal antigen-specific T cell product, wherein the product comprises a population of antigen-specific T cells wherein the population of antigen-specific T cells comprises at least 2 cell lines wherein each cell line is generated from separate donors, wherein the HLA type of each donor differs from at least one of the other donors on at least one HLA allele. The universal antigen-specific T cells may be obtained by pooling cell lines from one or more donor minibank and/or may be administered as individual antigen-specific T cells from a donor minibank in a single dosing session.

The present disclosure provides methods of constructing a donor bank made up of a plurality of minibanks of antigen specific T cell lines. In some embodiments, the methods can comprise step A) performing steps (a) through (j) set forth in the method of constructing a first donor minibank of antigen-specific T cell lines as described herein. In some embodiments, a first minibank is constructed. In some embodiments, the methods can comprise step B) repeating steps (a) through (j) set forth in the method of constructing a first donor minibank of antigen-specific T cell lines as described herein. In some embodiments, one or more second rounds can be conducted to construct one or more second minibanks. In some embodiments, prior to starting each second round of the method as described herein, a new donor pool can be generated. In some embodiments, the new donor pool can comprise the first donor pool, less any greatest matched donors removed in accordance with each prior cycle of step (d) from the first and any prior second rounds of the method of constructing a first donor minibank of antigen-specific T cell lines as described herein. In some embodiments, the new donor pool can comprise an entirely new population of potential donors not included in the first donor pool. In some embodiments, the new donor pool can comprise a combination of the new donor pool comprising the first donor pool, less any greatest matched donors removed in accordance with each prior cycle of step (d) from the first and any prior second rounds of the method of constructing a first donor minibank of antigen-specific T cell lines as described herein and an entirely new population of potential donors not included in the first donor pool.

In some embodiments, the methods can comprise reconstituting the first plurality of prospective patients from the first prospective patient population by returning all prospective patients that had been previously removed in accordance with each prior cycle of step (e) set forth in the method of constructing a first donor minibank of antigen-specific T cell lines as described herein from the first and any prior second rounds. In some embodiments, steps (g) through (j) set forth in the method of constructing a first donor minibank of antigen-specific T cell lines as described herein may optionally be performed following each round of the method or they may be performed at any time after step A) as described in the immediately preceding paragraph.

In some embodiments, the culturing of MNCs can be in a vessel comprising a gas permeable culture surface. In one embodiment, the vessel can be an infusion bag with a gas permeable portion. In one embodiment, the vessel can be a rigid vessel. In one embodiment, the vessel can be a GRex bioreactor (Wilson Wolf, St Paul, MN). In some embodiments, culturing the MNCs for constructing a first donor minibank of antigen-specific T cell lines as described herein can be conducted in the presence of one or more cytokine. In one embodiment, the MNCs can be PMBCs. In one embodiment, the cytokine can include IL4. In one embodiment, the cytokine can include IL7. In one embodiment, the cytokine can include IL4 and IL7. In one embodiment, the cytokine can include IL4 and IL7, but not IL2.

In some embodiments, the one or more antigen can be in the form of a whole protein. In some embodiments, the one or more antigen can be in the form of a pepmix comprising a series of overlapping peptides spanning part of or the entire sequence of each antigen. In some embodiments, the one or more antigen can be in the form of a combination of the form of a whole protein and the form of a pepmix comprising a series of overlapping peptides spanning part of or the entire sequence of each antigen. In some embodiments, methods for constructing a donor bank made up of a plurality of minibanks of antigen specific T cell lines can comprise culturing the MNCs in the presence of a plurality of pepmixes. In one embodiment, the MNCs can be PBMCs. In some embodiments, each pepmix from the plurality of pepmixes can comprise a series of overlapping peptides spanning part of or the entire sequence of each antigen. The antigen may be presented on a dendritic cell. The antigen may be directly contacted with the MNCs (e.g., PBMCs) from the donor selected via the method disclosed herein.

In other embodiments, each antigen contacted with the cells can comprise a tumor associated antigen. In other embodiments, each antigen can be a viral antigen. In some embodiments, at least one antigen contacted with the cells can be a viral antigen and at least one antigen contacted with the cells can be a tumor associated antigen.

In some embodiments, methods of constructing a donor bank made up of a plurality of minibanks of antigen specific T cell lines as described herein can comprise culturing MNCs in the presence of at least 2 different pepmixes. In some embodiments, methods as described herein can comprise culturing MNCs in the presence of at least 3 different pepmixes. In some embodiments, methods as described herein can comprise culturing MNCs in the presence of at least 4 different pepmixes. In some embodiments, methods as described herein can comprise culturing MNCs in the presence of at least 5 different pepmixes. In some embodiments, methods as described herein can comprise culturing MNCs in the presence of at least 6 different pepmixes. In some embodiments, methods as described herein can comprise culturing MNCs in the presence of at least 7 different pepmixes. In some embodiments, methods as described herein can comprise culturing MNCs in the presence of at least 8 different pepmixes. In some embodiments, methods as described herein can comprise culturing MNCs in the presence of at least 9 different pepmixes. In some embodiments, methods as described herein can comprise culturing MNCs in the presence of at least 10 different pepmixes. In some embodiments, methods as described herein can comprise culturing MNCs in the presence of at least 11 different pepmixes. In some embodiments, methods as described herein can comprise culturing MNCs in the presence of at least 12 different pepmixes. In some embodiments, methods as described herein can comprise culturing MNCs in the presence of at least 13 different pepmixes. In some embodiments, methods as described herein can comprise culturing MNCs in the presence of at least 14 different pepmixes. In some embodiments, methods as described herein can comprise culturing MNCs in the presence of at least 15 different pepmixes. In some embodiments, methods as described herein can comprise culturing MNCs in the presence of at least 16 different pepmixes. In some embodiments, methods as described herein can comprise culturing MNCs in the presence of at least 17 different pepmixes. In some embodiments, methods as described herein can comprise culturing MNCs in the presence of at least 18 different pepmixes. In some embodiments, methods as described herein can comprise culturing MNCs in the presence of at least 19 different pepmixes. In some embodiments, methods as described herein can comprise culturing MNCs in the presence of at least 20 different pepmixes. In some embodiments, methods as described herein can comprise culturing MNCs in the presence of at least more than 20 different pepmixes. In some embodiments, the MNCs can be PBMCs. In some embodiments, each pepmix can comprise a series of overlapping peptides spanning part of an antigen. In some embodiments, each pepmix can comprise a series of overlapping peptides spanning the entire sequence of an antigen

In some embodiments, methods as described herein can comprise culturing MNCs in the presence of a plurality of pepmixes. In some embodiments, each pepmix can cover at least one antigen that is different than the antigen covered by each of the other pepmixes in the plurality of pepmixes. In some embodiments, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20 different antigens can be covered by the plurality of pepmixes. In some embodiments, at least more than 20 different antigens can be covered by the plurality of pepmixes. In some embodiments, at least one antigen from at least 2 different viruses can be covered by the plurality of pepmixes.

In some embodiments, the antigens used in methods as described herein can be from EBV (Epstein-Barr virus). In some embodiments, the antigens used in methods as described herein can be from CMV (Cytomegalovirus). In some embodiments, the antigens used in methods as described herein can be from Adenovirus. In some embodiments, the antigens used in methods as described herein can be from BK virus. In some embodiments, the antigens used in methods as described herein can be from JC virus (John Cunningham virus). In some embodiments, the antigens used in methods as described herein can be from HHV6 (Herpesviruses 6). In some embodiments, the antigens used in methods as described herein can be from RSV (Human respiratory syncytial virus). In some embodiments, the antigens used in methods as described herein can be from Influenza. In some embodiments, the antigens used in methods as described herein can be from Parainfluenza. In some embodiments, the antigens used in methods as described herein can be from Bocavirus. In some embodiments, the antigens used in methods as described herein can be from Coronavirus. In some embodiments, the antigens used in methods as described herein can be from SARS-CoV2. In some embodiments, the antigens used in methods as described herein can be from LCMV (Lymphocytic choriomeningitis virus). In some embodiments, the antigens used in methods as described herein can be from Mumps. In some embodiments, the antigens used in methods as described herein can be from Measles. In some embodiments, the antigens used in methods as described herein can be from human Metapneumovirus. In some embodiments, the antigens used in methods as described herein can be from Parvovirus B. In some embodiments, the antigens used in methods as described herein can be from Rotavirus. In some embodiments, the antigens used in methods as described herein can be from Merkel cell virus. In some embodiments, the antigens used in methods as described herein can be from herpes simplex virus. In some embodiments, the antigens used in methods as described herein can be from HPV (Human Papillomavirus). In some embodiments, the antigens used in methods as described herein can be from HIV (human immunodeficiency virus). In some embodiments, the antigens used in methods as described herein can be from HTLV1 (Human T-cell leukemia virus, type 1). In some embodiments, the antigens used in methods as described herein can be from HHV8 (Herpesviruses 8). In some embodiments, the antigens used in methods as described herein can be from hepatitis B virus (HBV). In some embodiments, the antigens used in methods as described herein can be from West Nile Virus. In some embodiments, the antigens used in methods as described herein can be from Zika virus. In some embodiments, the antigens used in methods as described herein can be from Ebola.

In some embodiments, at least one pepmix can cover an antigen from each of RSV, Influenza, Parainfluenza, and HMPV (Human meta-pneumovirus). In some embodiments, the Influenza antigens used in the pepmixes as described herein can be influenza A antigens NP1. In some embodiments, the Influenza antigens used in the pepmixes as described herein can be influenza A MP1. In some embodiments, the Influenza antigens used in the pepmixes as described herein can be influenza A influenza A antigens NP1 and influenza A MP1. In some embodiments, the RSV antigens used in the pepmixes as described herein can be RSV N proteins. In some embodiments, the RSV antigens used in the pepmixes as described herein can be RSV F proteins. In some embodiments, the RSV antigens used in the pepmixes as described herein can be RSV N proteins and RSV F proteins. In some embodiments, the hMPV antigens used in the pepmixes as described herein can be hMPV F proteins. In some embodiments, the hMPV antigens used in the pepmixes as described herein can be hMPV N proteins. In some embodiments, the hMPV antigens used in the pepmixes as described herein can be hMPV M2-1 proteins. In some embodiments, the hMPV antigens used in the pepmixes as described herein can be hMPV M proteins. In some embodiments, the hMPV antigens used in the pepmixes as described herein can be a combination of hMPV F proteins, hMPV N proteins, hMPV M2-1, and hMPV M proteins. In some embodiments, the PIV antigens used in the pepmixes as described herein can be PIV M proteins. In some embodiments, the PIV antigens used in the pepmixes as described herein can be PIV HN proteins. In some embodiments, the PIV antigens used in the pepmixes as described herein can be PIV N proteins. In some embodiments, the PIV antigens used in the pepmixes as described herein can be PIV F proteins. In some embodiments, the PIV antigens used in the pepmixes as described herein can be a combination of PIV M proteins, PIV HN proteins, PIV N proteins, and PIV F proteins.

In some embodiments, methods as described herein can comprise culturing MNCs or PBMCs in the presence of pepmixes spanning Influenza A antigen NP1 and Influenza A antigen MP1. In some embodiments, methods as described herein can comprise culturing in the presence of pepmixes spanning RSV antigen N and RSV antigen F. In some embodiments, methods as described herein can comprise culturing in the presence of pepmixes spanning hMPV antigen F. In some embodiments, methods as described herein can comprise culturing in the presence of pepmixes spanning hMPV antigen N. In some embodiments, methods as described herein can comprise culturing in the presence of pepmixes spanning hMPV antigen M2-1. In some embodiments, methods as described herein can comprise culturing in the presence of pepmixes spanning hMPV antigen M. In some embodiments, methods as described herein can comprise culturing in the presence of pepmixes spanning PIV antigen M. In some embodiments, methods as described herein can comprise culturing in the presence of pepmixes spanning PIV antigen HN. In some embodiments, methods as described herein can comprise culturing in the presence of pepmixes spanning PIV antigen N. In some embodiments, methods as described herein can comprise culturing in the presence of pepmixes spanning PIV antigen F.

In some embodiments, methods as described herein can comprise culturing MNCs or PBMCs in the presence of pepmixes spanning Influenza A antigen NP1 and Influenza A antigen MP1. In some embodiments, methods as described herein can comprise culturing in the presence of pepmixes spanning RSV antigen N and RSV antigen F. In some embodiments, methods as described herein can comprise culturing in the presence of pepmixes spanning hMPV antigen F. In some embodiments, methods as described herein can comprise culturing in the presence of pepmixes spanning hMPV antigen N. In some embodiments, methods as described herein can comprise culturing in the presence of pepmixes spanning hMPV antigen M2-1. In some embodiments, methods as described herein can comprise culturing in the presence of pepmixes spanning hMPV antigen M. In some embodiments, methods as described herein can comprise culturing in the presence of pepmixes spanning PIV antigen M. In some embodiments, methods as described herein can comprise culturing in the presence of pepmixes spanning PIV antigen HN. In some embodiments, methods as described herein can comprise culturing in the presence of pepmixes spanning PIV antigen N. In some embodiments, methods as described herein can comprise culturing in the presence of pepmixes spanning PIV antigen F.

In some embodiments, at least one pepmix as described herein can cover an antigen from EBV, CMV, adenovirus, BK, and HHV6. In some embodiments, the EBV antigen can be LMP2. In some embodiments, the EBV antigen can be EBNA1. In some embodiments, the EBV antigen can be BZLF1. In some embodiments, the EBV antigen can be LMP2, EBNA1, and BZLF1. In some embodiments, the CMV antigen can be IE1. In some embodiments, the CMV antigen can be pp65. In some embodiments, the CMV antigen can be IE1 and pp65.

In some embodiments, the adenovirus antigens can be Hexon. In some embodiments, the adenovirus antigens can be Penton. In some embodiments, the adenovirus antigens can be Hexon and Penton. In some embodiments, the BK virus antigen can be VP1. In some embodiments, the BK virus antigen can be large T. In some embodiments, the BK virus antigen can be VP1 and large T. In some embodiments, the HHV6 antigen can be U90. In some embodiments, the HHV6 antigen can be U11. In some embodiments, the HHV6 antigen can be U14. In some embodiments, the HHV6 antigen can be U90, U11, and U14.

In some embodiments, methods as described herein can comprise culturing MNCs or PBMCs in the presence of pepmixes spanning EBV antigen LMP2, EBV antigen EBNA1, and EBV antigen BZLF1. In some embodiments, methods as described herein can comprise culturing in the presence of pepmixes spanning CMV antigen IE1 and CMV antigen pp65. In some embodiments, methods as described herein can comprise culturing in the presence of pepmixes spanning adenovirus antigens Hexon and adenovirus antigens Penton. In some embodiments, methods as described herein can comprise culturing in the presence of pepmixes spanning BK virus antigen VP1 and large T. In some embodiments, methods as described herein can comprise culturing in the presence of pepmixes spanning HHV6 antigen U90, HHV6 antigen U11, and HHV6 antigen U14.

In some embodiments, methods as described herein can comprise culturing MNCs or PBMCs in the presence of pepmixes spanning HBV Core antigen, HBV Surface Antigen, and each of HBV Core antigen and HBV Surface Antigen.

In some embodiments, methods as described herein can comprise culturing MNCs or PBMCs in the presence of pepmixes spanning an HHV-8 antigen selected from LANA-1 (ORF3); LANA-2 (vIRF3, K10.5); vCYC (ORF72); RTA (ORF50); vFLIP (ORF71); Kaposin (ORF12, K12); gB (ORF8); MIR1 (K3); SSB (ORF6); TS(ORF70), and a combination thereof.

In some embodiments, the pepmix as described herein can comprise 15 mer peptides. In one embodiment, peptides in the pepmix that span the antigen can overlap in sequence by 11 amino acids. In some embodiments, constructing a first donor minibank of antigen-specific T cell lines can comprise expanding the antigen-specific T cells. In some embodiments, constructing a first donor minibank of antigen-specific T cell lines can comprise testing the antigen specific T cells for antigen-specific cytotoxicity.

The present disclosure provides donor banks that can comprise a plurality of minibanks of antigen-specific T cell lines. In some embodiments, the donor bank can be produced via the method of constructing a donor bank made up of a plurality of minibanks of antigen specific T cell lines. The present disclosure provides methods of treating a disease or condition comprising administering to a patient one or more suitable antigen-specific T cell lines from the donor bank as described herein.

The present disclosure provides methods of treating a disease or condition by administering to a patient one or more suitable antigen-specific T cell lines from the donor bank as described herein (e.g., two or more suitable antigen specific T cell lines) and/or a universal antigen-specific T cell product described herein. In some embodiments, the sole criteria for administration of the antigen-specific T cell line to the patient is that the patient shares at least two HLA alleles with the donor from whom the MNCs used in the manufacture of the antigen-specific T cell line were isolated. In one embodiment, the MNCs can be PBMCs. In some embodiments, a patient is administered the universal antigen-specific T cell product described herein. In such embodiments, the patient is treated without prior HLA typing and/or without taking into account the patient's HLA type. In some embodiments, the disease treated can be a viral infection. In some embodiments, the disease treated can be a cancer. In some embodiments, patients being treated by one or more suitable antigen-specific T cell lines from the donor bank as described herein can be immunocompromised. In some embodiments, patients being treated by a universal antigen-specific T cell product described herein can be immunocompromised. In some embodiments, the patients are immunocompromised due to a treatment the patients received to treat the disease or condition or another disease or condition. In some embodiments, the patients are immunocompromised due to age. In one embodiment, patients are immunocompromised due to young age. In one embodiment, patients are immunocompromised due to old age. In some embodiments, the condition treated can be an immune deficiency. In one embodiment, the immune deficiency is primary immune deficiency. In some embodiments, the patients are in need of a transplant therapy

In some embodiments, the transplanted material received by the patients as described herein can comprise stem cells. In some embodiments, the transplanted material received by the patients as described herein can comprise a solid organ. In some embodiments, the solid organ is a kidney. In some embodiments, the transplanted material received by the patients as described herein can comprise bone marrow. In some embodiments, the transplanted material received by the patients as described herein can comprise stem cells, a solid organ, and bone marrow.

In some embodiments, administering the plurality of antigen-specific T cell lines and/or universal antigen-specific T cell product does not result in or exacerbate pre-existing Graft versus host disease (GVHD). In some embodiments, administering plurality of antigen-specific T cell lines and/or universal antigen-specific T cell product can be for treatment of a viral infection. In some embodiments, administering the plurality of antigen-specific T cell lines and/or universal antigen-specific T cell product can be for treatment of a tumor. In some embodiments, administering the plurality of antigen-specific T cell lines and/or universal antigen-specific T cell product can be for primary immune deficiency prior to transplant. In some embodiments, the methods can comprise administering a first and a second antigen-specific T cell line to the patient in a single dosing session. In some embodiments, the second antigen-specific T cell line can be selected from the same donor bank as the first antigen specific T cell line. In some embodiments, the second antigen-specific T cell line can be selected from a different donor minibank than the first antigen specific T cell line. In some embodiments, the second antigen specific T cell line can be administered to the patient in the same dosing session. In some embodiments, the methods can comprise administering a plurality of antigen-specific T cell lines to the patient. In some embodiments, a plurality of the antigen specific T cell lines comprises all of the antigen-specific T cell lines in a donor minibank. In some embodiments, the second antigen specific T cell line can be administered to the patient in the same dosing session.

In some embodiments, the treatment efficacy can be measured based on viremic resolution of infection from the patient. In some embodiments, the treatment efficacy can be measured based on viruric resolution of infection from the patient. In some embodiments, the treatment efficacy can be measured based on resolution of viral load in a sample from the patient. In some embodiments, the treatment efficacy can be measured based on viremic resolution of infection, viruric resolution of infection, and resolution of viral load in a sample from the patient. In some embodiments, the treatment efficacy can be measured post-administration of the antigen specific T cell line.

In some embodiments, the sample can be selected from a tissue sample from the patient. In some embodiments, the sample can be selected from a fluid sample from the patient. In some embodiments, the sample can be selected from cerebral spinal fluid (CSF) from the patient. In some embodiments, the sample can be selected from Bronchoalveolar lavage (BAL) from the patient. In some embodiments, the sample can be selected from stool from the patient. In some embodiments, the sample can be selected from a tissue sample, a fluid sample, CSF, BAL, and stool from the patient.

In some embodiments, the treatment efficacy can be measured by monitoring viral load detectable in the peripheral blood of the patient. In some embodiments, the treatment efficacy can comprise resolution of macroscopic hematuria. In some embodiments, the treatment efficacy can comprise reduction of hemorrhagic cystitis symptoms as measured by the CTCAE-PRO or similar assessment tool that examines patient and/or clinician-reported outcomes. In some embodiments, the treatment efficacy is against a cancer. In some embodiments, the treatment efficacy can be measured based on tumor size reduction post-administration of the antigen specific T cell line. In some embodiments, the treatment efficacy can be measured by monitoring markers of disease burden. In some embodiments, the treatment efficacy can be measured by monitoring tumor lysis detectable in the peripheral blood/serum of the patient. In some embodiments, the treatment efficacy can be measured by monitoring markers of disease burden and tumor lysis detectable in the peripheral blood/serum of the patient. In some embodiments, the treatment efficacy can be measured by monitoring tumor status via imaging studies. In other embodiments, the treatment efficacy can be measured by monitoring a combination of markers of disease burden, tumor lysis detectable in the peripheral blood/serum of the patient, and tumor status via imaging studies.

In some embodiments, an inflammatory response can be detected by observing one or more symptom or sign. In some embodiments, the one or more symptom or sign can include constitutional symptoms. In some embodiments, the constitutional symptoms can be fever, rigors, headache, malaise, fatigue, nausea, vomiting, or arthralgia. In some embodiments, the one or more symptom or sign can include vascular symptoms including hypotension. In some embodiments, the one or more symptom or sign can include cardiac symptoms. In one embodiment, cardiac symptoms is arrhythmia. In some embodiments, the one or more symptom or sign can include respiratory compromise. In some embodiments, the one or more symptom or sign can include renal symptoms. In one embodiment, the renal symptom is kidney failure. In one embodiment, the renal symptom is uremia. In some embodiments, the one or more symptom or sign can include laboratory symptoms. In one embodiment, the laboratory symptoms can be coagulopathy and a hemophagocytic lymphohistiocytosis-like syndrome.

The present disclosure provides methods of identifying suitable donors for use in constructing a first donor minibank of antigen-specific T cells. The present disclosure provides methods of constructing a first donor minibank of antigen-specific T cell lines. In some embodiments, the methods can comprise step (a) determining or having determined the HLA type of each of a first plurality of potential donors from a first donor pool. In some embodiments, the methods can comprise step (b) determining or having determined the HLA type of each of a first plurality of prospective patients from a first prospective patient population. In some embodiments, the methods can comprise step (c) comparing the HLA type of each of a first plurality of potential donors from a first donor pool with each of a first plurality of prospective patients from a first prospective patient population. In some embodiments, the methods can comprise step (d) determining, based on the comparison in step (d) as described in this paragraph, a first greatest matched donor, defined as the donor from the first donor pool that has 2 or more allele matches with the greatest number of patients in the first plurality of prospective patients.

In some embodiments, the methods can comprise step (e) selecting the first greatest matched donor for inclusion in a first donor minibank. In some embodiments, the methods can comprise step (f) removing from the first donor pool the first greatest matched donor thereby generating a second donor pool consisting of each of the first plurality of potential donors from the first donor pool except for the first greatest matched donor. In some embodiments, the methods can comprise step (g) removing from the first plurality of prospective patients each prospective patient that has 2 or more allele matches with the first greatest matched donor. In some embodiments, step (g) can comprise generating a second plurality of prospective patients consisting of each of the first plurality of prospective patients except for each prospective patient that has 2 or more allele matches with the first greatest matched donor.

In some embodiments, the methods can comprise step (h) repeating steps (c) through (g) one or more additional times with all donors and prospective patients that have not already been removed in accordance with steps (f) and (g). In some embodiments, each time an additional greatest matched donor is selected in accordance with step (e) that additional greatest matched donor is removed from their respective donor pool in accordance with step (f). In some embodiments, each time a subsequent greatest matched donor is removed from their respective donor pool, each prospective patient that has 2 or more allele matches with that subsequent greatest matched donor is removed from their respective plurality of prospective patients in accordance with step (g). In some embodiments, step (h) sequentially increases the number of selected greatest matched donors in the first donor minibank by 1 following each cycle of the method. In some embodiments, step (h) can comprise depleting the number of the plurality of prospective patients in the patient population following each cycle of the method in accordance with their HLA matching to the selected greatest matched donors. In some embodiments, steps (c) through (g) can be repeated until a desired percentage of the first prospective patient population remains in the plurality of prospective patients. In some embodiments, steps (c) through (g) can be repeated until no donors remain in the donor pool.

In some embodiments, the present disclosure provides administering to a patient a universal antigen-specific T cell product or one or more suitable antigen-specific T cell lines from the donor minibank or the donor bank made of a plurality of the donor minibanks that comprise a plurality of viral antigens including at least one first antigen from parainfluenza virus type 3 (PIV) and at least one second antigen from one or more second virus. In some embodiments, the at least one second antigen is respiratory syncytial virus (RSV). In some embodiments, the at least one second antigen is influenza. In some embodiments, the at least one second antigen is human metapneumovirus (hMPV).

### BRIEF DESCRIPTION OF THE DRAWINGS

FIG. 1 represents the general overview of the selection process of donor banks for use in a patient with a refractory viral infection. Abbreviations: HLA: The human leukocyte antigen. HSCT: Hematopoietic stem cell transplant.

FIG. 2 represents part of the donor selection process. Each donor is compared with patient population to identify the donor who accommodates the majority of patients with a antigen-specific T cell lines based on HLA matching, with a 2-allele minimum threshold.

FIG. 3 represents part of the donor selection process. The donor who accommodates the majority of patients is (i) shortlisted for antigen-specific T cell lines production; (ii) removed from the general donor pool; and (iii) all patients accommodated by this donor are removed from the patient population.

FIG. 4 represents part of the donor selection process. The same step as described in FIG. 2 is repeated identifying the donor who best covers the remaining patients and, then remove both the donor and accommodated patients from further consideration.

FIG. 5 represents part of the donor selection process. The same step as described in FIG. 3 is repeated identifying the donor who best covers the remaining patients and, then remove both the donor and accommodated patients from further consideration.

FIG. 6 represents part of the donor selection process. The same step as described in FIG. 2 is repeated identifying the donor who best covers the remaining patients and, then remove both the donor and accommodated patients from further consideration.

FIG. 7 represents part of the donor selection process. The same step as described in FIG. 3 is repeated identifying the donor who best covers the remaining patients and, then remove both the donor and accommodated patients from further consideration.

FIG. 8 represents part of the donor selection process. The same step as described in FIG. 2 is repeated identifying the donor who best covers the remaining patients and, then remove both the donor and accommodated patients from further consideration.

FIG. 9 represents part of the donor selection process. The same step as described in FIG. 3 is repeated identifying the donor who best covers the remaining patients and, then remove both the donor and accommodated patients from further consideration.

FIG. 10 shows the generation of a mini-bank (comprising donors 2, 3, 5, and 6) that covers at least 95% of the patients (only patients m and k are not matched).

FIG. 11 shows a general manufacturing concepts of the antigen-specific T cell lines.

FIG. 12 shows a flowchart of manufacturing of the antigen-specific T cell lines.

FIG. 13 shows potency of antigen-specific T cell lines against Adv, CMV, EBV, BKV, and HHV6, as assessed using IFN-γ ELISPOT assay.

FIG. 14 shows defining a potency threshold to discriminate potent and non-potent antigen-specific T cell lines against Adv, CMV, EBV, BKV, and HHV6.

FIG. 15 shows correlating the potency of antigen-specific T cell lines with clinical benefit in 20 patients with BK-HC who were successful treated with potent antigen-specific T cell lines. The lack of potency of the T cell lines correlates to the increase of the BK virus concentrations in the patients post-treatments.

FIG. 16 shows the correlation of the use of the antigen-specific T cell lines that are above the potency threshold with the clinical benefits against the BK virus, which shows a general decrease of the level of the BK virus post-treatment.

FIGS. 17A-17D Characteristics of generated CMVST lines and degree of matching with screened subjects (FIG. 17A) T cell expansion of CMVSTs achieved over a 20-day period based on cell counting using trypan blue exclusion. (n=8). (FIG. 17B) Phenotype of the expanded CMVST lines on the day of cryopreservation (mean±SEM, n=8) and (FIG. 17C) frequency of antigen-specific T cells as determined by IFN-7 ELISpot assay after overnight stimulation of CMVSTs with IE1 and pp65 antigen-spanning pepmixes. Results are reported as spot forming cells (SFC) per 2×10⁵ VSTs plated. CMVST lines with a total of ≥30 SFC/2×10⁵ were considered to be positive. (n=8). (FIG. 17D) Number of matching HLA antigens (of 8 total) of CMVST lines identified for clinical use with recipient HLA of screened patients (n=29).

FIG. 18 Treatment outcomes in individual patients infected with cytomegalovirus (CMV). Depiction of plasma CMV viral loads (IU/mL) in patients 2 weeks prior to (viral load level closest to week −2), immediately before (pre) and after (post) infusion (weeks 2, 4 and 6) of CMVSTs. Arrows indicate infusion timepoints.

FIGS. 19A and 19B Frequency of CMV specific T cells in vivo. (FIG. 19A) Frequency of CMVSTs in the peripheral blood before (pre) and after (post) infusion, as measured by IFN-7 ELISpot assay after overnight stimulation with IE1 and pp65 viral pepmixes. Results are expressed as spot-forming cells (SFCs) per 5×10⁵ input cells (mean±SEM, n=10). (FIG. 19B) Persistence of infused CMVSTs in individual patients. Frequency of T cells in peripheral blood as measured by IFN-7 ELISpot assay after stimulation with epitope-specific CMV peptides with restriction to HLA antigens exclusive to the CMVST line or shared between the recipient and the CMVST line.

FIGS. 20A-20D shows an example of the generation of polyclonal multi-R-VSTs from healthy donors. (FIG. 20A) shows a schematic of the multi-R-VST generation protocol. (FIG. 20B) shows the fold expansion achieved over a 10-13 day period based on cell counting using trypan blue exclusion (n=12). (FIG. 20C) and (FIG. 20D) show the phenotype of the expanded cells (mean±SEM, n=12).

FIG. 21 shows Minimal detection of regulatory T cells (Tregs; CD4+CD25+FoxP3+) within the expanded CD4+ T cell populations (mean±SEM, n=8).

FIGS. 22A-22D shows the specificity and enrichment of multi-R-VSTs. (FIG. 22A) shows the specificity of virus-reactive T cells within the expanded T cell lines following exposure to individual stimulating antigens from each of the target viruses. Data is presented as mean±SEM SFC/2×10⁵ (n=12). (FIG. 22B) represents fold enrichment of specificity (PBMC vs multi-R-VST; n=12). (FIG. 22C) shows IFNy production, as assessed by ICS from CD4 helper (top) and CD8 cytotoxic T cells (bottom) after viral stimulation in 1 representative donor (dot plots were gated on CD3+ cells), while (FIG. 22D) shows summary results for 9 donors screened (mean±SEM).

FIG. 23 shows the number of donor-derived VST lines responding to individual stimulating antigens (Influenza, RSV, hMPV, and PIV).

FIG. 24 shows the specificity of virus-reactive T cells within expanded T cell lines following exposure to titrated concentrations of pooled stimulating antigens from each of the target viruses. Data is presented as mean±SEM SFC/2×10⁵ (n=7).

FIG. 25 shows the frequency of CARV-specific T cells in the peripheral blood of healthy donors following exposure to individual stimulating antigens from each of the target viruses. Data is presented as mean±SEM SFC/5×10⁵ (n=12).

FIG. 26 shows peripheral blood CARV-specific precursors are primarily detected within the CD4+ compartment. Shown here is the frequency of CARV-specific T cells in magnetically sorted CD4+ and CD8+ T cell populations isolated from the peripheral blood of healthy donors following exposure to individual stimulating antigens from each of the target viruses. Data is presented as mean±SEM SFC/5×10⁵ (n=4).

FIGS. 27A-27D shows that multi-R-VSTs are polyclonal and polyfunctional. (FIG. 27A) shows dual IFNy and TNFa production from CD3+ T cells as assessed by ICS in 1 representative donor, while (FIG. 27B) shows summary results from 9 donors screened (mean±SEM). (FIG. 27C) shows the cytokine profile of multi-R-VSTs as measured by multiplex bead array. (FIG. 27D) assesses the production of Granzyme B by Ellspot assay. Results are reported as SFC/2×10⁵ input VSTs (mean±SEM, n=9).

FIGS. 28A and 28B shows multi-R-VSTs are exclusively reactive against virus-infected targets. (FIG. 28A) illustrates the cytolytic potential of multi-R-VSTs evaluated by standard 4-hour Cr51 release assay using autologous pepmix-pulsed PHA blasts as targets (E:T 40:1; n=8) with unloaded PHA blasts as a control. Results are presented as percentage of specific lysis (mean±SEM). (FIG. 28B) demonstrates that multi-R-VSTs show no activity against either non-infected autologous or allogeneic PHA blasts, as assessed by Cr⁵¹ release assay.

FIG. 29 shows cytotoxic activity of multi-R-VSTs evaluated by standard 4-hour Cr⁵¹ release assay using autologous pepmix-pulsed PHA blasts as targets (E:T 40:1, 20:1, 10:1, 5:1) with unloaded PHA blasts as a control. Results are presented as percentage of specific lysis (mean±SEM, n=8).

FIGS. 30A-30C shows the detection of RSV- and hMPV-specific T cells in the peripheral blood of HSCT recipients. PBMCs isolated from 2 HSCT recipients with 3 infections were tested for specificity against the infecting viruses, using IFNy Ellspot as a readout. (FIG. 30A) and (FIG. 30B) show results from 2 patients with RSV-associated URTls which were controlled, coincident with a detectable rise in endogenous RSV-specific T cells, while (FIG. 30C) shows clearance of an hMPV-LRTI with expansion of endogenous hMPV-specific T cells. ALC: absolute lymphocyte count.

FIGS. 31A-31C shows the detection of RSV- and PIV (also referred to herein as PIV-3)-specific T cells in the peripheral blood of HSCT recipients. PBMCs isolated from 3 HSCT recipients with 3 infections were tested for specificity against the infecting viruses, using IFNy Ellspot as a readout. (FIG. 31A) and (FIG. 31B) show results from 2 patients with RSV- and PIV-associated URTls and LRTls which were controlled, coincident with a detectable rise in endogenous virus-specific T cells. (FIG. 31C) shows results from a patient with an ongoing PIV-related severe URTI who failed to mount a T cell response against the virus. ALC: absolute lymphocyte count.

FIG. 32 shows HLA Match of Viralym-M Lines Identified in Simulation for Clinical Use in POC Study with 54 prospective patients.

FIG. 33 shows HLA Match of Viralym-M Lines Identified in Simulation for Clinical Use in treating the entire >650 allogeneic HSCT patient population at Baylor's Center for Cell and Gene Therapy.

FIG. 34 shows the lack of alloreactivity of multivirus-specific T cells (Viralym-M cells) as assessed by measuring their cytotoxic activity against HLA-mismatched targets.

FIG. 35 shows the relationship between overall response and degree of HLA match. CR: complete response; PR: partial response; NR: non-responder.

FIG. 36 shows the degree of HLA matching on HLA-Class I, II, or both Class I and Class II across the clinical trial patient population.

FIG. 37 shows overall responses at week 12 based on HLA-matched Alleles (HLA-Class I, II, or both Class I and Class II)

FIG. 38 shows the percent of patients with resolved BK-HC 2, 4, and 6 weeks after receiving VSTs, compared to 33 pediatric allogeneic HSCT patients in a natural history study (Natural History patients), who had BK-HC and received just standard of care treatment for their disease.

FIG. 39 shows the average cystitis grade over time in patients that received VSTs in either a low level HLA-match setting (HLA 1-2/6) or higher level HLA-match setting (HLA 3-4/6).

FIG. 40 is a schematic picture comparing the prior process of manufacturing, banking, and using individual donor cell lines vs. a new process for generating and using a universal donor cell line.

FIG. 41 shows the percent of auto-reactivity of UVSTs against donor PHAs and allo-reactivity against PHA blasts from an unrelated donor.

### DETAILED DESCRIPTION OF THE INVENTION

The details of the invention are set forth in the accompanying description below. Although methods and materials similar or equivalent to those described herein can be used in the practice or testing of the present invention, illustrative methods and materials are now described. Other features, objects, and advantages of the invention will be apparent from the description and from the claims. In the specification and the appended claims, the singular forms also include the plural unless the context clearly dictates otherwise. Unless defined otherwise, all technical and scientific terms used herein have the same meaning as commonly understood by one of ordinary skill in the art to which this invention belongs. All patents and publications cited in this specification are incorporated herein by reference in their entireties.

### General Methods

The practice of the present invention will employ, unless otherwise indicated, conventional techniques of cell culturing, molecular biology (including recombinant techniques), microbiology, cell biology, biochemistry and immunology, which are within the skill of the art. Such techniques are explained fully in the literature, such as, Molecular Cloning: A Laboratory Manual, third edition (Sambrook et al., 2001) Cold Spring Harbor Press; Oligonucleotide Synthesis (P. Herdewijn, ed., 2004); Animal Cell Culture (R. I. Freshney), ed., 1987); Methods in Enzymology (Academic Press, Inc.); Handbook of Experimental Immunology (D. M. Weir & C. C. Blackwell, eds.); Gene Transfer Vectors for Mammalian Cells (J. M. Miller & M. P. Calos, eds., 1987); Current Protocols in Molecular Biology (F. M. Ausubel et al., eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Manual of Clinical Laboratory Immunology (B. Detrick, N. R. Rose, and J. D. Folds eds., 2006); Immunochemical Protocols (J. Pound, ed., 2003); Lab Manual in Biochemistry: Immunology and Biotechnology (A. Nigam and A. Ayyagari, eds. 2007); Immunology Methods Manual: The Comprehensive Sourcebook of Techniques (Ivan Lefkovits, ed., 1996); Using Antibodies: A Laboratory Manual (E. Harlow and D. Lane, eds., 1988); and others.

### Definitions

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

As used herein, the use of the word “a” or “an” when used in conjunction with the term “comprising” in the claims and/or the specification may mean “one,” but it is also consistent with the meaning of “one or more,” “at least one,” and “one or more than one.” By way of example, “an element” means one element or more than one element. Some embodiments of the invention may consist of or consist essentially of one or more elements, method steps, and/or methods of the invention. It is contemplated that any method or composition described herein can be implemented with respect to any other method or composition described herein.

The term “about” when immediately preceding a numerical value means±0% to 10% of the numerical value, ±0% to 10%, ±0% to 9%, ±0% to 8%, ±0% to 7%, ±0% to 6%, ±0% to 5%, ±0% to 4%, ±0% to 3%, ±0% to 2%, ±0% to 1%, ±0% to less than 1%, or any other value or range of values therein. For example, “about 40” means±0% to 10% of 40 (i.e., from 36 to 44).

The term “and/or” is used in this disclosure to mean either “and” or “or” unless indicated otherwise.

Throughout this specification, unless the context requires otherwise, the words “comprise,” “comprises,” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

The term “disorder” is used in this disclosure to mean, and is used interchangeably with, the terms disease, condition, or illness, unless otherwise indicated.

An “effective amount” when used in connection with a therapeutic agent (e.g., an antigen specific T cell product or cell line disclosed herein) is an amount effective for treating or preventing a disease or disorder in a subject as described herein.

The term “e.g.” is used herein to mean “for example,” and will be understood to imply the inclusion of a stated step or element or group of steps or elements but not the exclusion of any other step or element or group of steps or elements.

By “optional” or “optionally,” it is meant that the subsequently described event or circumstance may or may not occur, and that the description includes instances where the event or circumstance occurs and instances in which it does not.

The term “tumor associated antigen” as used herein refers to an antigenic substance produced/expressed on tumor cells and which triggers an immune response in the host.

Exemplary tumor antigens include at least the following: carcinoembryonic antigen (CEA) for bowel cancers; CA-125 for ovarian cancer; MUC-1 or epithelial tumor antigen (ETA) or CA15-3 for breast cancer; tyrosinase or melanoma-associated antigen (MAGE) for malignant melanoma; and abnormal products of ras, p53 for a variety of types of tumors; alphafetoprotein for hepatoma, ovarian, or testicular cancer; beta subunit of hCG for men with testicular cancer; prostate specific antigen for prostate cancer; beta 2 microglobulin for multiple myelom and in some lymphomas; CA19-9 for colorectal, bile duct, and pancreatic cancer; chromogranin A for lung and prostate cancer; TA90 for melanoma, soft tissue sarcomas, and breast, colon, and lung cancer. Examples of tumor antigens are known in the art, for example in Cheever et al., 2009, which is incorporated by reference herein in its entirety.

Specific examples of tumor antigens include at least CEA, MHC, CTLA-4, gp100, mesothelin, PD-L1, TRP1, CD40, EGFP, Her2, TCR alpha, trp2, TCR, MUC1, cdr2, ras, 4-1BB, CT26, GITR, OX40, TGF-α. WT1, MUC1, LMP2, HPV E6 E7, EGFRvIII, HER-2/neu, MAGE A3, p53 nonmutant, NY-ESO-1, PSMA, GD2, Melan A/MART1, Ras mutant, gp 100, p53 mutant, Proteinase3 (PR1), bcr-abl, Tyrosinase, Survivin, PSA, hTERT, EphA2, PAP, ML-IAP, AFP, EpCAM, ERG (TMPRSS2 ETS fusion gene), NA17, PAX3, ALK, Androgen receptor, Cyclin B1, Polysialic acid, MYCN, RhoC, TRP-2, GD3, Fucosyl GM1, Mesothelin, PSCA, MAGE A1, sLe(a), CYP1B1, PLAC1, GM3, BORIS, Tn, GloboH, ETV6-AML, NY-BR-1, RGS5, SART3, STn, Carbonic anhydrase IX, PAX5, OY-TES1, Sperm protein 17, LCK, HMWMAA, AKAP-4, SSX2, XAGE 1, B7H3, Legumain, Tie 2, Page4, VEGFR2, MAD-CT-1, FAP, PDGFR-β, MAD-CT-2, and Fos-related antigen 1, for example.

The term “viral antigen” as used herein refers to an antigen that is protein in nature and is closely associated with the virus particle. In specific embodiments, a viral antigen is a coat protein.

Specific examples of viral antigen include at least a virus selected from EBV, CMV, Adenovirus, BK, JC virus, HHV6, RSV, Influenza, Parainfluenza, Bocavirus, Coronavirus, LCMV, Mumps, Measles, human Metapneumovirus, Parvovirus B, Rotavirus, Merkel cell virus, herpes simplex virus, HPV, HBV, HIV, HTLV1, HHV8 and West Nile Virus, zika virus, Ebola.

The term “virus-specific T cells” or “VSTs” or “virus-specific T cell lines” or “VST cell lines” are used interchangeably herein to refer to T cell lines, e.g., as described herein, that have been expanded and/or manufactured outside of a subject and that have specificity and potency against a virus or viruses of interest. The VSTs may be monoclonal or oligoclonal, in some embodiments. In particular embodiments the VSTs are polyclonal. As described herein, in some embodiments, a viral antigen or several viral antigens are presented to native T cells or memory T cells in peripheral blood mononuclear cells and the native CD4+ and/or CD8+ T cell populations with specificity for the viral antigens(s) expand in response. For example, a virus-specific T cell for EBV in a sample of PBMCs obtained from a suitable donor can recognize (bind to) an EBV antigen (e.g., a peptidic epitope from an EBV antigen, optionally presented by an MHC) and this can trigger expansion of T cells specific for EBV. In another example, a virus-specific T cell for BK virus in a sample of PBMCs obtained from a suitable donor a virus-specific T cell for adenovirus in the sample of PBMCs can respectively recognize and bind to a BK virus antigen and an adenovirus antigen (e.g., a peptidic epitope from a BK virus antigen and an adenovirus antigen, respectively, optionally presented by an MHC) and this can trigger expansion of T cells specific for a BK virus and T cells specific for an adenovirus.

As used herein, the term “cell therapy product” refers to a cell line, e.g., as described herein, expanded and/or manufactured outside of a subject. For example, the term “cell therapy product” encompasses a cell line produced in a culture. The cell line may comprise or consist essentially of effector cells. The cell line may comprise or consist essentially of NK cells. The cell line may comprise or consist essentially of T cells. For example, the term “cell therapy product” encompasses an antigen specific T cell line produced in a culture. Such antigen specific T cell lines include in some instances expanded populations of memory T cells, expanded populations of T cells produced by stimulating naïve T cells, and expanded populations of engineered T cells (e.g., CAR-T cells and T cells expressing exogenous proteins such as chimeric or recombinant T cell receptors, co-stimulatory receptors, and the like). In particular, the term “cell therapy product” in some embodiments includes a virus specific T cell line or a tumor specific T cell line (e.g., TAA-specific T cell line). The cell line may be monoclonal or oligoclonal. In particular embodiments, the cell line is polyclonal. Such polyclonal cells lines comprise, in some embodiments, a plurality of expanded populations of cells (e.g., antigen specific T cells) with divergent antigen specificity. For example, one non-limiting example of a cell line encompassed by the term “cell therapy product” comprises a polyclonal population of virus specific T cells comprising a plurality of expanded clonal populations of T cells, at least two of which respectively have specificity for different viral antigens. Such antigen-specific T cells suitable for use in the compositions and methods of the present disclosure, including polyclonal virus specific T cells, can be made according to any method known in the art including at least any method disclosed in WO2011028531, WO2013119947, WO2017049291, WO2013/008147, PCT/US2020/046389, and PCT/US2020/024726, each of which is incorporated herein by reference in its entirety.

The term “donor minibank” as used herein refers to a plurality of cell therapy products (e.g., antigen-specific T cell lines) derived from different donors such that the cell therapy products within the donor minibank collectively provide a defined percentage of patients (e.g., >70%, >75%, >80%, >85, >90%, or >95%) in a target patient population with at least one well-matched cell therapy product (e.g., an antigen-specific T cell line).

For example, in certain embodiments, the donor minibanks described herein include at least one well-matched cell therapy product (e.g., antigen-specific T cell line) for at least 95% of a target patient population (such as, e.g., allogenic hematopoietic stem cell transplantation recipients or immunocompromised subjects). The term “donor bank” as used herein refers to a plurality of donor minibanks. In various embodiments, it is beneficial to create several non-redundant minibanks for inclusion in a “donor bank” to ensure the availability of two or more well-matched cell therapy products for each prospective patient. Cell banks may be cryopreserved. Cryopreservation methods are known in the art and may include, e.g., storage of the cell therapy products (e.g., antigen-specific T cell lines) at −70° C., e.g., in vapor-phase liquid nitrogen in a controlled-access area. Separate aliquots of cell therapy products may be prepared and stored in containers (e.g., vials) in multiple, validated, liquid nitrogen dewars. Containers (e.g., vials) may be labeled with unique identification numbers enabling retrieval.

As used herein, the terms “patient” or “subject” are used interchangeably to refer to any mammal, including humans, domestic and farm animals, and zoo, sports, and pet animals, such as dogs, horses, cats, cattle, sheep, pigs, goats, rats, guinea pigs, or non-human primates, such as a monkeys, chimpanzees, baboons or rhesus. One preferred mammal is a human, including adults, children, and the elderly.

As used herein, the term “potential donor” refers to an individual (e.g., a healthy individual) with seropositivity for the antigen or antigens that will be targeted by the cell therapy products (e.g., antigen specific T cells) disclosed herein. In some embodiments, all potential donors eligible for inclusion in the donor pools are prescreened and/or deemed seropositive for the target antigen(s).

The term “target patient population” is used in some embodiments to describe a plurality of patients (or “subjects” interchangeably) in need of a cell therapy product described herein (e.g., an antigen specific T cell product). In some embodiments, this term encompasses the entire worldwide allogeneic HSCT population. In some embodiments, this term encompasses the entire US allogeneic HSCT population. In some embodiments, this term encompasses all patients included in the National Marrow Donor Program (NMDP) database, available at the worldwide web address bioinformatics.bethematchclinical.org. In some embodiments, this term encompasses all patients included in the European Society for Blood and Marrow Transplantation (EBMT) database, available at the worldwide web address: ebmt.org/ebmt-patient-registry. In some embodiments, this term encompasses the entire worldwide allogeneic HSCT population of children ages ≤16 years. In some embodiments, this term encompasses the entire US allogeneic HSCT population of children ages ≤16 years. In some embodiments, this term encompasses the entire worldwide allogeneic HSCT population of children ages ≤5 years. In some embodiments, this term encompasses the entire US allogeneic HSCT population of children ages ≤5 years. In some embodiments, this term encompasses the entire worldwide allogeneic HSCT population of individuals ages ≥65. In some embodiments, this term encompasses the entire US allogeneic HSCT population of individuals ages ≥65.

The terms “treat”, “treating”, “treatment” and the like, as used herein, unless otherwise indicated, refers to reversing, alleviating, inhibiting the process of, or preventing the disease, disorder or condition to which such term applies, or one or more symptoms of such disease, disorder or condition and includes the administration of any of the compositions, pharmaceutical compositions, or dosage forms described herein, to prevent the onset of the symptoms or the complications, or alleviating the symptoms or the complications, or eliminating the disease, condition, or disorder. In some instances, treatment is curative or ameliorating.

Reference herein to the term “third party” in some embodiments means a subject (e.g., a patient) that is not the same as a donor. So, for example, reference to treating a subject with a “third party antigen-specific T cell product” (e.g., a third party VST product) means that the product is derived from donor tissue (e.g., PBMCs isolated from the donor's blood) and the subject (e.g., patient) is not the same subject as the donor. In various embodiments, an allogeneic cell therapy (e.g., an allogeneic antigen-specific T cell therapy) is a “third party” cell therapy.

The term “prevent” or “preventing” with regard to a subject refers to keeping a disease or disorder from afflicting the subject. Preventing includes prophylactic treatment. For instance, preventing can include administering to the subject a composition disclosed herein before a subject is afflicted with a disease and the administration will keep the subject from being afflicted with the disease. For example, preventing includes methods for preventing or controlling a viral infection or the reactivation of a latent virus via prophylactic administration of a universal antigen-specific T cell therapy product provided herein, e.g., in the context of an allogeneic T cell therapy setting.

The terms “administering”, “administer”, “administration” and the like, as used herein, refer to any mode of transferring, delivering, introducing, or transporting a therapeutic agent to a subject in need of treatment with such an agent. Such modes include, but are not limited to, intraocular, oral, topical, intravenous, intraperitoneal, intramuscular, intradermal, intranasal, and subcutaneous administration.

The term “UVST” means a universal VST as provided herein.

The terms universal antigen specific T cell composition, universal cell therapy product, universal antigen-specific cell therapy product, and the like are used interchangeably herein and refer to a cell therapy composition that comprises two or more antigen-specific T cell lines comprising populations of antigen-specific T cells as described herein, wherein said antigen-specific T cell lines are derived from donor material (e.g., MNCs or PBMCs) originating from at least two separate donors. The universal antigen-specific T cell therapy products and/or plurality of antigen-specific T cell lines may be in the form of a composition comprising each antigen-specific T cell line making up the product (i.e., two or more antigen-specific T cell lines), or may be in the form of a plurality of compositions of individual antigen-specific T cell lines for administration in a single dosing session. In embodiments, the universal antigen-specific T cell therapy product comprises a plurality of individual antigen-specific T cell lines generated from a suitable donor population. A suitable donor population may comprise a plurality of different donors, wherein the HLA type of each donor differs from at least one of the other donors on at least one HLA allele, as further described herein. In some embodiments, the universal antigen-specific T cell therapy product (e.g., a UVST) comprises a plurality of cell lines present in a donor minibank described herein or a donor bank described herein. In particular embodiments, a universal antigen-specific T cell therapy product (e.g. a UVST) comprises some or all of the cell lines present in a donor minibank described herein or a donor bank described herein.

In various embodiments, the term “well-matched” is used herein in reference to a given patient and a given cell therapy product (e.g., an antigen specific T cell line) to describe when the patient and the cell therapy product shares (i.e., is matched on) a pre-set threshold number of HLA alleles. For example, in embodiments, a cell therapy product is well matched to a patient if the patient and the cell therapy product are matched on at least two HLA alleles.

Other objects, feature and advantages of the present invention will become apparent from the following detailed description. It should be understood, however, that the detailed description and specific examples, while indicating specific embodiments of the invention, are given by way of illustration only, since various changes and modifications within the spirit and scope of the invention will become apparent to those skilled in the art from this detailed description.

The following discussion is directed to various embodiments of the invention. The term “invention” is not intended to refer to any particular embodiment or otherwise limit the scope of the disclosure. Although one or more of these embodiments may be preferred, the embodiments disclosed should not be interpreted, or otherwise used, as limiting the scope of the disclosure, including the claims. In addition, one skilled in the art will understand that the following description has broad application, and the discussion of any embodiment is meant only to be exemplary of that embodiment, and not intended to intimate that the scope of the disclosure, including the claims, is limited to that embodiment.

### Overview

The present disclosure provides universal antigen specific T cell compositions and products (e.g., universal VST compositions and universal VST products), and methods of making and using the same. The universal antigen-specific T cell compositions and products comprise populations of antigen-specific T cells derived from a plurality of different donors. In the plurality of different donors, the HLA type of each donor differs from at least one of the other donors on at least one HLA allele. In an aspect, the universal antigen-specific T cell compositions and products include T cells from a sufficient diversity of donors having diversity of HLA alleles such that the compositions and products achieve a high degree of matching across the entire patient population. In various embodiments, the plurality of different donors have sufficient diversity of HLA alleles (with respect to one another) such that the compositions and products match a large percentage of patients across an entire patient population on at least one HLA allele (e.g., 95% or more of a given patient population); for example, in particular aspects such composition and products match 95% or more of patients across an entire patient population on at least two HLA alleles. Thus, in aspects, a plurality of different HLA alleles are represented in the universal antigen-specific T cell composition such that the compositions and products can be universally administered to recipients without the need for HLA typing. This is in contrast traditional antigen specific T cell compositions and products, which are only administered to well-matched patents and, thus, require prior HLA typing of the patient.

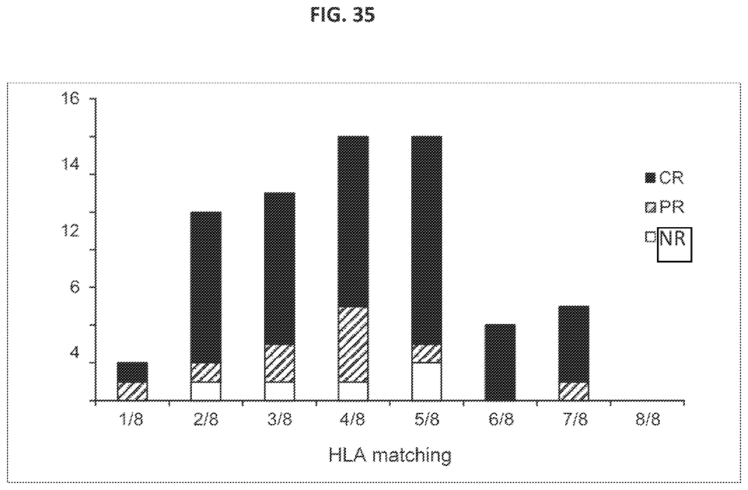

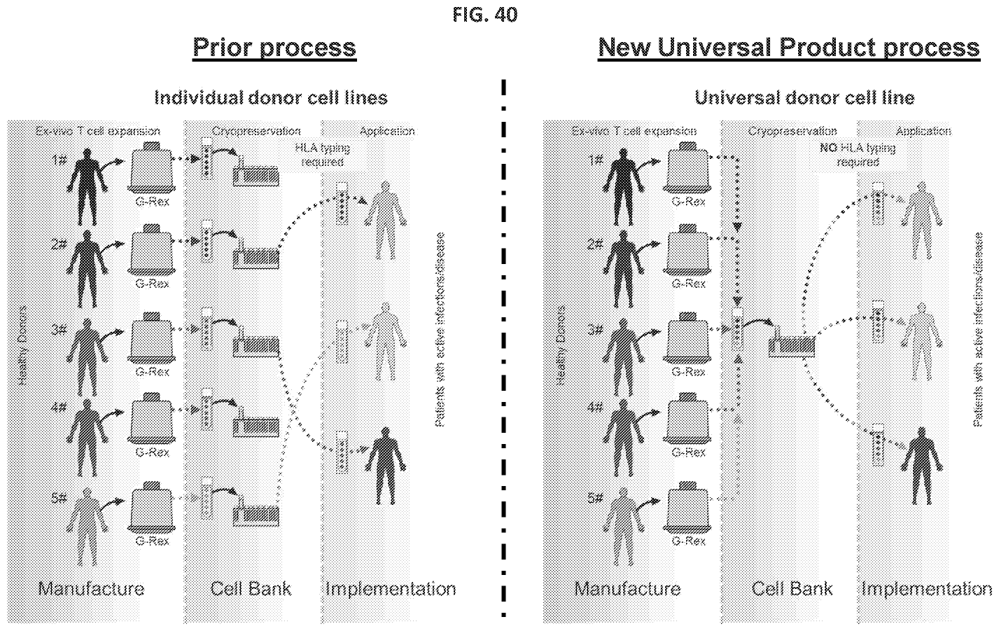

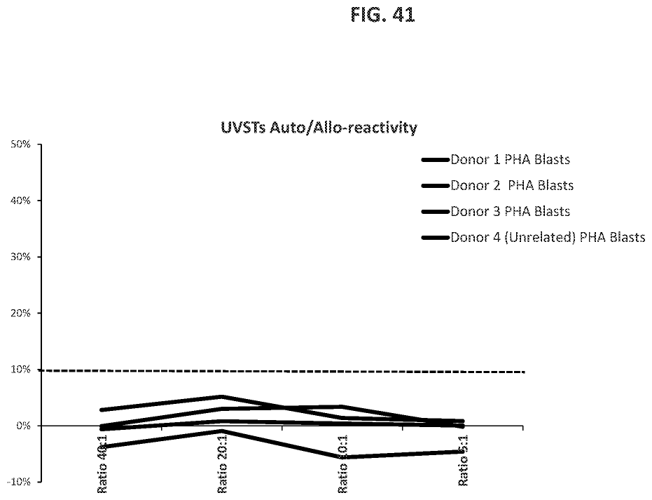

In aspects, the present disclosure is based on several surprising clinical and pre-clinical observations. First, partially-matched VST compositions are efficacious in patients, and in some instances complete or partial responses were observed even when patients were treated with products with as low as one matching allele (FIG. 35). Thus, these preliminary data support that a low-matched product provides therapeutic benefit. Second, administration to a given patient of two or more VST cell lines generated from donors with divergent HLA types results in treatment responses (Table 7), with no or little acute graft versus host disease (aGVHD) by week 6 or chronic GVHD (cGVHD) within 1 year of treatment (Table 8). Thus, these preliminary data support that treatment with multiple VST cell products with divergent HLA profiles may be safe and does not appear to reduce the therapeutic potential of the products. Third, in vitro data presented herein demonstrates that individual VST cell lines maintain potency post-pooling into a universal VST cell product (Tables 11 and 13) and lack auto-reactivity or allo-reactivity (FIGS. 40-41). Thus, taken together these data support that the universal antigen specific T cell compositions and products disclosed herein are highly advantageous over other T cell products; for example, the universal antigen-specific T cell composition or product may be administered to a patient in need thereof without the need to HLA type the patient (though prior HLA typing does not preclude administration), and without the need to select and/or generate an HLA-matched antigen-specific T cell line for administration to the patient or the need to generate an autologous antigen-specific T cell line for administration to the patient. Thus, the universal antigen specific T cell compositions and products provided herein, and the methods of use thereof, allow for rapid treatment of patients with an off-the-shelf product that can be administered to any patient. This may be particularly advantageous for treatment of viral infections with off-the-shelf virus-specific T cells in a pandemic setting (such as the SARS-CoV2 pandemic), as the treatment may be administered quickly to a patient without waiting for HLA typing that might delay access to treatment by a day or more. Thus, in embodiments, the universal antigen-specific T cell compositions and products provided herein may provide superior efficacy compared to traditional antigen-specific T cell compositions, including greater speed of response of the T cells, superior speed and efficacy with respect to disease improvement, and/or improved durability of treatment benefit.

In embodiments, the universal antigen-specific T cell compositions and products provided herein may be superior to other T cell products in that they provide better coverage of HLA alleles for a particular patient compared to coverage obtainable by employing traditional HLA matching. For example, the universal antigen-specific T cell product may include a plurality of antigen specific T cell lines (e.g., VST cell lines) from a plurality of donors that are at least partially matched or well matched to a recipient, and in a traditional therapeutic paradigm, only one of these well-matched T cell lines would be administered to the patient and able to effect treatment. However, with the universal antigen-specific T cell compositions and products provided herein, the product will include not only the most well-matched antigen-specific T cell product available, but will also likely include one or more additional T cell lines from multiple different donors that may be less-optimally matched to the recipient, but that are nevertheless likely to be therapeutically active based on our results provided here. That is, the universal antigen-specific T cell compositions provided herein are likely to contain more than one product within the composition that is partially HLA matched with the patient; and the different partially HLA matched products within the universal antigen-specific T cell composition may match the patient at different alleles. Thus, the universal antigen-specific T cell compositions and products disclosed herein will likely provide for broader antigen-specific activity than a single T cell product. Thus, without being limited by conjecture, an additive or synergistic response may be observed. Further, in embodiments, the compositions and methods provided herein are advantageous in that this superior coverage is achieved without the need for HLA typing of the recipient of the cell product. In embodiments, the universal antigen-specific T cell products provided herein are matched with a recipient on every allele.

In embodiments, the universal antigen-specific T cell compositions provided herein comprise antigen-specific T cell lines from a plurality of donors, pooled together into a single composition. In embodiments, patients are administered the pooled composition. In embodiments, the universal antigen-specific T cell compositions provided herein comprise individual antigen-specific T cell lines each from an individual donor, wherein the compositions are administered to a patient in a single dosing session. Thus, the universal antigen-specific T cell compositions may be administered as a pooled product with individual cell lines mixed together prior to administration. In embodiments, the cell lines may be pooled together prior to freezing the composition, wherein the pooled composition is thawed prior to administration to a patient; or may be pooled together and administered after individual cell lines are cryopreserved (i.e., cooled to very low temperature, typically about −80° C.) and then thawed. Alternatively, in embodiments, the universal antigen-specific T cell compositions may be pooled together and administered as a pooled product that has not undergone a freezing or thawing step. In embodiments, the antigen-specific T cell compositions may be administered to a patient as individual administrations of individual T cell lines, simultaneously or sequentially in a single dosing session. A “dosing session” as used herein refers to a session or visit with the medical professional administering the composition or compositions. In embodiments, a single dosing session may encompass several hours or days. In embodiments, compositions administered in a single dosing session are administered within minutes of one another, or within 1, 2, 3, 4, 5, 6, 12, or 18 hours of one another, or within about 1, 2, 3, 4, 5, 6, or 7 days. For example, in embodiments, dosing sessions involve the administration of two or more individual cell line compositions, wherein the patient does not leave the facility or location where the compositions are administered between doses and/or the patient does not undergo testing or assessments, such as assessments of efficacy or longevity of the cells in vivo, other than safety monitoring between doses (in case such monitoring is instituted or required). In some dosing sessions, the individual cell lines are pooled together at the time that the compositions are administered, e.g., by mixing individual vials prior to drawing the mixed composition up onto a syringe for administration. In some dosing sessions, the individual cell lines are pooled together in the syringe, e.g., cells are drawn into the syringe from two or more vials of individual cell line compositions. In some dosing sessions, the individual cell lines are administered separately to the patient in the single dosing session.

In embodiments, the cell therapy products provided herein comprise two or more universal antigen-specific T cell products pooled together. In embodiments, the cell therapy products provided herein comprise a universal antigen-specific T cell product to which one or more additional individual antigen-specific T cell lines is added. In embodiments, the present disclosure provides methods for personalized administration of T cell therapy products wherein a patient is administered a universal antigen-specific T cell product in combination with an additional universal antigen-specific T cell product and/or in combination with one or more additional individual antigen-specific T cell lines, so that a patient receives a universal antigen-specific T cell product that is highly personalized to the patient's needs and/or genetic profile. For example, in embodiments, the present disclosure provides methods wherein a patient's HLA type is known and a highly personalized antigen-specific T cell product that covers all or most of the patient's HLA alleles is prepared by pooling together two or more universal antigen-specific T cell products, two or more individual antigen-specific T cell lines, and/or one or more universal antigen-specific T cell product with one or more individual antigen-specific T cell line. In embodiments, the present disclosure provides methods for treating a patient with a highly personalized antigen-specific T cell product comprising administering two or more individual antigen-specific T cell lines simultaneously or sequentially to a patient in a single dosing session.

In aspects, the universal antigen-specific T cell compositions comprise pathogen-specific T cells and/or tumor specific (e.g., tumor antigen, or TAA) T cells. In particular aspects, the universal antigen-specific T cell compositions comprise virus-specific T cells and/or tumor specific (e.g., tumor antigen, or TAA) T cells. In embodiments, the universal antigen specific T cell therapies provided herein are universal virus specific T cell (UVST) compositions.

In embodiments, the cell lines making up the universal antigen-specific T cell compositions provided herein are monoclonal, oligoclonal, and/or polyclonal. In embodiments, each of the cell lines in the universal antigen-specific T cell composition is a polyclonal cell line. In embodiments, the universal antigen-specific T cell composition comprises one or more monoclonal cell lines in combination with one or more oligoclonal cell lines; one or more monoclonal cell lines in combination with one or more polyclonal cell lines; or one or more oligoclonal cell lines in combination with one or more polyclonal cell lines.

### Donors and HLA Types

As provided herein, the universal antigen-specific T cell compositions provided herein comprise a population of antigen-specific T cells comprising a plurality of antigen-specific T cell lines derived from a plurality of different donors, wherein the HLA type of each donor differs from at least one of the other donors on at least one HLA allele. In embodiments, the HLA type of each donor differs from at least one of the other donors on 1, 2, 3, 4, 5, 6, 7 or 8 HLA alleles. In embodiments, the universal antigen-specific T cell compositions provided herein are generated by pooling cells in a donor minibank. In embodiments, the plurality of different donors are in a donor population of 2, 3, 4, 5, 6, 7, 8, 9, 10, 11, 12, 13, 14, 15, or more donors. In embodiments, the plurality of different donors are in a donor population of 15 or fewer donors, 10 or fewer donors, or 5 or fewer donors. In embodiments, the HLA type of each donor (e.g., in a donor population of 15 or fewer donors, 10 or fewer donors, or 5 or fewer donors) differs from at least 2, at least 3, at least 4, at least 5, at least 6, at least 7, at least 8, at least 9, at least 10, or all of the other donors on at least one HLA allele. In embodiments, the HLA type of each donor differs from at least 2, at least 3, at least 4, at least 5, at least 6, at least 7, at least 8, at least 9, at least 10, or all of the other donors on at least two HLA alleles. In embodiments, the HLA type of each donor differs from at least 2, at least 3, at least 4, at least 5, at least 6, at least 7, at least 8, at least 9, at least 10, or all of the other donors on at least three HLA alleles. In embodiments, the HLA type of each donor differs from at least 2, at least 3, at least 4, at least 5, at least 6, at least 7, at least 8, at least 9, at least 10, or all of the other donors on at least four HLA alleles. In embodiments, the HLA type of each donor differs from at least 2, at least 3, at least 4, at least 5, at least 6, at least 7, at least 8, at least 9, at least 10, or all of the other donors on at least five HLA alleles. In embodiments, the HLA type of each donor differs from at least 2, at least 3, at least 4, at least 5, at least 6, at least 7, at least 8, at least 9, at least 10, or all of the other donors on 6 HLA alleles.

In embodiments, the HLA type of each donor (e.g., in a donor population of 15 or fewer donors, 10 or fewer donors, or 5 or fewer donors) differs from at least one other donor on one or more class I HLA allele. In embodiments, the HLA type of each donor differs from at least 2, 3, 4, 5, 6, 7, 8, 9, 10, or all of the other donors on one or more class I HLA allele. In embodiments, the HLA type of each donor differs from at least one other donor on one or more HLA-A, HLA-B, and/or HLA-C allele. In embodiments, the HLA type of each donor differs from at least 2, 3, 4, 5, 6, 7, 8, 9, 10, or all of the other donors on one or more HLA-A, HLA-B, and/or HLA-C allele. In embodiments, the HLA type of each donor differs from at least one other donor on at least one HLA-A and at least one HLA-B allele. In embodiments, the HLA type of each donor differs from at least 2, 3, 4, 5, 6, 7, 8, 9, 10, or all of the other donors on at least one HLA-A and at least one HLA-B allele. In embodiments, the HLA type of each donor differs from at least one other donor on at least one HLA-A and at least one HLA-B allele and at least one HLA-C allele. In embodiments, the HLA type of each donor differs from at least 2, 3, 4, 5, 6, 7, 8, 9, 10, or all of the other donors on at least one HLA-A and at least one HLA-B allele and at least one HLA-C allele.

In embodiments, the HLA type of each donor (e.g., in a donor population of 15 or fewer donors, 10 or fewer donors, or 5 or fewer donors) differs from at least one other donor on one or more class II HLA allele In embodiments, the HLA type of each donor differs from at least 2, 3, 4, 5, 6, 7, 8, 9, 10, or all of the other donors on at least one or more class II HLA allele. In embodiments, the HLA type of each donor differs from at least one other donor on one or more DP, DQ, and/or DR allele. In embodiments, the HLA type of each donor differs from at least 2, 3, 4, 5, 6, 7, 8, 9, 10, or all of the other donors on one or more DP, DQ, and/or DR allele. In embodiments, the HLA type of each donor differs from at least one other donor on one or more of HLA-DQA1, HLA-DQB1, HLA-DRA, and/or HLA-DRB. In embodiments, the HLA type of each donor differs from at least one other donor on one or more of HLA-DQA1, HLA-DQB1, HLA-DRA, and HLA-DRB. In embodiments, the HLA type of each donor differs from at least one other donor on one or more of HLA-DPA1, HLA-DPB1, HLA-DQA1, HLA-DQB1, HLA-DRA, and/or HLA-DRB.

In embodiments, the HLA type of each donor (e.g., in a donor population of 15 or fewer donors, 10 or fewer donors, or 5 or fewer donors) differs from at least one other donor on at least one class I HLA allele and at least one class II HLA allele. In embodiments, the HLA type of each donor differs from one or more of the other donors one at least two class I HLA allele and at least two class II HLA alleles. In embodiments, the donors have at least 2 different HLA-A alleles, at least 2 different HLA-B alleles, at least 2 different DRB1 alleles, and/or at least 2 different DQB1 alleles.

### Cells Expressing Exogenous (Transgenic) Molecules

In embodiments, the universal antigen-specific T cell compositions provided herein comprise T cells that express an exogenous molecule. In embodiments, such T cells are UVSTs as described herein. Expression of exogenous molecules may be achieved by any of a number of appropriate means which are known in the art, including electroporation, nucleofection, transfection employing liposomes or calcium chloride, rubidium chloride, calcium phosphate, DEAE-dextran, or other substances; microprojectile bombardment; lipofection; transposons or transposases; and infection (e.g., where the vector is an infectious agent such as a virus). In embodiments, the exogenous molecule is encoded by DNA or RNA. In embodiments, the DNA or RNA may be modified DNA or modified RNA. In embodiments, the exogenous molecule is encoded by an mRNA or a polynucleotide which may exist in an expression cassette or expression vector (e.g., plasmids, viral vectors, including viruses (e.g., lentiviruses), adenoviruses, adeno-associated viruses, or cosmids), and the T cells are transfected with the mRNA or vector to achieve expression of the exogenous molecule. In embodiments, the universal antigen-specific T cell compositions provided herein comprise T cells that have been transduced with a retrovirus or lentivirus vector containing a transgene encoding an exogenous molecule, wherein the exogenous molecule is a CAR, a transgenic TCR, an NK cell receptor, or a therapeutic agent. In embodiments, the exogenous molecule is a CAR or a TCR. In embodiments, the exogenous molecule is a CAR comprising an antigen binding domain specific for a cancer antigen.

In embodiments, the universal antigen-specific T cell compositions provided herein are used as carriers for exogenous molecules, wherein the composition provides a safe delivery vehicle for T cell therapies that advantageously contain a mixture of cell lines from a plurality of different donors. In embodiments, such universal antigen-specific T cell compositions for use as carriers for exogenous molecules are UVSTs. The antigen-specific T cell compositions contain a mixture of cell lines from a plurality of different donors such that some cells may be mismatched while some are partially matched to a recipient patient. Such mixture of different cell lines provides a composition capable of persisting in the recipient without the need for further modification of the cells to prevent rapid rejection upon administration to the recipient. In embodiments, the universal antigen-specific T cell compositions which have been modified to express an exogenous molecule comprise modified T cells that lack alloreactivity against host cells and are capable of persisting in the patient. In embodiments, the compositions comprise modified T cells that persist in the patient for a sufficient time period to perform a desired function, e.g., targeting and eradicating cancer cells.

In embodiments, the universal antigen-specific T cell compositions provided herein that express an exogenous molecule perform dual functions. For example, in embodiments, a first function is the effector activity in connection with the exogenous molecule (e.g., anti-tumor cell activity of a T cell expressing a CAR specific for a tumor antigen on a tumor cell); and a second function is the effector activity in connection with the specificity of the native T cell receptor expressed by the T cells themselves (e.g., for a plurality of tumor antigens and/or a plurality of viral antigens). For example, in embodiments, the present disclosure provides universal antigen-specific T cell compositions wherein the T cells have been modified to express a CAR and have anti-tumor activity via the CAR as well as anti-viral activity to prevent or reduce viral infection or to prevent or treat viral reactivations or lytic virus infections in the patient receiving the therapy.

In other embodiments, the universal antigen-specific T cell compositions provided herein that express an exogenous molecule do not perform a function in connection with the specific antigen specificity of the T cells, but are suitable as safe carriers for exogenous molecules as provided above. In such embodiments, the universal antigen-specific T cell compositions may traffic to sites of inflammation where the cells will perform an effector function.

In embodiments, T cells of the universal antigen-specific T cell compositions provided herein have been engineered to express a CAR. The term “chimeric antigen receptor” (“CAR”), as used herein, refers to a recombinant polypeptide construct comprising at least an extracellular antigen binding domain, a transmembrane domain and a cytoplasmic domain. In embodiments, the cytoplasmic domain comprises an intracellular signaling domain. In embodiments, the intracellular signaling domain comprises a functional signaling domain derived from a stimulatory molecule (e.g., a molecule that provides the primary cytoplasmic signaling sequence(s) to regulate or induce primary activation of the TCR complex in a stimulatory manner). In embodiments, the intracellular signaling domain further comprises a costimulatory molecule. For example, in embodiments, the intracellular signaling domain comprises a primary signaling domain (e.g., a primary signaling domain comprising an immmunoreceptor tyrosine-based activation motif (iTAM), such as CD3-zeta), and optionally one or more functional signaling domains derived from at least one costimulatory molecule. CD27, ICOS, and/or CD28). Exemplary primary signaling domains include TCRζ, FcRγ, FcRβ, FcRε, CD3γ, CD3ζ, CD3ε, CD5, CD22, CD79a, CD79b and CD66d. Exemplary costimulatory molecules include CD27, CD28, CD8, 4-1 BB (CD137), OX40, CD30, CD40, PD-1, ICOS, lymphocyte function-associated antigen-1 (LFA-1), CD2, CD7, LIGHT, NKG2C, an MHC class I molecule, BTLA, a TLR, and B7-H3. Exemplary co-stimulatory ligands which bind to a cognate co-stimulatory signal molecule on a T cell include CD7, B7-1 (CD80), B7-2 (CD86), PD-L1, PD-L2, 4-1BBL, OX40L, inducible costimulatory ligand (ICOS-L), intercellular adhesion molecule (ICAM, CD30L, CD40, CD70, CD83, HLA-G, MICA, M1CB, HVEM, lymphotoxin β receptor, 3/TR6, ILT3, ILT4, an agonist or antibody that binds a TLR, and a ligand that specifically binds with B7-H3. A co-stimulatory ligand also encompasses, inter alia, an antibody that specifically binds with a co-stimulatory molecule present on a T cell, such as but not limited to, CD27, CD28, 4-1BB, OX40, CD30, CD40, PD-1, ICOS, lymphocyte function-associated antigen-1 (LFA-1), CD2, CD7, LTGHT, NKG2C, B7-H3, and a ligand that specifically binds with CD83.

In embodiments, T cells of the universal antigen-specific T cell compositions provided herein have been engineered to express an exogenous TCR (e.g., an αβ TCR or a γδ TCR). In embodiments, T cells of the universal antigen-specific T cell compositions provided herein have been engineered to express an NK cell receptor. Exemplary NKT or NK cell receptors include NKG2D, NKp30, NKp44, NKp46. KIR2DS1, KIR2DS2/3, KIR2DL4, KIR2DS4, KIR2DS5, KIR3DS1, and NKG2C. In embodiment, exogenous TCRs or NK cell receptors, like CARs, include the intracellular signaling domain that triggers effector function in the cell.

In embodiments, the T cells express one or more therapeutic agent or molecule, such as one or more pro-inflammatory cytokines and/or ligands, or one or more chemotherapeutic agents. For example, in embodiments, the T cells have been genetically modified to express one or more of IL-2, IL-6, IL-7, IL-12, IL-15, IL-15, IL-15/IL-15RA, IL-18, IL-21, TNFα, IFNγ, chimeric receptor, chimeric cytokine receptor, methotrexate, aminopterin, 6-mercaptopurine, 6-thioguanine, cytarabine, 5-fluorouracil decarbazine, mechlorethamine, thioepa chlorambucil, melphalan, carmustine (BSNU), mitomycin C, lomustine (CCNU), 1-methylnitrosourea, cyclothosphamide, mechlorethamine, busulfan, dibromomannitol, streptozotocin, mitomycin C, cis-dichlorodiamine platinum (II) (DDP), cisplatin, carboplatin, cisplatin and carboplatin (paraplatin); daunorubicin, doxorubicin (adriamycin), detorubicin, carminomycin, idarubicin, epirubicin, mitoxantrone and bisantrene; antibiotics include dactinomycin (actinomycin D), bleomycin, calicheamicin, mithramycin, anthramycin (AMC), vinca alkaloid, vincristine, vinblastine, paclitaxel (taxol), ricin, pseudomonas exotoxin, gemcitabine, cytochalasin B, gramicidin D, ethidium bromide, emetine, etoposide, tenoposide, colchicin, dihydroxy anthracin dione, 1-dehydrotestosterone, glucocorticoids, procaine, tetracaine, lidocaine, propranolol, puromycin, procarbazine, hydroxyurea, asparaginase, corticosteroids, or mytotane (O,P′-(DDD)).

In embodiments, the exogenous molecules are one or more small molecules kinase inhibitors, or inhibitors of elements of the tumor microenvironment. In embodiments, the exogenous molecules are one or more receptors that sequester inhibitor molecules at a tumor site.

In embodiments, the present disclosure provides methods for generating universal antigen-specific T cell compositions wherein one or more of the T cells in the composition expresses an exogenous molecule (e.g., a therapeutic agent, a CAR, an exogenous TCR, NKT or an NK cell receptor). In embodiments, individual T cell lines generated as described herein are engineered to express the exogenous molecule. In other embodiments, the individual T cell lines are pooled together as provided herein, and the pooled cell product is engineered to express the exogenous molecule. In embodiments, the individual T cell lines and/or the pooled cell product is tested to assess the percent expression of the exogenous molecule prior to using the cell lines and/or pooled cell product in a method of treatment provided herein.

### Donor Minibanks

Embodiments of the present disclosure include donor minibanks containing a plurality of cell therapy products (e.g., antigen-specific T cell lines) and donor banks made up of a plurality of such donor minibanks, as well as methods of making and using such donor minibanks, donor banks, and the cell therapy products (e.g., antigen specific T cell lines) contained therein (alone or in combination as universal cell therapy products) for use adoptive immunotherapy to treat diseases or disorders.

In embodiments, the universal antigen-specific T cell products provided herein are generated by pooling together some or all cell lines of a donor minibank. For example, in embodiments, the universal antigen-specific T cell products provided herein are generated by pooling all of the cell lines in a donor minibank together. In embodiments, pooling together all of the cell lines in a minibank results in a composition that provides >95% coverage for a patient population. In embodiments, the present disclosure provides pooling together a subset of cell lines in a minibank, and/or comprising pooling together individual antigen-specific T cell lines, wherein the pooled composition provides >75%, >80%, >85%, >90%, or >95% coverage of a patient population. In embodiments, the present disclosure provides compositions comprising cell lines of a donor minibank.

In particular embodiments, the present disclosure includes methods and computer implemented algorithms for identifying and selecting a suitably-diverse set of donors (in terms of their HLA typing) for use in constructing cell therapy products (e.g., pluralities of antigen-specific T cell lines and universal antigen-specific T cell products) contained in or generated from donor minibanks to ensure that each donor minibank contains at least one well-matched cell therapy product (e.g., an antigen-specific T cell line) for a desired percentage of a target population. As is discussed further herein, the percentage of the target population that will be well-matched to at least one cell therapy product (e.g., an antigen specific T cell line) in a given minibank is a parameter that can be predetermined when the minibank is being constructed, and based on the HLA types of the target population and the number of cell therapy products included in the donor minibank. In some instances, each donor minibank contains at least one well-matched cell therapy product (e.g., an antigen-specific T cell line) to at least 70%, at least 80%, at least 85%, at least 90%, at least 95%, at least 96%, at least 97%, at least 98%, at least 99%, at least 99.9% of prospective patients in a target population, inclusive of all ranges and subranges therebetween. Thus, in some embodiments, the methods disclosed herein allow construction of such donor minibanks with suitable diversity of donors (in terms of their HLA typing) to ensure that at least one cell therapy product (e.g., an antigen-specific T cell line) in the donor minibank will be matched on at least 2 HLA alleles with 95% or more of a given target population.

In particular embodiments, the donors utilized in making such cell therapy products (e.g., antigen-specific T cell lines) contained in such a donor minibank are carefully selected using a donor selection method disclosed herein to ensure sufficient HLA variety between the donors such that at least 95% of a target patient population is matched on two or more HLA alleles with at least one cell therapy product in the minibank (e.g., an antigen-specific T cell line). This disclosure is based in part on the surprising discovery that partially HLA-matched cellular therapies, such as antigen-specific T cell lines (e.g., VST cell lines) are both safe and efficacious in third parties. Indeed, as is shown in Examples 1-3, our clinical trials have demonstrated that third party VSTs are safe and efficacious when administered to a subject that is matched on as little as one HLA allele (see, e.g., FIG. 35-37).

The present disclosure includes donor minibanks (and donor banks comprising a plurality of such donor minibanks), which donor minibanks include such cell therapy products derived from the blood samples collected from such suitable third party blood donors identified via the donor selection methods disclosed herein, as well as methods of making, administering, and using such cell therapy products (including, for example antigen-specific T cell line products, e.g., VSTs products), for treating or preventing diseases or disorders. Thus, in various embodiments, such donor minibanks include a plurality of cell therapy products (e.g., antigen-specific T cell lines) derived from samples (e.g., mononuclear cells such as PBMCs) obtained from the donors carefully selected using a donor selection method disclosed herein, and the cell therapy products therefor comprise sufficient HLA variety between one another such that at least 95% of the target patient population is matched on two or more HLA alleles with at least one cell therapy product in the minibank (e.g., an antigen-specific T cell line).

In various embodiments, one or more of the cell therapy products included in the donor minibanks disclosed herein are administered to a well-matched subject in need of such a therapy based on a patient matching method disclosed herein. In some embodiments, a plurality of such cell therapy products included in the donor minibank are administered to a well-matched subject based on a patient matching method disclosed herein. In some embodiments, a plurality of such cell therapy products included in the donor minibank are administered to a subject irrespective of whether the subject's HLA type is known. For example, as is discussed further below, in some such embodiments, the subject may be administered each of the cellular therapy products included in a donor minibank, which minibank includes a plurality of cell therapy products (e.g., antigen-specific T cell lines) derived from samples (e.g., PBMCs) obtained from the donors carefully selected using a donor selection method disclosed herein, and which cell therapy products therefore comprise sufficient HLA variety between one another such that at least 95% of the target patient population is matched on two or more HLA alleles with at least one cell therapy product in the minibank (e.g., an antigen-specific T cell line). In this manner, the donor minibank serves as a universal cell therapy product that is compatible (i.e., well-matched) with >95% of the target patient population. The plurality of cell therapy products that are administered together to the subject may be administered sequentially or simultaneously. In some embodiments, the plurality of the cell therapy products are pooled together and administered to the subject as a single universal cell therapy product. Such a pool of the cell therapy products (e.g., antigen specific T cell lines) contained in a donor minibank may be stored in a cell bank (e.g., under cryopreservation) for later administration to a subject in need thereof.

In some embodiments, the donors utilized in constructing the donor minibanks disclosed herein are pre-screened for seropositivity and/or the donors are healthy. The present disclosure provides that these antigen-specific T cell lines are prospectively generated and then cryopreserved so that they are immediately available as an “off the shelf” product with demonstrable immune activity against the infecting virus or multiple viruses.

The present disclosure provides, in some embodiments, that polyclonal VSTs may be made without requiring the presence of live viruses or recombinant DNA technologies in the manufacturing process. In some embodiments, T cell populations are expanded and enriched for virus specificity with a consequent loss in alloreactive T cells. The present disclosure also provides that the cell therapy (e.g., VST) donor banks and donor minibanks may in some embodiments be designed to accommodate >95% of an allogeneic HSCT patient population (e.g., the US allogeneic HSCT patient population). In addition, the cell therapy (e.g., VST) donor banks and donor minibanks are sufficiently HLA-matched to mediate antiviral effects against virally infected cells. For example, sufficiently HLA-matched indicates that at least 2 alleles are matched. The present disclosure provides in some embodiments, cell therapy products, e.g., VSTs, that are only partially matched with a subject's stem cell donor, and as a result, such cell therapy products (e.g., VSTs) are expected to circulate only until a time that the stem cell donor's cells fully repopulate the recipient, at which point the cell therapy product (e.g., VSTs) will be rejected by the patient's reconstituted immune system.

In some embodiments, the VSTs circulate in the recipient for up to 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, inclusive of all ranges and subranges therebetween. In one embodiment, the VSTs circulate in the recipient for up to 12 weeks

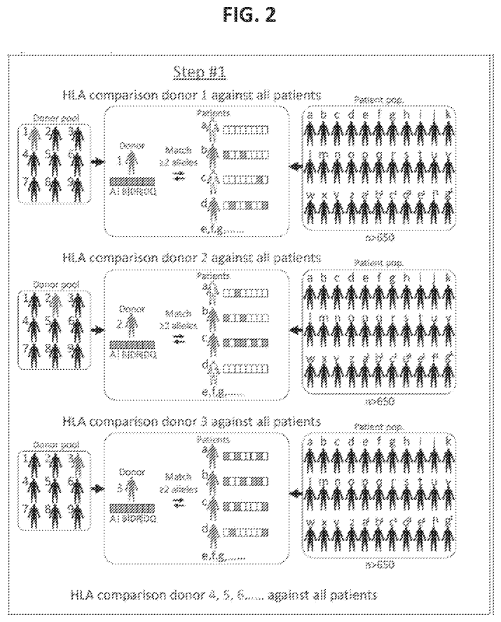

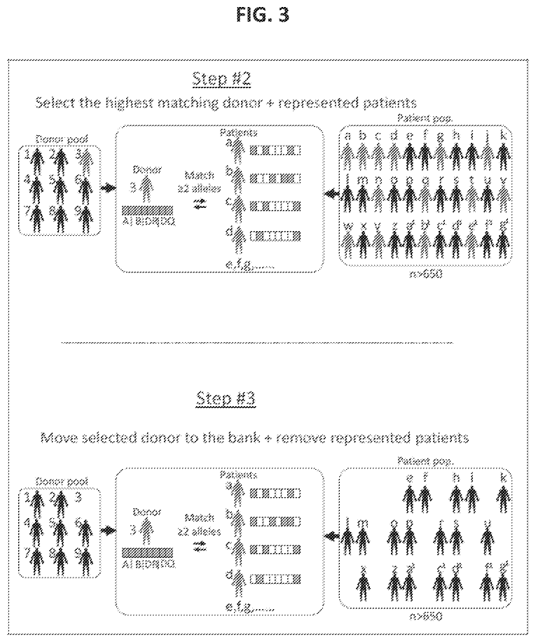

In some embodiments, the methods of identifying suitable donors for use in constructing a first donor minibank of antigen-specific T cell lines as described herein comprise step (a) comparing an HLA type of each of a first plurality of potential donors from a first donor pool with each of a first plurality of prospective patients from a first prospective patient population. In some embodiments, determining, based on the comparison in step (a) as described herein, a first greatest matched donor, defined as the donor from the first donor pool that has 2 or more HLA allele matches with the greatest number of patients in the first plurality of prospective patients (FIG. 2). In some embodiments, the donor who accommodates the majority of patients is (i) shortlisted for antigen-specific T cell line production, (ii) removed from the general donor pool, and (iii) all patients accommodated by this donor are removed from the patient population (FIG. 3). In Some embodiments, the first greatest matched donor is selected for the first donor minibank. In some embodiments, the methods as described herein comprise (d) removing from the first donor pool the first greatest matched donor thereby generating a second donor pool consisting of each of the first plurality of potential donors from the first donor pool except for the first greatest matched donor. In some embodiments, the methods as described herein comprise (e) removing from the first plurality of prospective patients each prospective patient that has 2 or more allele matches with the first greatest matched donor, thereby generating a second plurality of prospective patients consisting of each of the first plurality of prospective patients except for each prospective patient that has 2 or more allele matches with the first greatest matched donor.

In some embodiments, the first donor minibank comprises antigen-specific T cell lines derived from 10 or less, 9 or less, 8 or less, 7 or less, 6 or less, 5 or less, 4 or less, 3 or less, or 2 or less donors and comprises enough HLA variability to provide >95% of the first prospective patient population with one or more antigen-specific T cell line that is matched to the patient's HLA type on at least 2 HLA alleles. In some embodiments, the first donor minibank comprises antigen-specific T cell lines derived from 10 or less donors. In some embodiments, the first donor minibank comprises antigen-specific T cell lines derived from 5 or less donors.

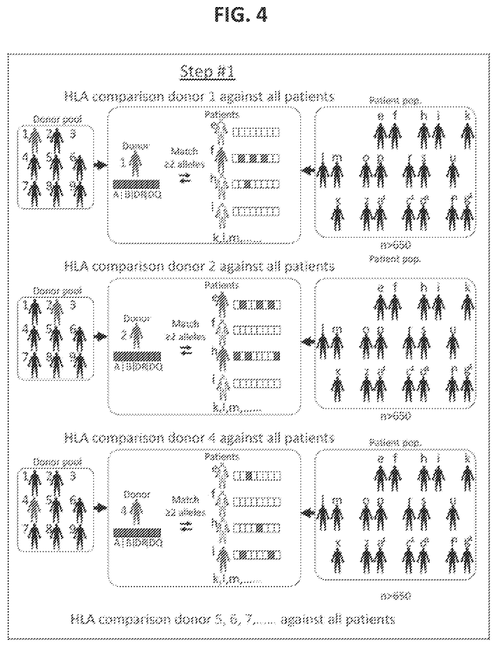

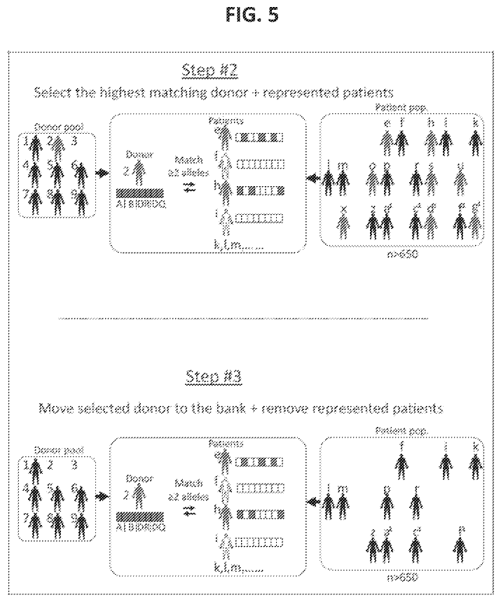

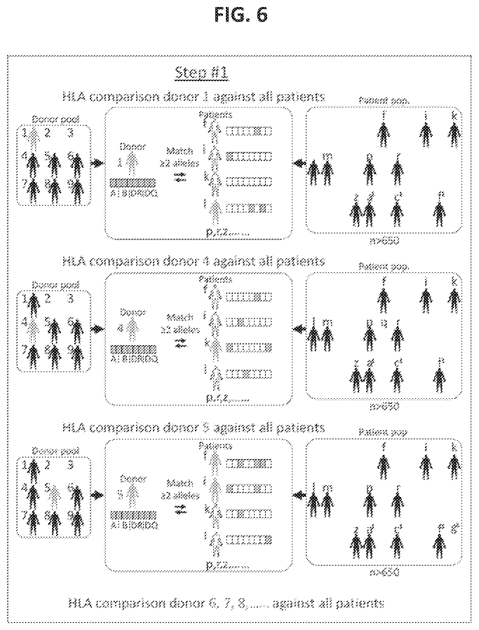

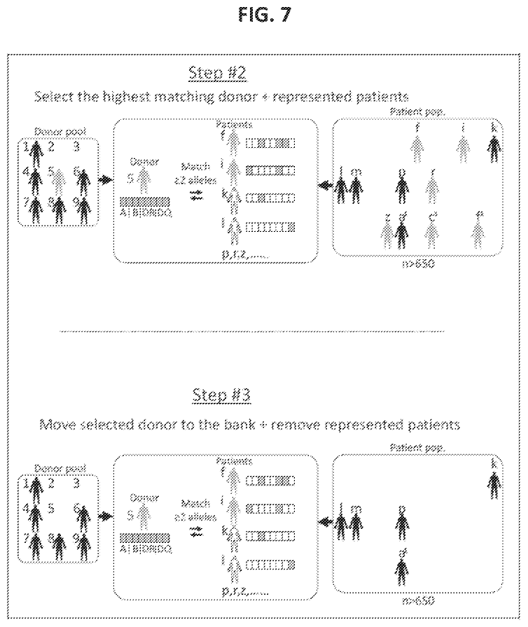

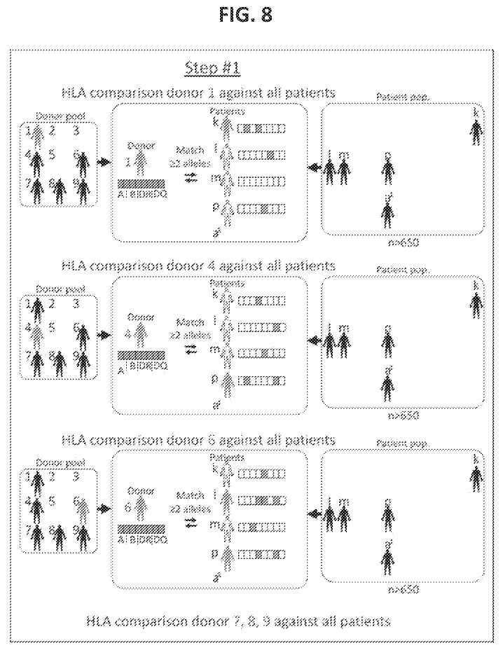

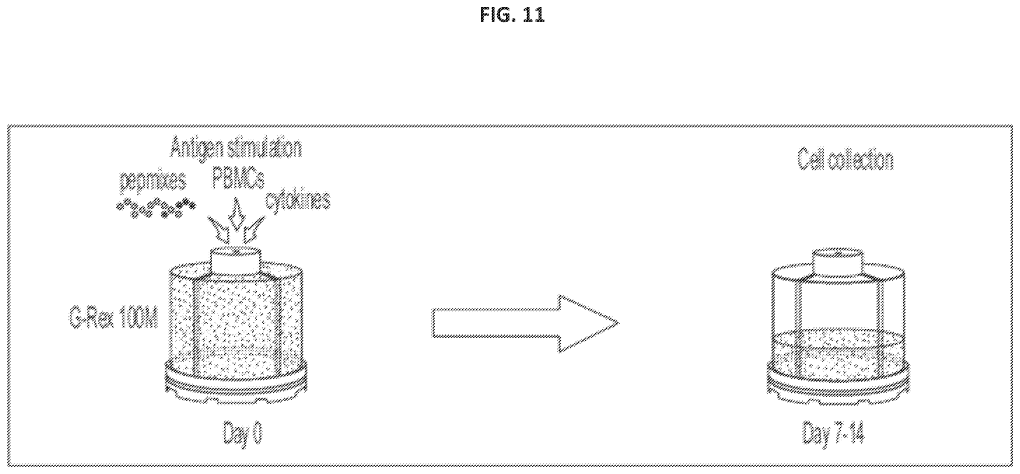

In some embodiments, as shown in FIG. 4-FIG. 10, the present methods comprise step (f) that repeats steps (a) through (e) as described herein at least one, at least two, at least three, at least four, at least five, at least six, at least seven, at least right, at least nine, at least ten times or more additional times with all donors and prospective patients that have not already been removed in accordance with steps (d) and (e). In some embodiments, steps (a) through (e) are repeated until a desired percentage of the first prospective patient population remains in the plurality of prospective patients or until no donors remain in the donor pool. In some embodiments, steps (a) through (e) as described herein are cycled in accordance with step (f) until 5% or less of the first prospective patient population remains in the plurality of prospective patients. As shown in FIG. 11, the first donor minibank is completed when the selected donors can represent at least 95%, at least 96%, at least 97%, at least 98%, at least 99%, at least 99.9% prospective patients, inclusive of all ranges and subranges therebetween.

In some embodiments, the first prospective patient population comprises at least 95, at least 97, at least 99, at least 100, at least 105, at least 110, at least 115, at least 120 patients. In some embodiments, the first prospective patient population comprises at least 100 patients.

In some embodiments, each time an additional greatest matched donor is selected in accordance with step (c) as described herein that additional greatest matched donor is removed from their respective donor pool in accordance with step (d). In some embodiments, each time a subsequent greatest matched donor is removed from their respective donor pool, each prospective patient that has 2 or more allele matches with that subsequent greatest matched donor is removed from their respective plurality of prospective patients in accordance with step (e).

In some embodiments, repeating steps (a) through (e) as described herein sequentially increase the number of selected greatest matched donors in the first donor minibank by 1 following each cycle of the method and thereby depleting the number of the plurality of prospective patients in the patient population following each cycle of the method in accordance with their HLA matching to the selected greatest matched donors. In some embodiments, the first donor minibank is completed when selected donor populations can cover at least 95% of the patients. In some embodiments, to ensure that each patient has multiple antigen-specific T cell line options, additional minibanks using the same strategy as described herein can be constructed.

In some embodiments, the 2 or more alleles comprise at least 2 HLA Class I alleles. In some embodiments, the 2 or more alleles comprise at least 2 HLA Class II alleles. In some embodiments, the 2 or more alleles comprise at least 1 HLA Class I allele and at least 1 HLA Class II allele.

In some embodiments, the first prospective patient population comprises the entire worldwide allogeneic HSCT population. In some embodiments, the first prospective patient population comprises the entire US allogeneic HSCT population. In some embodiments, the first prospective patient population comprises all patients included in the National Marrow Donor Program (NMDP) database, available at the worldwide web address bioinformatics.bethematchclinical.org. In some embodiments, the first prospective patient population comprises all patients included in the European Society for Blood and Marrow Transplantation (EBMT) database, available at the worldwide web address: ebmt.org/ebmt-patient-registry. In Some embodiments, the entire US allogeneic HSCT population can be determined by using a surrogate, where the sample size of said surrogate is large enough and is also representative for the US allogenic HSCT population. By way of examples, the 666 allogenic HSCT recipients at Baylor College of Medicine (Houston, TX) would be a suitable surrogate of the entire US allogeneic HSCT population. In some embodiments, the entire worldwide allogeneic HSCT population can be determined by using a surrogate, where the sample size of said surrogate is large enough and is also representative for the worldwide allogenic HSCT population. In some embodiments, the entire worldwide allogeneic HSCT population comprises children ages ≤3, ≤4, ≤5, ≤6, ≤7, ≤8, ≤9, ≤10, ≤11, ≤12, ≤13, ≤14, ≤15, ≤16, ≤17 years. In some embodiments, the entire worldwide allogeneic HSCT population comprises children ages ≤5 years. In some embodiments, the entire worldwide allogeneic HSCT population comprises children ages ≤16 years. In some embodiments, the entire worldwide allogeneic HSCT population comprises individuals ages ≥65, ≥70, ≥75, ≥80, ≥85, ≥90 years. In some embodiments, the entire worldwide allogeneic HSCT population comprises individuals ages ≥65 years. In some embodiments, the entire US allogeneic HSCT population comprises children ages ≤3, ≤4, ≤5, ≤6, ≤7, ≤8, ≤9, ≤10, ≤11, ≤12, ≤13, ≤14, ≤15, ≤16, ≤17 years. In some embodiments, the entire US allogeneic HSCT population comprises children ages ≤5 years. In some embodiments, the entire US allogeneic HSCT population comprises children ages ≤16 years. In some embodiments, the entire US allogeneic HSCT population comprises individuals ages ≥65, ≥70, ≥75, ≥80, ≥85, ≥90 years. In some embodiments, the entire US allogeneic HSCT population comprises individuals ages ≥65 years.

In some embodiments, the donor bank can be made by constructing a first minibank of antigen-specific T cell lines as described herein. In some embodiments, making the donor bank comprises repeating all the steps of constructing the first minimank as described herein. In some embodiments, making the donor bank comprises one or more second rounds to construct one or more second minibanks.

Prior to starting each second round, a new donor pool is generated. In some embodiments, the new donor pool comprises the first donor pool, less any greatest matched donors removed in accordance with each prior cycle of step (d) of constructing the first donor minibank, from the first and any prior second rounds of the method. In some embodiments, the new donor pool comprises an entirely new population of potential donors not included in the first donor pool. For example, the new donor pool can comprise potential donors that are completely different than the first donor pool. In some embodiments, the new donor pool can comprise a combination of the first donor pool, less any greatest matched donors removed in accordance with each prior cycle of step (d) of constructing the first donor minibank, from the first and any prior second rounds of the method, and an entirely new population of potential donors not included in the first donor pool. By way of example, the new donor pool can comprise three of the donors from the first donor pool and 7 new donors who are not in the first donor pool.

In some embodiments, prior to starting each second round, the method of constructing a donor bank comprises reconstituting the first plurality of prospective patients from the first prospective patient population. In some embodiments, the reconstituting comprises returning all prospective patients that had been previously removed in accordance with each prior cycle of step (e) (i.e. removing from the first plurality of prospective patients each prospective patient that has 2 or more allele matches with the first greatest matched donor, thereby generating a second plurality of prospective patients consisting of each of the first plurality of prospective patients except for each prospective patient that has 2 or more allele matches with the first greatest matched donor) from the first and any prior second rounds of the method.

In some embodiments, methods of constructing a first donor minibank of antigen-specific T cell lines comprise isolating MNCs, or having MNCs, isolated, from blood obtained from each respective donor included in the donor minibank. The blood from each donor included in the donor bank can be harvested. In some embodiments, mononuclear cells (MNCs) in the harvested blood from each donor included in the donor bank are collected. MNCs and PBMCs are isolated by using the methods known by a skilled person in the art. By way of examples, density centrifugation (gradient) (Ficoll-Paque) can be used for isolating PBMCs. In other example, cell preparation tubes (CPTs) and SepMate tubes with freshly collected blood can be used for isolating PBMCs.

In some embodiments, the MNCs are PBMCs. By way of example, PBMC can comprise lymphocytes, monocytes, and dendritic cells. By way of example, lymphocytes can include T cells, B cells, and NK cells. In some embodiments, the MNCs as used herein are cultured or cryopreserved. In some embodiments, the process of culturing or cryopreserving the cells can include contacting the cells in culture with one or more antigens under suitable culture conditions to stimulate and expand antigen-specific T cells. In some embodiments, the one or more antigen can comprise one or more viral antigen. In some embodiments, the one or more antigen can comprise one or more tumor associated antigen. In other embodiments, the one or more antigen can comprise a combination of one or more viral antigen and one or more tumor associated antigen. For example, cultured or cryopreserved MNCs or PMBCs can be contacted with one adenovirus, a CTLA-4, and a gp100. In other embodiments, each antigen is a tumor associated antigen. In other embodiments, each antigen is a viral antigen. In other embodiments, at least one antigen is a viral antigen and at least one antigen is a tumor associated antigen.

In some embodiments, the process of culturing or cryopreserving the cells can include contacting the cells in culture with one or more epitope from one or more antigen under suitable culture conditions. In some embodiments, contacting the MNCs or PBMCs with one or more antigen, or one or more epitope from one or more antigen, stimulate and expand a polyclonal population of antigen-specific T cells from each of the respective donor's MNCs or PMBCs. In some embodiments, the antigen-specific T cell lines can be cryopreserved.

In some embodiments, the one or more antigen can be in the form of a whole protein. In some embodiments, the one or more antigen can be a pepmix comprising a series of overlapping peptides spanning part of or the entire sequence of each antigen. In some embodiments, the one or more antigen can be a combination of a whole protein and a pepmix comprising a series of overlapping peptides spanning part of or the entire sequence of each antigen.

In some embodiments, the culturing of the PBMCs or MNCs is in a vessel comprising a gas permeable culture surface. In one embodiment, the vessel is an infusion bag with a gas permeable portion or a rigid vessel. In one embodiment, the vessel is a GRex bioreactor. In one embodiment, the vessel can be any container, bioreactor, or the like, that are suitable for culturing the PBMCs or MNCs as described herein.

In some embodiments, the PBMCs or MNCs are cultured in the presence of one or more cytokine. In embodiments, the one or more cytokines cultured with the mononuclear cells and antigens is selected from the group consisting of IL-1, IL-2, IL-4, IL-6, IL-7, IL-12, IL-15, IL17, IL18 IL-21, and a combination thereof. In embodiments, the one or more cytokines cultured with the mononuclear cells and antigens is selected from the group consisting of IL-1, IL-4, IL-6, IL-7, IL-12, IL-15, IL17, IL18, IL-21, and a combination thereof. In embodiments, the one or more cytokines cultured with the mononuclear cells and antigens is selected from the group consisting of IL-1, IL-4, IL-6, IL-7, IL-12, IL-15, IL17, IL18, IL-21, and a combination thereof; and does not comprise IL-2. In some embodiments, the cytokine is IL-4. In some embodiments, the cytokine is IL-7. In some embodiments, the cytokine is IL-4 and IL-7. In some embodiments, the cytokine includes IL-4 and IL-7, but not IL-2. In some embodiments, the cytokine can be any combinations of cytokines that are suitable for culturing the PBMCs or MNCs as described herein.

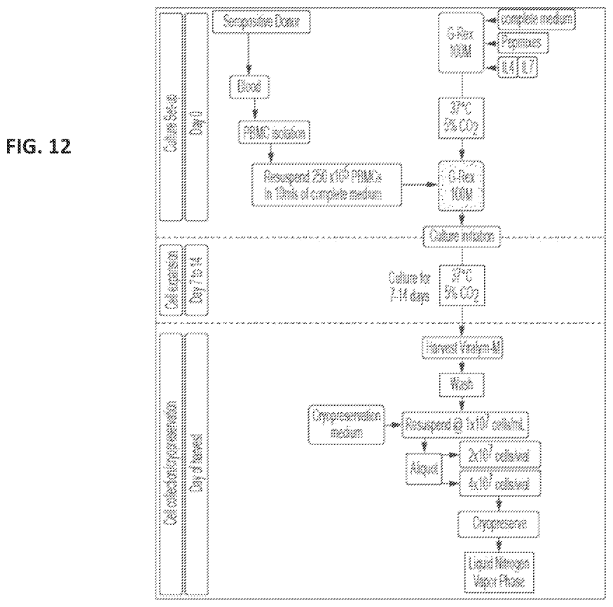

In some embodiments, culturing the MNCs or PBMCs can be in the presence of at least 2, 3, 4, 5, 6, 7, 8, 9, 10, 11, 12, 13, 14, 15, 16, 17, 18, 19, 20, or more different pepmixes. Pepmixes, a plurality of peptides, comprise a series of overlapping peptides spanning part of or the entire sequence of an antigen. In some embodiments, the MNCs or PBMCs can be cultured in the presence of a plurality of pepmixes. In this instance, each pepmix covers at least one antigen that is different than the antigen covered by each of the other pepmixes in the plurality of pepmixes. In some embodiments, at least 2, 3, 4, 5, 6, 7, 8, 9, 10, 11, 12, 13, 14, 15, 16, 17, 18, 19, 20 or more different antigens are covered by the plurality of pepmixes. In some embodiments, at least one antigen from at least 2 different viruses are covered by the plurality of pepmixes. FIG. 11 and FIG. 12 show an example of a general GMP manufacturing protocol of constructing the antigen-specific T cell lines. In some embodiments, a plurality of antigen specific T cell lines are individually prepared according to this method, where each respective line is prepared from donor material (e.g., MNCs or PBMCs) obtained from donors selected according to the present disclosure such that the selected donors have different HLA types from one another, and these lines are then pooled (optionally after being cryopreserved and subsequently thawed) to create a universal antigen specific T cell composition of the present invention. As is discussed further herein, in some embodiments, the plurality of antigen specific T cell lines are prepared from a sufficient number and diversity of donors to result in a universal antigen specific T cell composition (e.g., a UVST composition) containing at least one cell line matched on at least 2 HLA alleles with a large percentage of a given population. In some embodiments, the universal antigen specific T cell composition (e.g., a UVST composition) contains at least one cell line matched on at least 2 HLA alleles with >80%, >85%, >90%, or >95% of a given patient population (e.g., a patient population described herein).

In some embodiments, the pepmix comprises 15 mer peptides. In some embodiments, the pepmix comprises peptides that are suitable for the methods as described herein. In some embodiments, the peptides in the pepmix that span the antigen overlap in sequence by 8 amino acids, 9 amino acids, 10 amino acids, 11 amino acids, 12 amino acids, 13 amino acids, 14 amino acids, 15 amino acids. In some embodiments, the peptides in the pepmix that span the antigen overlap in sequence by 11 amino acids.

In some embodiments, the viral antigen in the one or more pepmixes is from a virus selected from EBV, CMV, Adenovirus, BK, JC virus, HHV6, RSV, Influenza, Parainfluenza, Bocavirus, Coronavirus, LCMV, Mumps, Measles, human Metapneumovirus, Parvovirus B, Rotavirus, merkel cell virus, herpes simplex virus, HPV, HIV, HTLV1, HHV8 and West Nile Virus, zika virus, ebola. In some embodiments, at least one pepmix covers an antigen from RSV, Influenza, Parainfluenza, Human meta-pneumovirus (HMPV). In some embodiments, the virus can be any suitable viruses.

In some embodiment, the influenza antigens can be influenza A antigen NP1. In some embodiment, the influenza antigens can be influenza A antigens MP1. In some embodiment, the influenza antigens can be a combination of NP1 and MP1. In some embodiments, the RSV antigens can be RSV N. In some embodiments, the RSV antigens can be RSV F. In some embodiments, the RSV antigens can be a combination of RSV N and F. In some embodiments, the hMPV antigens can be F. In some embodiments, the hMPV antigens can be N. In some embodiments, the hMPV antigens can be M2-1. In some embodiments, the hMPV antigens can be M. In some embodiments, the hMPV antigens can be a combination of F, N, M2-1, and M. In some embodiments, the PIV antigens can be M. In some embodiments, the PIV antigens can be HN. In some embodiments, the PIV antigens can be N. In some embodiments, the PIV antigens can be F. In some embodiments, the PIV antigens can be a combination of M, HN, N, and F.

In other embodiments, at least one pepmix covers an antigen from EBV, CMV, adenovirus, BK, and HHV6. In some embodiments, the EBV antigens are from LMP2, EBNA1, BZLF1, and a combination thereof. In some embodiments, the CMV antigens are from IE1, pp65, and a combination thereof. In some embodiments, the adenovirus antigens are from Hexon, Penton, and a combination thereof. In some embodiments, the BK virus antigens are from VP1, large T, and a combination thereof. In some embodiments, the HHV6 antigens are from U90, U 11, U14, and a combination thereof.

In some embodiments, the PBMCs or MNCs are cultured in the presence of pepmixes spanning influenza A antigen NP1 and Influenza A antigen MP1, RSV antigens N and F, hMPV antigens F, N, M2-1, and M, and PIV antigens M, HN, N, and F. In some embodiments, the PBMCs or MNCs are cultured in the presence of pepmixes spanning EBV antigens LMP2, EBNA1, and BZLF1, CMV antigens IE1 and pp65, adenovirus antigens Hexon and Penton, BK virus antigens VP1 and large T, and HHV6 antigens U90, U11, and U14. In some embodiments, the antigen specific T cells are tested for antigen-specific cytotoxicity.

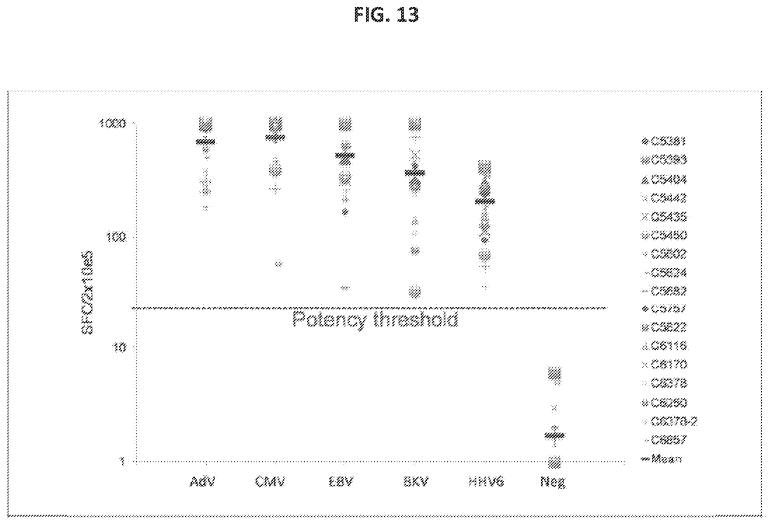

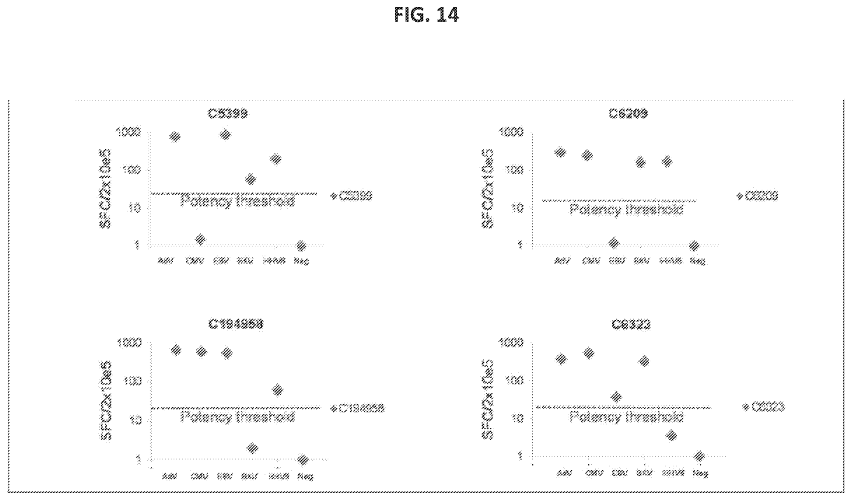

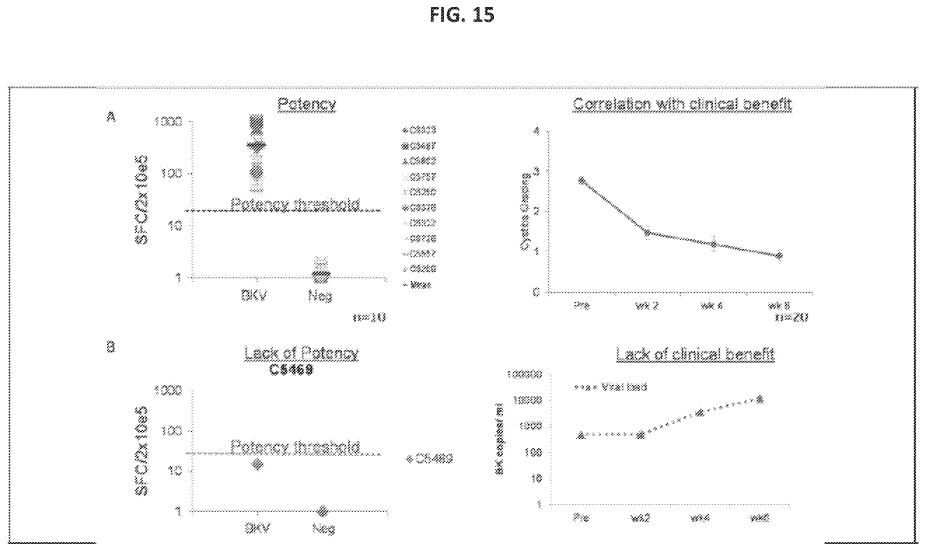

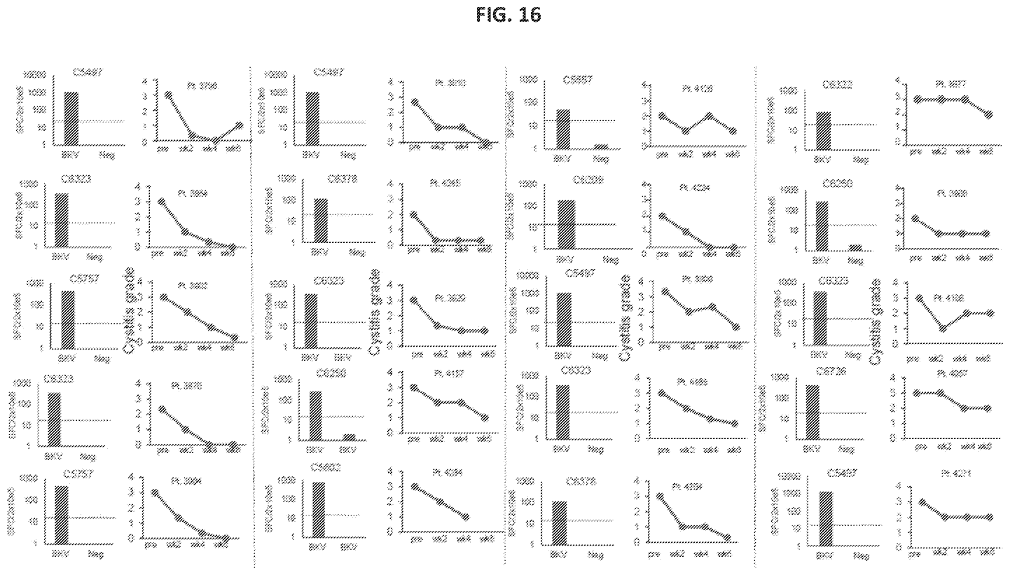

FIG. 13 shows the respective potency of the antigen-specific T cell lines against adenovirus, CMV, EBV, BKV, and HHV6 compared with the negative control, which is below the potency threshold. The T cells are specific for all five viruses as indicated by >30 SFC/2×10⁵ input VSTs, which is the threshold for discriminating between acceptance and rejection of a specific T cell line. The potency threshold of >30 SFC/2×10⁵ input VSTs was established based on experimental data using T cell lines generated from donors that were seronegative (based on serological screening) for one or more of the target viruses, which served as an internal negative control (FIG. 14).

The present disclosure provides methods of treating a disease or condition comprising administering to a patient one or more suitable antigen-specific T cell lines from the minibank as described herein. In embodiments, the disclosure includes methods for treating a disease or condition comprising administering to the patient a universal antigen-specific T cell product. In embodiments, the universal antigen-specific T cell product comprises cell lines from a minibank as described herein, wherein the cell lines from the minibank have been pooled together. In embodiments, the methods comprise administering to the patient a plurality of antigen-specific T cell lines from the minibank as described herein, wherein the cell lines form the minibank are administered to the patient in a single dosing session.

In some embodiments, the patient has received a haematopoietic stem cell transplant. In embodiments, the patient has received a haematopoietic stem cell transplant and the method comprises administering to the patient a universal antigen-specific T cell product comprising cell lines from the minibank as described herein, wherein the cell lines from the minibank have been pooled together. In some such embodiments, a portion of the cell lines from the minibank have been pooled together. In some such embodiments, all of the cell lines in the minibank have been pooled together. In embodiments, the patient has received a haematopoietic stem cell transplant and the method comprises administering to the patient a plurality of antigen-specific T cell lines from the minibank as described herein, wherein the cell lines form the minibank are administered to the patient in a single dosing session. Thus, in embodiments, the HLA type of the patient may or may not be determined prior to treatment.

In some embodiments, the disease treated is a viral infection. In some embodiments, the disease treated is cancer. In some embodiments, the condition treated is an immune deficiency. In some embodiments, the immune deficiency is primary immune deficiency. In embodiments, the patient has a viral infection, cancer, or an immune deficiency, and the method comprises administering to the patient a universal antigen-specific T cell product comprising cell lines from the minibank as described herein, wherein the cell lines from the minibank have been pooled together. In some such embodiments, a portion of the cell lines from the minibank have been pooled together. In some such embodiments, all of the cell lines in the minibank have been pooled together. In embodiments, the patient has a viral infection, cancer, or an immune deficiency, and the method comprises administering to the patient a plurality of antigen-specific T cell lines from the minibank as described herein, wherein the cell lines form the minibank are administered to the patient in a single dosing session. Thus, in embodiments, the HLA type of the patient may or may not be determined prior to treatment.

In some embodiments, the patient is immunocompromised. As used herein, immunocompromised means having a weakened immune system. For example, patients who are immunocompromised have a reduced ability to fight infections and other diseases. In some embodiments, the patient is immunocompromised due to a treatment the patient received to treat the disease or condition or another disease or condition. In some embodiments, the cause of immunocompromised is due to age. In one embodiment, the cause of immunocompromised is due to young age. In one embodiment, the cause of immunocompromised is due to old age. In some embodiments, the patient is in need of a transplant therapy.

The present disclosure provides methods of selecting a first antigen-specific T cell line from the minibank or from a minibank comprised in the donor bank, for administration in an allogeneic T cell therapy to a patient who has received transplanted material from a transplant donor in a transplant procedure. In one embodiment, the administration is for treatment of a viral infection. In one embodiment, the administration is for treatment a tumor. In one embodiment, the administration is for treatment of a viral infection and tumor. In one embodiment, the administration is for primary immune deficiency prior to transplant. In some embodiments, the transplanted material comprises stem cells. In some embodiments, the transplanted material comprises a solid organ. In some embodiments, the transplanted material comprises bone marrow. In some embodiments, the transplanted material comprises stem cells, a solid organ, and bone marrow.

The present disclosure provides methods of constructing a donor bank made up of a plurality of minibanks of antigen specific T cell lines. As used herein, “a plurality of minibanks” means more than one minibank of antigen specific T cell lines. For example, the donor bank can comprise two, three, four, five, or six minibanks. In some embodiments, constructing a donor bank comprises the steps and procedures for constructing a first donor minibank as described herein. The steps and procedures includes conducting one or more second rounds to construct one or more second minibanks. In some embodiments, prior to starting each second round of the method, a new donor pool can be generated. In some embodiments, the new donor pool comprises the first donor pool less any greatest matched donors removed from the first and any prior second rounds of the method as disclosed herein. In some embodiments, the new donor pool comprises an entirely new population of potential donors not included in the first donor pool. In some embodiments, the new donor pool comprises the first donor pool less any greatest matched donors removed from the first and any prior second rounds of the method as disclosed herein as well as an entirely new population of potential donors not included in the first donor pool. In some embodiments, generating a new donor pool comprises reconstituting the first plurality of prospective patients from the first prospective patient population by returning all prospective patients that had been previously removed from the first and any prior second rounds of the method as described herein. In some embodiments, after each round of selecting and removing, MNCs are isolated from the blood obtained from each respective donor included in the donor minibank. In some embodiments, the MNCs are cultured and contacted with one or more antigen, or one or more epitope from one or more antigen, under suitable culture condition. In some embodiments, the MNCs are stimulated and expand a polyclonal population of antigen-specific T cells. In some embodiments, a plurality of T cell lines are produced. The methods of culturing, contacting of antigens, and preparing pepmixes are the same as the processes for constructing the first donor minibank as described herein.

The present disclosure provides methods of treating a disease or condition comprising administering to a patient two or more suitable antigen-specific T cell lines from the donor bank comprising a plurality of minibanks of antigen-specific T cell lines as described herein. In various embodiments, the two or more suitable antigen-specific T cell lines from the donor bank are administered to the patient in a single dosing session. In some embodiments, all of the antigen-specific T cell lines from the donor bank are administered to the patient, optionally in the single dosing session.

Inflammatory response can be detected by observing one or more symptom or sign of (i) constitutional symptoms selected from fever, rigors, headache, malaise, fatigue, nausea, vomiting, arthralgia; (ii) vascular symptoms including hypotension; (iii) cardiac symptoms including arrhythmia; (iv) respiratory compromise; (v) renal symptoms including kidney failure and uremia; and (vi) laboratory symptoms including coagulopathy and a hemophagocytic lymphohistiocytosis-like syndrome. In some embodiments, inflammatory response can be detected by observing any signs that are known or common.

In some embodiments, the treatment efficacy is measured post-administration of the plurality of antigen specific T cell lines and/or universal antigen-specific T cell product. In other embodiments, the treatment efficacy is measured based on viremic resolution of infection. In other embodiments, the treatment efficacy is measured based on viruric resolution of infection. In other embodiments, the treatment efficacy is measured based on resolution of viral load in a sample from the patient. In other embodiments, the treatment efficacy is measured based on viremic resolution of infection, viruric resolution of infection, and resolution of viral load in a sample from the patient. In some embodiments, the treatment efficacy is measured by monitoring viral load detectable in the peripheral blood of the patient. In some embodiments, the treatment efficacy comprises resolution of macroscopic hematuria. In some embodiments, the treatment efficacy comprises reduction of hemorrhagic cystitis symptoms as measured by the CTCAE-PRO or similar assessment tool that examines patient and/or clinician-reported outcomes. In some embodiments, the treatment efficacy is measured based on tumor size reduction post-administration of the plurality of antigen specific T cell lines and/or universal antigen-specific T cell product when the treatment is against a cancer. In some embodiments, the treatment efficacy is measured by monitoring markers of disease burden detectable in the peripheral blood/serum of the patient. In some embodiments, the treatment efficacy is measured by monitoring markers of tumor lysis detectable in the peripheral blood/serum of the patient. In some embodiments, the treatment efficacy is measured by monitoring tumor status via imaging studies.

In embodiments, the sample is selected from a tissue sample from the patient. In embodiments, the sample is selected from a fluid sample from the patient. In embodiments, the sample is selected from cerebral spinal fluid (CSF) from the patient. In embodiments, the sample is selected from BAL from the patient. In embodiments, the sample is selected from stool from the patient.

The present disclosure provides methods of identifying suitable donors for use in constructing a first donor minibank of antigen-specific T cells. In some embodiments, the methods comprise determining or having determined the HLA type of each of a first plurality of potential donors from a first donor pool. In some embodiments, the methods comprise determining or having determined the HLA type of each of a first plurality of prospective patients from a first prospective patient population. In some embodiments, the methods comprise comparing the HLA type of each of a first plurality of potential donors from a first donor pool with each of a first plurality of prospective patients from a first prospective patient population. In some embodiments, the methods comprise determining a first greatest matched donor, defined as the donor from the first donor pool that has 2 or more allele matches with the greatest number of patients in the first plurality of prospective patients. In some embodiments, the methods comprise selecting the first greatest matched donor for inclusion in a first donor minibank.

In some embodiments, the methods comprise removing from the first donor pool the first greatest matched donor thereby generating a second donor pool consisting of each of the first plurality of potential donors from the first donor pool except for the first greatest matched donor. In some embodiments, the methods comprise removing from the first plurality of prospective patients each prospective patient that has 2 or more allele matches with the first greatest matched donor. A second plurality of prospective patients consisting of each of the first plurality of prospective patients except for each prospective patient that has 2 or more allele matches with the first greatest matched donor are then generated. In some embodiments, the methods comprise repeating the steps and processes as described herein (e.g. comparing the HLA type of each of a first plurality of potential donors from a first donor pool with each of a first plurality of prospective patients from a first prospective patient population, determining and selecting the greatest match for the donor minibank, removing from the first donor pool the first greatest matched donor and the prospective patients) for all donors and prospective patients that have not already been removed.

Each repeating process allows the selection of an additional greatest matched donor and the removal of each prospective patient that has 2 or more allele matches with that subsequent greatest matched donor. The processes as described herein sequentially increases the number of selected greatest matched donors in the first donor minibank by 1 following each cycle of the method. The processes then depletes the number of the plurality of prospective patients in the patient population following each cycle of the method in accordance with their HLA matching to the selected greatest matched donors. The processes are repeated until a desired percentage (e.g. less than 5%) of the first prospective patient population remains in the plurality of prospective patients or until no donors remain in the donor pool. In some embodiments, the processes are repeated until more than 95% of the prospective patients are matched and covered.

In particular embodiments, such methods of identifying suitable donors for use in constructing a first donor minibank of antigen-specific T cells are similarly utilized in identifying suitable donors for use in constructing a universal antigen-specific T cell composition. For example, in some embodiments, the methods comprise determining or having determined the HLA type of each of a first plurality of potential donors from a first donor pool. In some embodiments, the methods comprise determining or having determined the HLA type of each of a first plurality of prospective patients from a first prospective patient population. In some embodiments, the methods comprise comparing the HLA type of each of a first plurality of potential donors from a first donor pool with each of a first plurality of prospective patients from a first prospective patient population. In some embodiments, the methods comprise determining a first greatest matched donor, defined as the donor from the first donor pool that has 2 or more allele matches with the greatest number of patients in the first plurality of prospective patients. In some embodiments, the methods comprise selecting the first greatest matched donor for inclusion in the universal antigen-specific T cell composition.

In some embodiments, the methods comprise removing from the first donor pool the first greatest matched donor thereby generating a second donor pool consisting of each of the first plurality of potential donors from the first donor pool except for the first greatest matched donor. In some embodiments, the methods comprise removing from the first plurality of prospective patients each prospective patient that has 2 or more allele matches with the first greatest matched donor. A second plurality of prospective patients consisting of each of the first plurality of prospective patients except for each prospective patient that has 2 or more allele matches with the first greatest matched donor are then generated. In some embodiments, the methods comprise repeating the steps and processes as described herein (e.g. comparing the HLA type of each of a first plurality of potential donors from a first donor pool with each of a first plurality of prospective patients from a first prospective patient population, determining and selecting the greatest match for the universal antigen-specific T cell composition, removing from the first donor pool the first greatest matched donor and the prospective patients) for all donors and prospective patients that have not already been removed.

Each repeating process allows the selection of an additional greatest matched donor and the removal of each prospective patient that has 2 or more allele matches with that subsequent greatest matched donor. The processes as described herein sequentially increases the number of selected greatest matched donors for use in the universal antigen-specific T cell composition by 1 following each cycle of the method. The processes then depletes the number of the plurality of prospective patients in the patient population following each cycle of the method in accordance with their HLA matching to the selected greatest matched donors. The processes are repeated until a desired percentage (e.g. less than 5%) of the first prospective patient population remains in the plurality of prospective patients or until no donors remain in the donor pool. In some embodiments, the processes are repeated until more than 95% of the prospective patients are matched and covered. The donor cells may then be processed to produce antigen specific T cell lines, e.g., using a method known in the art or disclosed herein. In some embodiments, such antigen specific T cell lines may then be pooled together to produce a universal antigen-specific T cell composition described herein.

Viral infections are a serious cause of morbidity and mortality after allogenic hematopoietic stem cell transplantation (allo-HSCT). Viral reactivation is likely to occur during the relative or absolute immunodeficiency of aplasia and during immunosuppressive therapy after allo-HSCT. Infections associated with viral pathogens including cytomegalovirus (CMV), BK virus (BKV), and adenovirus (AdV), have become increasingly problematic following allo-HSCT and are associated with significant morbidity and mortality.

Among the common infections, CMV remains the most clinically significant infection after allogeneic hematopoietic stem cell transplant (HSCT). Center for International Blood and Marrow Transplant Research (CIBMTR) data show that early post-transplant CMV reactivation occurs in over 30% of CMV seropositive HSCT recipients and can result in colitis, retinitis, pneumonitis, and death. Although antiviral agents including ganciclovir, valganciclovir, letermovir, foscarnet and cidofovir have been used both prophylactically and therapeutically, they are not always effective and are associated with significant toxicities including bone marrow suppression, renal toxicity, and ultimately, non-relapse mortality. Since immune reconstitution remains paramount to infection control, the adoptive transfer of ex vivo expanded/isolated CMV-specific T cells (CMVSTs) has emerged as an effective means of providing antiviral benefit.

Early immunotherapies targeting CMV focused on stem cell donor-derived T cell products, which proved both safe and effective in a series of academic Phase 1/II studies spanning more than 20 years. However, the personalized nature of the therapy as well the requirement for virus-immune donors (an important issue given the benefits of using younger donors that are more likely virus-naive) have emerged as barriers that preclude broad implementation. Thus, more recently, partially HLA-matched third party-derived virus-specific T cells (VSTs), which can be prepared prospectively and banked in advance of clinical need, have been investigated as a therapeutic modality. These VSTs have proved safe and effective against a spectrum of viruses including Epstein-Barr virus, CMV, adenovirus, HHV6 and BK virus in >150 HSCT or solid organ transplant (SOT) recipients with drug-refractory infections/disease. These studies prompted interest in advancing “off the shelf” virus-specific T cells towards pivotal studies and subsequent commercialization, with the remaining questions relating to (i) the number of cell lines required to accommodate the diverse transplant population, and (ii) establishing criteria for line selection to assure clinical benefit.

In addition, the emergence of infections caused by reactivation of latent BKV, a member of the Polyomavirus family, causes severe clinical disease in HSCT patients. The primary clinical manifestation of BKV infection following allogeneic HSCT is hemorrhagic cystitis (BK-HC). This occurs in up to 25% of allogeneic HSCT recipients and manifests as gross hematuria with severe, often debilitating, abdominal pain requiring continuous narcotic infusions. In healthy individuals, T cell immunity defends against viruses. In allo-HSCT recipients the use of potent immunosuppressive regimens (and subsequent associated immune compromise) leaves patients susceptible to severe viral infections.

Respiratory viral infections due to community-acquired respiratory viruses (CARVs) including respiratory syncytial virus (RSV), influenza, parainfluenza virus (PIV) and human metapneumovirus (hMPV) are detected in up to 40% of allogeneic hematopoietic stem cell transplant (allo-HSCT) recipients, in whom they may cause severe disease such as bronchiolitis and pneumonia that can be fatal. RSV induced bronchiolitis is the most common reason for hospital admission in children less than 1 year, while the Center for Disease Control (CDC) estimates that, annually, Influenza accounts for up to 35.6 million illnesses worldwide, between 140,000 and 710,000 hospitalizations, annual costs of approximately $87.1 billion in disease management in the US alone and between 12,000 and 56,000 deaths.

The present disclosure provides restoration of T cell immunity by the administration of ex vivo expanded, non-genetically modified, virus-specific T cells (VSTs) to control viral infections and eliminate symptoms for the period until the transplant patient's own immune system is restored. Without wishing to be bound by any theories, VSTs recognize and kill virus-infected cells via their native T cell receptor (TCR), which binds to major histocompatibility complex (MHC) molecules expressed on target cells that present virus-derived peptides. In particular embodiments, the present disclosure provides restoration of T cell immunity by the administration of UVSTs described herein. Any of the VST compositions described herein may also be formulated into a UVST composition simply by pooling together two or more VST compositions that have sufficient HLA diversity (e.g., wherein said VST lines are derived from donor material originating from at least two separate donors, and wherein the HLA type of each donor differs from at least one of the other donors on at least one HLA allele).

In some embodiments, VSTs from peripheral blood mononuclear cells (PBMCs) procured from healthy, pre-screened, seropositive donors, which are available as a partially HLA-matched “off-the-shelf” product. In some embodiments, UVSTs from peripheral blood mononuclear cells (PBMCs) procured from healthy, pre-screened, seropositive donors, which are available as a partially HLA-matched “off-the-shelf” product. In some embodiments, the UVSTs as described herein respond to at least EBV, CMV, Adenovirus, BK virus, HHV6, RSV, Influenza, Parainfluenza, Bocavirus, Coronavirus, LCMV, Mumps, Measles, Metapneumovirus, Parvovirus B, Rotavirus, West Nile Virus, Spanish influenza, or a combination thereof. In some embodiments, the VSTs as described herein respond to at least EBV, CMV, AdV, BKV, and HHV6. In some embodiments, the UVSTs as described herein respond to at least RSV, Influenza, Parainfluenza, and Metapneumovirus. In some embodiments, the UVSTs as described herein respond to at least a Coronavirus (e.g., SARS-CoV2). In some embodiments, the UVSTs as described herein respond to at least RSV, Influenza, Parainfluenza, Metapneumovirus and a Coronavirus (e.g., SARS-CoV2). In some embodiments, the UVSTs as described herein respond to at least Hepatitis B (HBV). In some embodiments, the UVSTs as described herein respond to at least Human Herpesvirus-8.

In some embodiments, the VSTs are designed to circulate only until the patient regain immunocompetence following HSCT engraftment and immune system repopulation. Without wishing to be bound by theories, the VSTs and methods as described herein are “immunologic bridge therapy” that provides an immunocompromised patient with T cell immunity until the patient engrafts and can mount an endogenous immune response.

In embodiments, the method provided herein comprise prophylactically administering to a patient a universal antigen-specific T cell product and/or two or more antigen-specific T cell lines generated from donors of diverse HLA types (e.g., two or more antigen-specific T cell lines from a donor minibank or donor bank provided herein). In embodiments, the administering prophylactically of the two or more antigen-specific T cell lines is performed in a single dosing session. In embodiments, methods comprise prophylactically administering UVST. The prophylactic administration is such that the patient does not show evidence of an active viral infection or of reactivation of the latent virus when the T cell product is administered. For example, in embodiments, the patient is administered a UVST product specific for one or more viruses, wherein the patient does not have an active infection with respect to the one or more viruses, or wherein the patient does not have any active viral infection. In embodiments, the patient has no detectable viremia or viruria when the T cell line is administered.

In embodiments, the universal antigen-specific T cell product and/or two or more antigen-specific T cell lines generated from donors of diverse HLA types (e.g., two or more antigen-specific T cell lines from a donor minibank or donor bank provided herein) are capable of persisting, and retaining the ability to expand, in the recipient patient in the absence of an active viral infection for which the T cells have specificity. Further, in embodiments, the T cell lines are capable of persisting for several weeks after administration to the patient and then expanding immediately upon infection with or reactivation of one or more virus for which they are specific. In some embodiments, the persistence of the universal antigen-specific T cell product and/or two or more antigen-specific T cell lines generated from donors of diverse HLA types (e.g., two or more antigen-specific T cell lines from a donor minibank or donor bank provided herein) in a patient is increased by administering to the patient a booster vaccine containing one or more of the antigens utilized in making the universal antigen-specific T cell product and/or the two or more antigen-specific T cell lines. Thus, the present disclosure provides a highly efficient method for preventing or controlling a viral infection or the reactivation of a latent virus in an allogeneic setting, using a universal antigen-specific T cell therapy product and/or T cell lines from a donor minibank or donor bank as provided herein. In particular, the methods and compositions provided herein provide an immediately available, safe, and effective protection against dangerous viral infections in patients at high risk, such as patients who are recipients of allogeneic-HSCT.

### Viral Antigens

In some embodiments of the disclosure, the generated antigen specific T cells are provided to an individual that has or is at risk of having a pathogenic infection, including a viral, bacterial, or fungal infection. In some embodiments of the disclosure, the generated universal antigen specific T cell compositions are provided to an individual that has or is at risk of having a pathogenic infection, including a viral, bacterial, or fungal infection. The individual may or may not have a deficient immune system. In some cases, the individual has a viral, bacterial, or fungal infection following organ or stem cell transplant (including hematopoietic stem cell transplantation), or has cancer or has been subjected to cancer treatment, for example. In some cases the individual has infection following an acquired immune system deficiency.

The infection in the individual may be of any kind, but in specific embodiments the infection is the result of one or more viruses. The pathogenic virus may be of any kind, but in specific embodiments it is from one of the following families: Adenoviridae, Picornaviridae, Herpesviridae, Hepadnaviridae, Flaviviridae, Retroviridae, Orthomyxoviridae, Paramyxoviridae, Papovaviridae, Polyomavirus, Rhabdoviridae, or Togaviridae. In some embodiments, the virus produces antigens that are immunodominant or subdominant or produces both kinds. In specific cases, the virus is selected from the group consisting of EBV, CMV, Adenovirus, BK virus, HHV6, RSV, Influenza, Parainfluenza, Bocavirus, Coronavirus, LCMV, Mumps, Measles, Metapneumovirus, Parvovirus B, Rotavirus, West Nile Virus, Spanish influenza, and a combination thereof.

In some aspects the infection is the result of a pathogenic bacteria, and the present invention is applicable to any type of pathogenic bacteria. Exemplary pathogenic bacteria include at least Mycobacterium tuberculosis, Mycobacterium leprae, Clostridium botulinum, Bacillus anthracis, Yersinia pestis, Rickettsia prowazekii, Streptococcus, Pseudomonas, Shigella, Campylobacter, and Salmonella.

In some aspects the infection is the result of a pathogenic fungus, and the present invention is applicable to any type of pathogenic fungus. Exemplary pathogenic fungi include at least Candida, Aspergillus, Cryptococcus, Histoplasma, Pneumocystis, or Stachybotrys. In some embodiments, viral antigens can be any antigens that are suitable for the use as described in the present disclosure.

### Tumor Antigens

In embodiments wherein TAA-specific or multiTAA-specific antigen specific T cells are employed for the treatment and/or prevention of cancer, a variety of TAA may be targeted. In embodiments wherein TAA-specific or multiTAA-specific universal antigen specific T cell compositions are employed for the treatment and/or prevention of cancer, a variety of TAA may be targeted. Tumor antigens are substances produced in tumor cells that trigger an immune response in a host. As used herein, the terms “tumor antigen,” “tumor associated antigen,” and “TAA” are used interchangeably. Thus, these terms encompasses both tumor specific antigens (which are antigens that are expressed only on tumor cells only, but not on healthy cells) and tumor associated antigens, which are upregulated/overexpressed on tumor cells, but are not specific to tumor cells.

Exemplary tumor antigens include at least the following: carcinoembryonic antigen (CEA) for bowel cancers; CA-125 for ovarian cancer; MUC-1 or epithelial tumor antigen (ETA) or CA15-3 for breast cancer; tyrosinase or melanoma-associated antigen (MAGE) for malignant melanoma; and abnormal products of ras, p53 for a variety of types of tumors; alphafetoprotein for hepatoma, ovarian, or testicular cancer; beta subunit of hCG for men with testicular cancer; prostate specific antigen for prostate cancer; beta 2 microglobulin for multiple myelom and in some lymphomas; CA19-9 for colorectal, bile duct, and pancreatic cancer; chromogranin A for lung and prostate cancer; TA90 for melanoma, soft tissue sarcomas, and breast, colon, and lung cancer. Examples of tumor antigens are known in the art, for example in Cheever et al., 2009, which is incorporated by reference herein in its entirety.

Specific examples of tumor antigens include at least CEA, MHC, CTLA-4, gp100, mesothelin, PD-L1, TRP1, CD40, EGFP, Her2, TCR alpha, trp2, TCR, MUC1, cdr2, ras, 4-1BB, CT26, GITR, OX40, TGF-α. WT1, MUC1, LMP2, HPV E6 E7, EGFRvIII, HER-2/neu, MAGE A3, p53 nonmutant, NY-ESO-1, PSMA, GD2, Melan A/MART1, Ras mutant, gp 100, p53 mutant, Proteinase3 (PR1), bcr-abl, Tyrosinase, Survivin, PSA, hTERT, EphA2, PAP, ML-IAP, AFP, EpCAM, ERG (TMPRSS2 ETS fusion gene), NA17, PAX3, ALK, Androgen receptor, Cyclin B1, Polysialic acid, MYCN, RhoC, TRP-2, GD3, Fucosyl GM1, Mesothelin, PSCA, MAGE A1, sLe(a), CYP1B1, PLAC1, GM3, BORIS, Tn, GloboH, ETV6-AML, NY-BR-1, RGS5, SART3, STn, Carbonic anhydrase IX, PAX5, OY-TES1, Sperm protein 17, LCK, HMWMAA, AKAP-4, SSX2, XAGE 1, B7H3, Legumain, Tie 2, Page4, VEGFR2, MAD-CT-1, FAP, PDGFR-β, MAD-CT-2, and Fos-related antigen 1, for example. In some embodiments, tumor antigens can be any antigens that are suitable for the use as described in the present disclosure.

Exemplary cancers include cancers that are solid tumors or hematological malignancies. In embodiments, the cancer is selected from the group consisting of lung cancer (e.g., non-small cell lung cancer, small cell lung cancer, squamous cell lung cancer, or large cell lung cancer), head and neck cancer, mesothelioma (e.g., malignant pleural mesothelioma), pancreatic cancer (e.g., pancreatic ductal adenocarcinoma, or metastatic pancreatic ductal adenocarcinoma (PDA)), esophageal cancer, ovarian cancer, cervical cancer, fallopian tube cancer, breast cancer, gastric cancer, colorectal cancer, or bladder cancer, melanoma, or any combination thereof. In embodiments, the cancer is a leukemia or lymphoma. In embodiments, the cancer is selected from the group consisting of chronic lymphocytic leukemia (CLL), mantle cell lymphoma (MCL), multiple myeloma, acute lymphoid leukemia (ALL), Hodgkin lymphoma, B-cell acute lymphoid leukemia (BALL), T-cell acute lymphoid leukemia (TALL), small lymphocytic leukemia (SLL), B cell prolymphocytic leukemia, blastic plasmacytoid dendritic cell neoplasm, Burkitt's lymphoma, diffuse large B cell lymphoma (DLBCL), DLBCL associated with chronic inflammation, chronic myeloid leukemia, myeloproliferative neoplasms, follicular lymphoma, pediatric follicular lymphoma, hairy cell leukemia, small cell- or a large cell-follicular lymphoma, malignant lymphoproliferative conditions, MALT lymphoma (extranodal marginal zone lymphoma of mucosa-associated lymphoid tissue), Marginal zone lymphoma, myelodysplasia, myelodysplastic syndrome, non-Hodgkin lymphoma, plasmablastic lymphoma, plasmacytoid dendritic cell neoplasm, Waldenstrom macroglobulinemia, splenic marginal zone lymphoma, splenic lymphoma/leukemia, splenic diffuse red pulp small B-cell lymphoma, hairy cell leukemia-variant, lymphoplasmacytic lymphoma, a heavy chain disease, plasma cell myeloma, solitary plasmocytoma of bone, extraosseous plasmocytoma, nodal marginal zone lymphoma, pediatric nodal marginal zone lymphoma, primary cutaneous follicle center lymphoma, lymphomatoid granulomatosis, primary mediastinal (thymic) large B-cell lymphoma, intravascular large B-cell lymphoma, ALK+ large B-cell lymphoma, large B-cell lymphoma arising in HHV8-associated multicentric Castleman disease, primary effusion lymphoma, B-cell lymphoma, acute myeloid leukemia (AML), and unclassifiable lymphoma.

### Generation of Pepmix Libraries

In some embodiments of the invention, a library of peptides is provided to PBMCs ultimately to generate antigen specific T cells. The library in particular cases comprises a mixture of peptides (“pepmixes”) that span part or all of the same antigen. Pepmixes utilized in the invention may be from commercially available peptide libraries made up of peptides that are 15 amino acids long and overlapping one another by 11 amino acids, in certain aspects. In some cases, they may be generated synthetically. Examples include those from JPT Technologies (Springfield, VA) or Miltenyi Biotec (Auburn, CA). In particular embodiments, the peptides are at least 7, 8, 9, 10, 11, 12, 13, 14, 15, 16, 17, 18, 19, 20, 21, 22, 23, 24, 25, 26, 27, 28, 29, 30, 31, 32, 33, 34, or 35 or more amino acids in length, for example, and in specific embodiments there is overlap of at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, or 34 amino acids in length, for example.

In some embodiments, the amino acids as used in the pepmixes have at least 85%, at least 86%, at least 87%, at least 88%, at least 89%, at least 90%, at least 91%, at least 92%, at least 93%, at least 94%, at least 95%, at least 96%, at least 97%, at least 98%, at least 99, at least 99.9% purity, inclusive of all ranges and subranges therebetween. In some embodiments, the amino acids as used here in the pepmixes have at least 90% purity.

The mixture of different peptides may include any ratio of the different peptides, although in some embodiments each particular peptide is present at substantially the same numbers in the mixture as another particular peptide. The methods of preparing and producing pepmixes for multiviral antigen-specific T cells with broad specificity is described in US2018/0187152, which is incorporated by reference in its entirety.

### Universal Virus-Specific T Cell Compositions

The present disclosure includes polyclonal virus-specific T cell compositions, generated from seropositive donors (e.g., selected via the donor selection methods disclosed herein), with specificity against clinically significant viruses. The present disclosure also includes universal antigen specific T cell compositions, as disclosed herein, comprising a plurality of such polyclonal virus-specific T cell compositions, generated from a plurality of such donors. In some embodiments, the clinically significant viruses can include but are not limited to EBV, CMV, AdV, BKV and HHV6. In some embodiments, the viral antigens span immunogenic antigens from BK virus (VP1 and large T), AdV (Hexon and Penton), CMV (IE1 and pp65), EBV (LMP2, EBNA1, BZLF1) and HHV6 (U90, U11 and U14).

The present disclosure provides a composition comprising a polyclonal population of antigen specific T cells. In some embodiments, the polyclonal population of antigen specific T cells can recognize a plurality of viral antigens. In some embodiments, the plurality of viral antigens can comprise at least one first antigen from parainfluenza virus type 3 (PIV). In some embodiments, the plurality of viral antigens can comprise at least one second antigen from one or more second virus. In some embodiments, the present disclosure also includes universal antigen specific T cell compositions, as disclosed herein, comprising a plurality of such antigen specific T cells, generated from a plurality of such donors.

In some embodiments, polyclonal virus-specific T cell compositions have specificity against any clinically significant or relevant viruses. For example, polyclonal virus-specific T cell compositions can comprise VSTs with specificity for viral antigens including CMV, BKV, PIV, and RSV. In some embodiments, UVST compositions have specificity against any clinically significant or relevant viruses. For example, UVST compositions can comprise VSTs with specificity for viral antigens including CMV, BKV, PIV, and RSV.

In some embodiments, the first antigen can be PIV antigen M. In some embodiments, the first antigen can be PIV antigen HN. In some embodiments, the first antigen can be PIV antigen N. In some embodiments, the first antigen can be PIV antigen F. In some embodiments, the first antigen can be any combinations of PIV antigen M, PIV antigen HN, PIV antigen N, and PIV antigen F. In some embodiments, the composition can comprise VSTs with specificity for 1 first antigen. In some embodiments, the composition can comprise VSTs with specificity for 2 first antigens. In some embodiments, the composition can comprise VSTs with specificity for 3 first antigens. In some embodiments, the composition can comprise VSTs with specificity for 4 first antigens. In some embodiments, the 4 first antigens can comprise PIV antigen M, PIV antigen HN, PIV antigen N, and PIV antigen F.

In some embodiments, the one or more second virus can be respiratory syncytial virus (RSV). In some embodiments, the one or more second virus can be Influenza. In some embodiments, the one or more second virus can be human metapneumovirus (hMPV). In some embodiments, the one or more second virus can comprises respiratory syncytial virus (RSV), Influenza, and human metapneumovirus. In some embodiments, the one or more second virus can consist of respiratory syncytial virus (RSV), Influenza, and human metapneumovirus. In some embodiments, the one or more second virus can be selected from any suitable viruses as described herein. In some embodiments, UVST compositions have specificity against one or more of RSV, Influenza, or hMPV.

In some embodiments, the composition can comprise VSTs with specificity for two or three second viruses. In some embodiments, the composition can comprise VSTs with specificity for three second viruses. In some embodiments, the three second viruses can comprise influenza, RSV, and hMPV. In some embodiments, the composition can comprise VSTs with specificity for at least two second antigens per each second virus. In some embodiments, the composition comprises VSTs with specificity for 1 second antigen. In some embodiments, the composition comprises VSTs with specificity for 2 second antigens. In some embodiments, the composition comprises VSTs with specificity for 3 second antigens. In some embodiments, the composition comprises VSTs with specificity for 4 second antigens. In some embodiments, the composition comprises VSTs with specificity for 5 second antigens. In some embodiments, the composition comprises 6 second antigens. In some embodiments, the composition comprises VSTs with specificity for 7 second antigens. In some embodiments, the composition comprises VSTs with specificity for 8 second antigens. In some embodiments, the composition comprises VSTs with specificity for 9 second antigens. In some embodiments, the composition comprises VSTs with specificity for 10 second antigens. In some embodiments, the composition comprises VSTs with specificity for 11 second antigens. In some embodiments, the composition comprises VSTs with specificity for 12 second antigens. In some embodiments, the composition comprises VSTs with specificity for any numbers of second antigens that would be suitable for the compositions as described herein.

In some embodiments, the second antigen can be influenza antigen NP1. In some embodiments, the second antigen can be influenza antigen MP1. In some embodiments, the second antigen can be RSV antigen N. In some embodiments, the second antigen can be RSV antigen F. In some embodiments, the second antigen can be hMPV antigen M. In some embodiments, the second antigen can be hMPV antigen M2-1. In some embodiments, the second antigen can be hMPV antigen F. In some embodiments, the second antigen can be hMPV antigen N. In some embodiments, the second antigen can be any combinations of influenza antigen NP1, influenza antigen MP1, RSV antigen N, RSV antigen F, hMPV antigen M, hMPV antigen M2-1, hMPV antigen F, and hMPV antigen N.

In some embodiments, the second antigen comprises influenza antigen NP1. In some embodiments, the second antigen comprises influenza antigen MP1. In some embodiments, In some embodiments, the second antigen comprises both influenza antigen NP1 and influenza antigen MP1. In some embodiments, the second antigen comprises RSV antigen N. In some embodiments, the second antigen comprises RSV antigen F. In some embodiments, the second antigen comprises both RSV antigen N RSV antigen F.

In some embodiments, the second antigen comprises hMPV antigen M. In some embodiments, the second antigen comprises hMPV antigen M2-1. In some embodiments, the second antigen comprises hMPV antigen F. In some embodiments, the second antigen comprises hMPV antigen N. In some embodiments, the second antigen comprises combinations of hMPV antigen M, hMPV antigen M2-1, hMPV antigen F, and hMPV antigen N.

In some embodiments, the second antigen comprises each of influenza antigen NP1, influenza antigen MP1, RSV antigen N, RSV antigen F, hMPV antigen M, hMPV antigen M2-1, hMPV antigen F, hMPV antigen N. In some embodiments, the plurality of antigens comprise PIV antigen M, PIV antigen HN, PIV antigen N, PIV antigen F, influenza antigen NP1, influenza antigen MP1, RSV antigen N, RSV antigen F, hMPV antigen M, hMPV antigen M2-1, hMPV antigen F, and hMPV antigen N. In some embodiments, the plurality of antigens consist of PIV antigen M, PIV antigen HN, PIV antigen N, PIV antigen F, influenza antigen NP1, influenza antigen MP1, RSV antigen N, RSV antigen F, hMPV antigen M, hMPV antigen M2-1, hMPV antigen F, and hMPV antigen N. In some embodiments, the plurality of antigens consist essentially of PIV antigen M, PIV antigen HN, PIV antigen N, PIV antigen F, influenza antigen NP1, influenza antigen MP1, RSV antigen N, RSV antigen F, hMPV antigen M, hMPV antigen M2-1, hMPV antigen F, and hMPV antigen N. In some embodiments, the second antigen can comprise any suitable antigens for the compositions as described herein.

In some embodiments, the clinically significant viruses can include but are not limited to HHV8. In some embodiments, the viral antigens span immunogenic antigens from HHV8. In some embodiments, the antigens from HHV8 are selected from LANA-1 (ORF3); LANA-2 (vIRF3, K10.5); vCYC (ORF72); RTA (ORF50); vFLIP (ORF71); Kaposin (ORF12, K12); gB (ORF8); MIR1 (K3); SSB (ORF6); TS(ORF70), and a combination thereof.

In some embodiments, the clinically significant viruses can include but are not limited to HBV. In some embodiments, the viral antigens span immunogenic antigens from HBV. In some embodiments, the antigens from HBV are selected from (i) HBV core antigen, (ii) HBV Surface Antigen, and (iii) HBV core antigen and HBV Surface Antigen.

In embodiments, UVST compositions have specificity against any clinically significant or relevant viruses. For example, UVST compositions can comprise VSTs with specificity for viral antigens including antigens from HHV8 and/or HBV.

In some embodiments, the clinically significant viruses can include but are not limited to a coronavirus. In some embodiments, the coronavirus is a α-coronavirus (α-CoV). In some embodiments, the coronavirus is a β-coronavirus (β-CoV). In some embodiments, the β-CoV is selected from SARS-CoV, SARS-CoV2, MERS-CoV, HCoV-HKU1, and HCoV-OC43. In some embodiments, the coronavirus is SARS-CoV2. In some embodiments, the SARS-CoV2 antigen comprises one or more antigen selected from the group consisting of (i) nsp1; nsp3; nsp4; nsp5; nsp6; nsp10; nsp12; nsp13; nsp14; nsp15; and nsp16; (ii) Spike (S); Envelope protein (E); Matrix protein (M); and Nucleocapsid protein (N); and (iii) SARS-CoV-2 (AP3A); SARS-CoV-2 (NS7); SARS-CoV-2 (NS8); SARS-CoV-2 (ORF10); SARS-CoV-2 (ORF9B); and SARS-CoV-2 (Y14). In embodiments, UVST compositions can comprise VSTs with specificity for viral antigens including antigens from a coronavirus, e.g., SARS-CoV2.

In some embodiments, the antigen specific T cells in the compositions can be generated by contacting mononuclear cells (MNCs) with a plurality of pepmix libraries. In some embodiments, the antigen specific T cells in the compositions can be generated by contacting peripheral blood mononuclear cells (PBMCs) with a plurality of pepmix libraries. In some embodiments, each pepmix library contains a plurality of overlapping peptides spanning at least a portion of a viral antigen. In some embodiments, at least one of the plurality of pepmix libraries spans a first antigen from PIV. In some embodiments, at least one additional pepmix library of the plurality of pepmix libraries spans each second antigen.

In some embodiments, the antigen specific T cells can be generated by contacting T cells with dendritic cells (DCs) nucleofected with at least one DNA plasmid. In some embodiments, the DNA plasmid can encode the PIV antigen. In some embodiments, the at least one DNA plasmid encodes each second antigen. In some embodiments, the plasmid encodes at least one PIV antigen and at least one of the second antigens. In some embodiments, the compositions as described herein comprise CD4+ T-lymphocytes and CD8+ T-lymphocytes. In some embodiments, the compositions comprise antigen specific T cells expressing αβT cell receptors. In some embodiments, the compositions comprise MHC-restricted antigen specific T cells.

In some embodiments, the T cells can be cultured ex vivo in the presence of one or more cytokines selected from IL-1, IL-2, IL-4, IL-6, IL-7, IL-12, IL-15, IL17, IL18 and IL-21. In embodiments, the T cells can be cultured ex vivo in the presence of one or more cytokines selected from IL-1, IL-4, IL-6, IL-7, IL-12, IL-15, IL17, IL18 and IL-21. In embodiments, the T cells can be cultured ex vivo in the presence of one or more cytokines selected from IL-1, IL-4, IL-6, IL-7, IL-12, IL-15, IL17, IL18 and IL-21; wherein the cytokines do not comprise IL-2. In some embodiments, the antigen specific T cells can be cultured ex vivo in the presence of both IL-7 and IL-4. In some embodiments, the multivirus antigen specific T cells have expanded sufficiently within 9 days, 10 days, 11 days, 12 days, 13 days, 14 days, 15 days, 16 days, 17 days, 18 days, 19 days, 20 days inclusive of all ranges and subranges therebetween, of culture such that they are ready for administration to a patient. In some embodiments, the multivirus antigen specific T cells have expanded sufficiently within any number of days that are suitable for the compositions ad described herein.

The present disclosure provides compositions comprising antigen specific T cells that exhibit negligible alloreactivity. In some embodiments, the compositions comprising antigen specific T cells that exhibit less activation induced cell death of antigen-specific T cells harvested from a patient than corresponding antigen-specific T cells harvested from the same patient. In some embodiments, the compositions are not cultured in the presence of both IL-7 and IL-4. In some embodiments, the compositions comprising antigen specific T cells exhibit viability of greater than 70%.

In some embodiments, the compositions are negative for bacteria and fungi for at least 1 days, at least 2 days, at least 3 days, at least 4 days, at least 5 days, at least 6 days at least 7 days, at least 8 days, at least 9 days, at least 10 days, in culture. In some embodiments, the composition is negative for bacteria and fungi for at least 7 days in culture. In some embodiments, the compositions exhibit less than 1 EU/ml, less than 2 EU/ml, less than 3 EU/ml, less than 4 EU/ml, less than 5 EU/ml, less than 6 EU/ml, less than 7 EU/ml, less than 8 EU/ml, less than 9 EU/ml, less than 10 EU/ml of endotoxin. In some embodiments, the compositions exhibit less than 5 EU/ml of endotoxin. In some embodiments, the compositions are negative for mycoplasma.

In some embodiments, the pepmixes used for constructing the polyclonal population of antigen specific T cells are chemically synthesized. In some embodiments, the pepmixes are optionally >10%, >20%, >30%, >40%, >50%, >60%, >70%, >80%, >90%, inclusive of all ranges and subranges therebetween, pure. In some embodiments, the pepmixes are optionally >90% pure.

In some embodiments, the antigen specific T cells are Th1 polarized. In some embodiments, the antigen specific T cells are able to lyse viral antigen-expressing targets cells. In some embodiments, the antigen specific T cells are able to lyse other suitable types of antigen-expressing targets cells. In some embodiments, the antigen specific T cells in the compositions do not significantly lyse non-infected autologous target cells. In some embodiments, the antigen specific T cells in the compositions do not significantly lyse non-infected autologous allogenic target cells.

The present disclosure provides pharmaceutical compositions comprising any compositions formulated for intravenous delivery (e.g., a pharmaceutical composition comprising an antigen-specific T cell line from a donor minibank described herein formulated for intravenous delivery). In some embodiments, the compositions are negative for bacteria for at least 1, at least 2, at least 3, at least 4, at least 5, at least 6, at least 7, at least 8 days, at least 9 days, at least 10 days, in culture. In some embodiments, the compositions are negative for bacteria for at least 7 days in culture. In some embodiments, the compositions are negative for fungi for at least 1, at least 2, at least 3, at least 4, at least 5, at least 6, at least 7, at least 8 days, at least 9 days, at least 10 days, in culture. In some embodiments, the compositions are negative for fungi for at least 7 days in culture.

The present pharmaceutical compositions exhibit less than 1 EU/ml, less than 2 EU/ml, less than 3 EU/ml, less than 4 EU/ml, less than 5 EU/ml, less than 6 EU/ml, less than 7 EU/ml, less than 8 EU/ml, less than 9 EU/ml, or less than 10 EU/ml of endotoxin. In some embodiments, the present pharmaceutical compositions are negative for mycoplasma.

The present disclosure provides methods of lysing a target cell comprising contacting the target cell with the compositions or pharmaceutical compositions as described herein (e.g., an antigen-specific T cell line from a donor minibank described herein or a pharmaceutical composition comprising such a T cell line formulated for intravenous delivery). In some embodiments, the contacting between the target cell and the compositions or pharmaceutical compositions occurs in vivo in a subject. In some embodiments, the contacting between the target cell and the compositions or pharmaceutical compositions occurs in vivo via administration of the antigen specific T cells to a subject. In some embodiments, the subject is a human.

The present disclosure provides methods of treating or preventing a viral infection comprising administering to a subject in need thereof the compositions or the pharmaceutical compositions as described herein (e.g., an antigen-specific T cell line from a donor minibank described herein or a pharmaceutical composition comprising such a T cell line formulated for intravenous delivery). In some embodiments, the amount of antigen specific T cells that are administered range between 5×10³ and 5×10⁹ antigen specific T cells/m², 5×10⁴ and 5×10⁸ antigen specific T cells/m², 5×10⁵ and 5×10⁷ antigen specific T cells/m², 5×10⁴ and 5×10⁸ antigen specific T cells/m², 5×10⁶ and 5×10⁹ antigen specific T cells/m², inclusive of all ranges and subranges therebetween. In some embodiments, the antigen specific T cells are administered to the subject. In some embodiments, the subject is immunocompromised. In some embodiments, the subject has acute myeloid leukemia. In some embodiments, the subject has acute lymphoblastic leukemia. In some embodiments, the subject has chronic granulomatous disease.

In some embodiments, the subject can have one or more medical conditions. In some embodiments, the subject receives a matched related donor transplant with reduced intensity conditioning prior to receiving the antigen specific T cells. In some embodiments, the subject receives a matched unrelated donor transplant with myeloablative conditioning prior to receiving the antigen specific T cells. In some embodiments, the subject receives a haplo-identical transplant with reduced intensity conditioning prior to receiving the antigen specific T cells. In some embodiments, the subject receives a matched related donor transplant with myeloablative conditioning prior to receiving the antigen specific T cells. In some embodiments, the subject has received a solid organ transplantation. In some embodiments, the subject has received chemotherapy. In some embodiments, the subject has an HIV infection. In some embodiments, the subject has a genetic immunodeficiency. In some embodiments, the subject has received an allogeneic stem cell transplant. In some embodiments, the subject has more than one medical conditions as described in this paragraph. In some embodiments, the subject has all medical conditions as described in this paragraph.

In some embodiments, the composition as described herein is administered to the subject a plurality of times. In some embodiments, the composition as described herein is administered to the subject more than one time. In some embodiments, the composition as described herein is administered to the subject more than two times. In some embodiments, the composition as described herein is administered to the subject more than three times. In some embodiments, the composition as described herein is administered to the subject more than four times. In some embodiments, the composition as described herein is administered to the subject more than five times. In some embodiments, the composition as described herein is administered to the subject more than six times. In some embodiments, the composition as described herein is administered to the subject more than seven times. In some embodiments, the composition as described herein is administered to the subject more than eight times. In some embodiments, the composition as described herein is administered to the subject more than nine times. In some embodiments, the composition as described herein is administered to the subject more than ten times. In some embodiments, the composition as described herein is administered to the subject a number of times that are suitable for the subjects.

In some embodiments, the administration of the composition effectively treats or prevents a viral infection in the subject. In some embodiments, the viral infection is parainfluenza virus type 3. In some embodiments, the viral infection is respiratory syncytial virus. In some embodiments, the viral infection is Influenza. In some embodiments, the viral infection is human metapneumovirus.

The present disclosure provides compositions comprising a polyclonal population of antigen specific T cells that recognize a plurality of viral antigens, and donor minibanks as described herein containing a plurality of cell lines containing such antigen specific T cells. The present disclosure provides that the plurality of viral antigens comprise at least one antigen. In some embodiments, the at least one antigen can be parainfluenza virus type 3 (PIV). In some embodiments, the at least one antigen can be respiratory syncytial virus. In some embodiments, the at least one antigen can be Influenza. In some embodiments, the at least one antigen can be human metapneumovirus.

In some embodiments, the present disclosure provides a polyclonal population of antigen specific T cells that recognize a plurality of viral antigens comprising at least one antigen from each of parainfluenza virus type 3 (PIV) respiratory syncytial virus, Influenza, and human metapneumovirus, as well as donor minibanks as described herein containing a plurality of cell lines containing such antigen specific T cells. In some embodiments, the present disclosure provides a polyclonal population of antigen specific T cells that recognize a plurality of viral antigens comprising the plurality of viral antigens comprise at least two antigens from each of parainfluenza virus type 3 (PIV) respiratory syncytial virus, Influenza, and human metapneumovirus, as well as donor minibanks as described herein containing a plurality of cell lines containing such antigen specific T cells.

In some embodiments, the plurality of antigens comprise PIV antigen M, PIV antigen HN, PIV antigen N, PIV antigen F, influenza antigen NP1, influenza antigen MP1, RSV antigen N, RSV antigen F, hMPV antigen M, hMPV antigen M2-1, hMPV antigen F, and hMPV antigen N. In some embodiments, the plurality of antigens can be selected from any of PIV antigen M, PIV antigen HN, PIV antigen N, PIV antigen F, influenza antigen NP1, influenza antigen MP1, RSV antigen N, RSV antigen F, hMPV antigen M, hMPV antigen M2-1, hMPV antigen F, and hMPV antigen N.

In some embodiments, the present disclosure provides pharmaceutical compositions comprising the compositions as described herein formulated for intravenous delivery. In some embodiments, the composition as described herein is negative for bacteria. In some embodiments, the composition as described herein is negative for fungi. In some embodiments, the composition as described herein is negative for bacteria or fungi for at least 1 days, at least 2 days, at least 3 days, at least 4 days, at least 5 days, at least 6 days, at least 7 days, at least 8 days, at least 9 days, at least 10 days, in culture. In some embodiments, the composition as described herein is negative for bacteria or fungi for at least 7 days in culture.

In some embodiments, the pharmaceutical compositions formulated for intravenous delivery exhibit less than 1 EU/ml, less than 2 EU/ml, less than 3 EU/ml, less than 4 EU/ml, less than 5 EU/ml, less than 6 EU/ml, less than 7 EU/ml, less than 8 EU/ml, less than 9 EU/ml, or less than 10 EU/ml of endotoxin. In some embodiments, the pharmaceutical compositions formulated for intravenous delivery are negative for mycoplasma.

### EXAMPLES

### Example 1. Construction of a Donor Bank of CMV-Specific VST (CMVST)

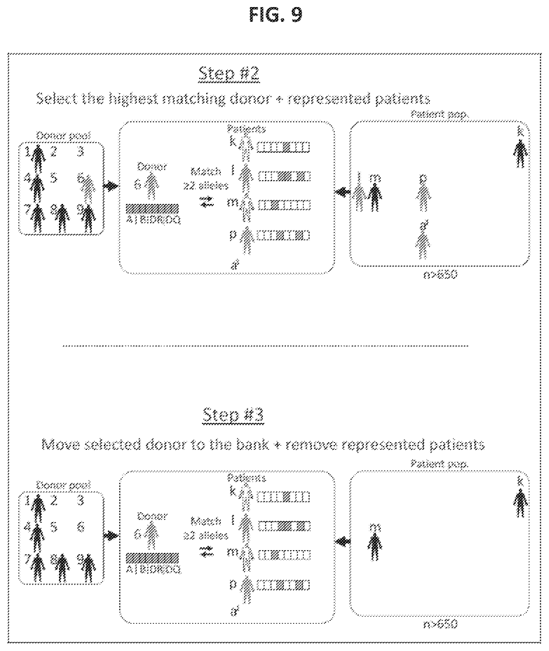

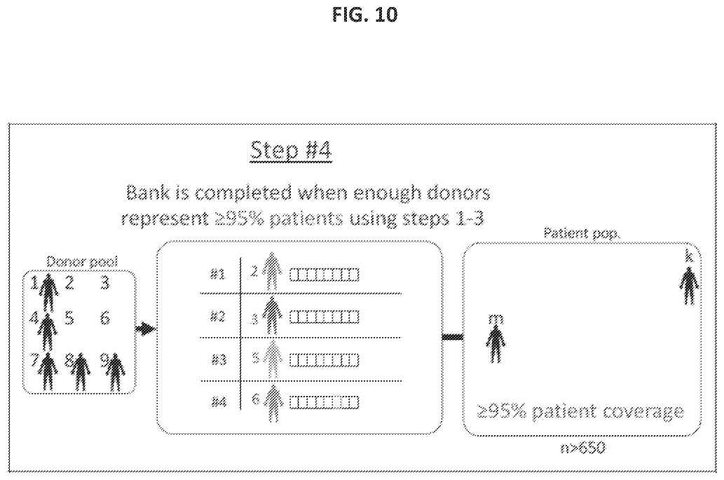

Selection of donors for CMVST generation: To ensure that a clinically effective line could be provided for the majority of the allogeneic HSCT patient population, we developed a donor selection algorithm to choose the best possible donors from a given donor pool for producing cell therapy products for a given patient population. The HLA types of 666 allogeneic HSCT recipients treated in the Houston region (Houston Methodist or Texas Children's Hospital) were analyzed, which has a diverse ethnic make-up that is similar to the United States as a whole. These HSCT recipient HLAs were then compared with the HLA types of a pool of diverse, healthy, eligible CMV seropositive donors. In an initial step (FIG. 2, Step #1) the HLA type of each of the healthy donors in the general donor pool was individually compared with the HLA type of each of the patients in the patient pool, and the highest matching donor (also referred to herein as the “greatest matched donor”) was identified as being the donor that matched on at least 2 HLA alleles with the greatest number of patients in the patient pool (FIG. 2, Step #2). This donor was removed from the general donor pool and all patients accommodated by this donor (i.e., matched on at least 2 HLA alleles to that donor) were also removed from the other unmatched patients in the patient population; thus leading to a donor pool depleted by one donor and an unmatched patient population depleted by the number of patients matched to the first donor on 2 or more HLA alleles (FIG. 2, Step #3). Subsequently these steps were repeated a second, third, etc. time, each time identifying the donor in the remaining donor pool who matched on at least 2 HLA alleles with the greatest number of patients that were at that time remaining in unmatched patient population and then removing both that donor and all those patients matched to that donor from further consideration (FIGS. 4-9), until a panel of donors was generated that covered (i.e., were matched on at least 2 HLA alleles with) at least 95% of the patients in the analyzed patient population (FIG. 10). That first panel of donors was set aside for use in constructing a first minibank of virus specific T cells.

At this point, all of the patients that had been removed from the_unmatched patient population in the construction of the first donor minibank were reintroduced into the patient population (but none of the previously removed donors were reintroduced into the general donor population), and this procedure was then repeated a second time to identify a second panel of donors that covered (i.e., were matched on at least 2 HLA alleles with) at least 95% of the patients in the analyzed patient population to create a second donor minibank. This ensured that patients would have more than one potential well-matched donor option in discreate donor minibanks. Using this model, it was found that only 8 well-selected donors would provide >95% of the patient population with a T cell product that was matched on at least 2 HLA antigens; and in this case, further increasing the donor pool did not significantly increase the number of matches. Eight of these donors were then selected with the goal to provide coverage suitable CMVST line (?2 shared HLA antigens) with confirmed CMV activity to ≥95% of this diverse population of allogeneic HSCT recipients.

Third-party CMVST bank preparation: All donors gave written informed consent on an IRB approved protocol and met blood bank eligibility criteria. For manufacturing, a unit of blood was collected by peripheral blood draw and PBMCs isolated by ficoll gradient. 10×10⁶ PBMCs were seeded in a G-Rex 5 bioreactor (Wilson Wolf, Minneapolis, MN) and cultured in T cell media [Advanced RPMI 1640 (HyClone Laboratories Inc. Logan, Utah), 45% Click's (Irvine Scientific, Santa Ana, CA), 2 mM GlutaMAX™] TM-I (Life Technologies Grand Island, NY), and 10% Fetal Bovine Serum (Hyclone)] containing 800 U/ml IL4 and 20 ng/ml IL7 (R&D Systems, Minneapolis, MN) and IE1, pp65 pepmixes (2 ng/peptide/ml) (JPT Peptide Technologies Berlin, Germany). On day 9-12 post initiation, T cells were harvested, counted and restimulated with autologous pepmix-pulsed irradiated PBMCs [1:4 effector: target (E:T)—4×10⁵ CMVSTs: 1.6×10⁶ irradiated PBMCs/cm2] with IL4 (800 U/ml) and IL7 (20 ng/ml) in a G-Rex-100M. On day 3-4 of culture, the cells were fed with 200 ng/ml IL2 (Prometheus Laboratories, San Diego, CA), and 9-12 days post second stimulation, T cells were harvested for cryopreservation. At the time of cryopreservation, each line was microbiologically tested, immunophenotyped [CD3, CD4, CD8, CD14, CD16, CD19, CD25, CD27, CD28, CD45, CD45RA, CD56, CD62L CD69, CD83, HLA DR and 7AAD (Becton Dickinson, Franklin Lakes, NJ)], and evaluated for virus specificity by IFNγ enzyme-linked immunospot (ELISpot) assay. A cell line was defined as “reactive” when the frequency of reactive cells, as measured by IFNγ ELISpot assay, was >30 spot-forming cells (SFC)/2×10⁵ input viral specific T cells.

Clinical trial design: This was a single center Phase I study (NCT02313857) conducted under an IND from the Food and Drug Administration (FDA) and approved by the Baylor College of Medicine Institutional Review Board (IRB). The study was open to allogeneic HSCT recipients with CMV infections or disease that had persisted for at least 7 days despite standard therapy defined as treatment with ganciclovir, foscarnet, or cidofovir. Exclusion criteria included treatment with prednisone (or equivalent) ≥0.5 mg/kg, respiratory failure with oxygen saturation of <90% on room air, other uncontrolled infections, and active GVHD ≥grade II. Patients who received ATG, Campath, other T cell immunosuppressive monoclonal antibodies, or a donor lymphocyte infusion (DLI) within 28 days of the proposed administration date were also excluded from participation. Patients initially gave their consent to search for a suitable VST line (with ≥2 shared HLA antigens), and if available and if patients met eligibility criteria, they could be enrolled on the treatment portion of the study. Each patient received a single intravenous infusion of 2×10⁷ partially HLA-matched VSTs/m2 with the option to receive a second infusion after 4 weeks and additional infusions at bi-weekly intervals thereafter. Therapy with standard antiviral medications could be administered at the discretion of the treating physician.

Safety endpoints: The primary objective of this pilot study was to determine the safety of CMVSTs in HSCT recipients with persistent CMV infections/disease. Toxicities were graded by the NCI Common Terminology Criteria for Adverse Events (CTCAE), Version 4.X. Safety endpoints included acute GvHD grades III-IV within 42 days of the last CMVST dose, infusion-related toxicities within 24 hours of infusion or grades 3-5 non-hematologic adverse events related to the T cell product within 28 days of the last CMVST dose and not attributable to a pre-existing infection, the original malignancy or pre-existing co-morbidities. Acute and chronic GVHD, if present, were graded according to standard clinical definitions.1,2 The study was monitored by the Dan L. Duncan Cancer Center Data Review Committee.

Assessment of outcomes: CMV loads in peripheral blood were monitored by quantitative PCR (qPCR) in Clinical Laboratory Improvement Amendments (CLIA)-approved laboratories. A complete response (CR) of the virus to treatment was defined as a decrease in viral load to below the threshold of detection by qPCR and resolution of clinical signs and symptoms of tissue disease (if present at baseline). A partial response (PR) was defined as a decrease in viral load of at least 50% from baseline. Clinical and virological responses were assigned at week 6 post CMVST infusion.

Immune Monitoring: ELISpot analysis was used to determine the frequency of circulating T cells that secreted IFNγ in response to CMV antigens and peptides. Clinical samples were collected prior to and at weeks 1, 2, 3, 4, 6 and 12 post-infusion. As a positive control, PBMCs were stimulated with Staphylococcal Enterotoxin B (1 μg/ml) (Sigma-Aldrich Corporation, St Louis, MO). IE1 and pp65 pepmixes (JPT Technologies, Berlin, Germany), diluted to 1000 ng/peptide/ml, were used to track donor-derived CMVSTs post-infusion. When available, peptides representing known epitopes (Genemed Synthesis Inc., San Antonio, TX diluted to 1250 ng/ml) were also used in ELISpot assays. For ELISpot analyses, PBMCs were resuspended at 5×10⁶/ml in T cell medium and plated in 96 well ELISpot plates. Each condition was run in duplicate. After 20 hours of incubation, plates were developed as previously described, dried overnight at room temperature in the dark, and then sent to Zellnet Consulting (New York, NY) for quantification. Interferon-γ spot-forming cells (SFC) and input cell numbers were plotted, and the frequency of T cells specific for each antigen was expressed as specific SFC per input cell numbers.

Statistical Analysis: Descriptive statistics were calculated to summarize data. Antiviral responses were summarized, and the response rate was estimated along with exact 95% binomial confidence intervals. Viral load and T cell frequency data were plotted to graphically illustrate the patterns of immune responses over time. Comparisons of changes in viral load and T cell frequency pre- and post-infusion were performed using Wilcoxon signed-ranks test. Data were analyzed with SAS system (Cary, NC) version 9.4 and R version 3.2.1. P-values <0.05 were considered statistically significant.

Results

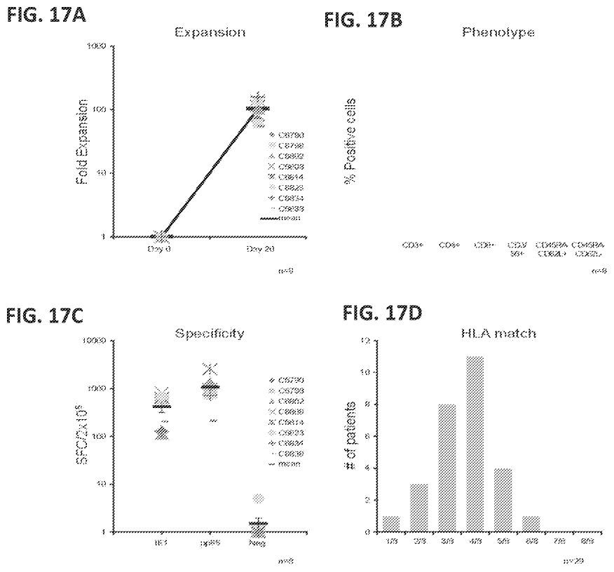

Third party CMVST bank: A bank of CMVSTs was generated from 8 CMV seropositive donors chosen to represent the diverse HLA profile of the transplant population (Table 1). A median of 7.7×10⁸ PBMCs (range 4.6-8.8×10⁸) were isolated from a single blood draw (median of 425 ml). To expand CMVSTs, PBMCs were exposed to pepmixes spanning pp65 and IE1 and over 20 days in culture a mean fold expansion of 102±12 (FIG. 17A) was achieved. The resulting cells were almost exclusively CD3+(99.3±0.4%), comprising both CD4+(21.3±7.5%) and CD8+(74.7±7.8%) subsets that expressed central CD45RA−/62L+(58.5±4.8%) and effector CD45RA−/62L− (35.3±4.6%) memory markers (FIG. 17B). All 8 lines were reactive against the stimulating CMV antigens (IE1 419±100 SFC/2×10⁵ and pp65 1069±230, FIG. 17C). Table 1 summarizes the characteristics of the cell lines. Of these 8 lines, 6 products were administered to 10 treated study patients.

Screening: 29 allogeneic HSCT recipients with CMV infections were referred by their primary BMT providers for study participation, and from a bank of 8 lines, a suitable product (minimum 2/8 HLA match threshold) was identified for infusion in 28 cases (96.6%; 95% CI: 82.2%-99.9%). A 2/8 HLA match threshold was established based on retrospective analysis performed on previous third party study which demonstrated clinical benefit associated with the administration of such HLA-matched products. HLA class I or class II matching did not appear to influence outcome. Of note, on the current study, most products were matched at ≥4 antigens (FIG. 18D). Of the 28 patients with available lines, 17 patients did not receive cells because they responded to standard antiviral treatment and one patient was ineligible due to a recent DLI.

Characteristics of treated patients: The characteristics of the 10 patients (pediatric n=7 and adults n=3) treated for persistent infections are summarized in Table 2 and included 2 African-American recipients, 3 patients of white Hispanic origin and 5 non-Hispanic Caucasian recipients. Three of the 10 patients had confirmed virus-associated disease [CMV retinitis (n=1), diarrhea attributed to CMV colitis (n=2)]. CMVSTs (matching at 2-6/8 HLA antigens) were administered between days 46 and 365 (median day 133) post-transplant. Seven patients had infections that were refractory to standard antiviral treatment for a median of 24 days (mean of 48 days; range 14 to 211 days), and 3 of the patients harbored viruses with mutations that conferred resistance to conventional antivirals. Prior to immunotherapeutic intervention, 6 of these patients had experienced severe adverse events (SAEs) associated with conventional antivirals that included acute kidney injury (n=4), foscarnet-induced renal tubulopathy (n=1) and severe foscarnet-associated pancreatitis (n=1), which in 3 cases precluded further treatment with any conventional drugs.

Clinical safety: All infusions were well tolerated. Except for one patient who developed a transient isolated fever 8 hours after infusion, no immediate toxicities were observed. One patient developed a mild maculopapular rash on his trunk, which appeared suggestive of a viral exanthem and spontaneously resolved within a few days without topical or systemic treatment. No cases of cytokine release syndrome (CRS) or other toxicities related to the infused CMVSTs were observed, and none of the patients developed graft failure, autoimmune hemolytic anemia or transplant associated microangiopathy. Patients were followed for 6 weeks for acute GvHD and 12 months for chronic GvHD. Despite the HLA disparity between the patients and the infused cells, none of the patients developed recurrent or de novo acute or chronic GvHD post treatment (Table 3), including 3 patients with a prior history [grade II (n=2) or III (n=1)] of acute GvHD.

Clinical Responses: All 10 infused patients responded to CMVSTs with 7 CRs and 3 PRs, for a cumulative response rate of 100% (95% CI: 69.2-100.0%) by week 6. The average plasma viral load reduction at week 6 was 89.8% (+/−21.4%). FIG. 18 summarizes the virological outcomes of all treated patients based on sequential viral load measurements. Of note, clinical benefit was achieved not only in patients with refractory infections, but also in the 3 individuals with tissue disease [CMV retinitis (n=1), diarrhea attributed to CMV colitis (n=2)].

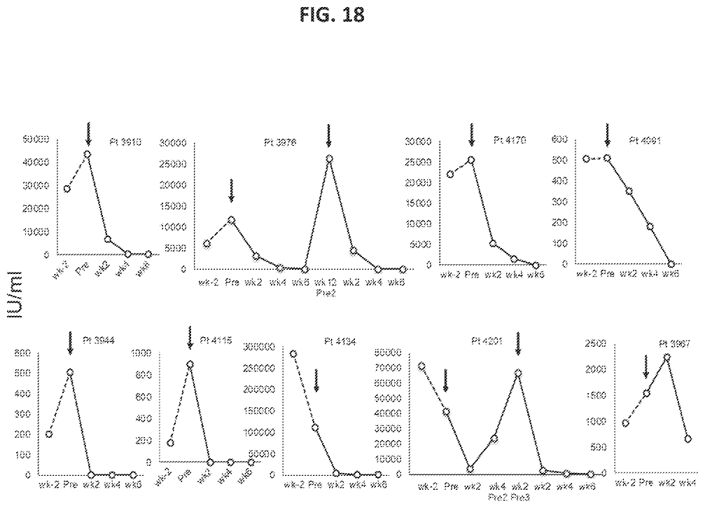

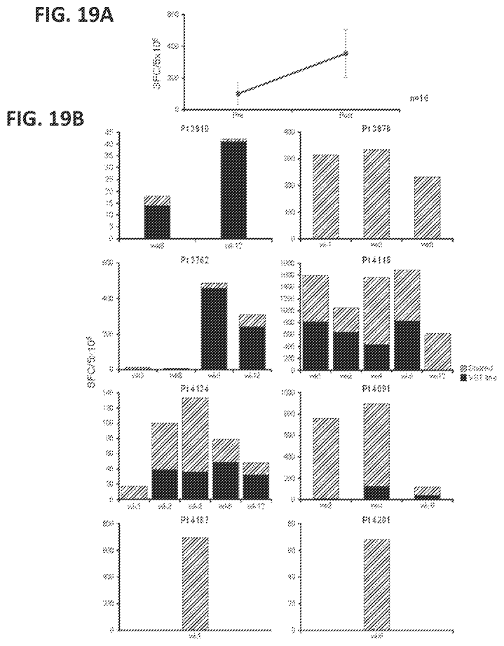

Eight patients received a single infusion of CMVSTs, 1 patient (3976) had 2 infusions and 1 (4201) had 3 infusions of CMVSTs. Patient 3976 had a CR at week 6, but relapsed with increasing virus loads at week 10. Eighty days after the first infusion, he received a second infusion with the same CMVST line that resulted in a sustained CR. Patient 4201 received a second infusion of the same CMVSTs 28 days after the initial administration but failed to respond and hence, 2 weeks later was administered a third infusion with a different CMVST line and achieved a sustained CR. The clinical and virological responses achieved in these patients were associated with an increase in virus-reactive CMVSTs in 8 of the 10 treated patients [increase from mean 126±84 SFC pre-infusion to peak of 443±178 per 5×10⁵ PBMCs (p=0.023; FIG. 19A)].

T cell persistence: To evaluate if the CMVST infusions contributed to the protective effects seen in these patients and to evaluate the in vivo longevity of these partially HLA-matched VSTs, the specificity of CMVSTs were examined in patient PBMCs before and after infusion using HLA-restricted epitope peptides restricted to the line infused. Functional T cells of confirmed third-party origin were detected in 5 patients for whom HLA-restricting peptide reagents were available, which persisted for up to 12 weeks; in all 8 patients antiviral responses restricted by the HLA alleles shared between the patient and the CMVST line (FIG. 19B) were observed. Thus, it was inferred that the infused CMVSTs induced an antiviral effect resulting in the control of CMV infections.

In the Phase I trial, third party CMVSTs were administered to treat CMV infections/disease in allogeneic HSCT recipients who had failed at least 14 day of treatment with ganciclovir and/or foscarnet or could not tolerate standard antiviral medications. Notable exclusion criteria were patients with active GvHD or receiving corticosteroids at moderate or high doses. A bank of CMVSTs was generated from just 8 healthy donors, which were carefully selected based on their HLA profile to provide broad coverage to a racially and ethnically diverse allogeneic HSCT patient population. Indeed, of the 29 patients screened for study participation, a suitable line (minimum 2 shared HLA antigen threshold) for 28 (96.6%; 95% CI: 82.2-99.9%) was identified, attesting to the feasibility of providing broad patient coverage with a small, well-chosen cell bank. Of these 28 patients, 10 from diverse backgrounds (2 African-American, 3 of white Hispanic origin and 5 non-Hispanic Caucasian) were treated and all achieved virological and clinical benefit, without experiencing acute or chronic GvHD or other toxicities. This was notable, since 6 had previously experienced serious adverse events including acute kidney injury, renal tubulopathy and pancreatitis, related to conventional antivirals. This Phase I trial showcases the safety and clinical benefit associated with the administration of 3^{rd} party virus-reactive T cells, sourced from a small and carefully designed donor bank, for the treatment of refractory CMV infections.

Despite decreasing rates of disease in recent decades, CMV remains a major cause of morbidity after allogeneic HSCT; in a recent CIBMTR report where data from 9469 patients [transplanted from 2003-2010 for AML (n=5310), ALL (n=1883), CML (n=1079) and MDS (n=1197)] was interrogated and CMV reactivation was associated with higher non-relapse mortality as well as lower overall survival among all 4 disease categories. Furthermore, in a recent study of 208 patients transplanted between 2008-2013, the average length of in-hospital stay was found to be prolonged by 26 days in patients with CMV reactivation, while the occurrence of more than one CMV reactivation episode increased the costs of an allogeneic HSCT by 25-30% (p<0.0001), highlighting the economic burden of CMV management.

Foscarnet and ganciclovir are frequently used to treat CMV infections after HSCT. However, outside of ganciclovir for CMV retinitis, their use is off-label, and both drugs are associated with significant side effects, particularly renal disease and graft suppression. When used prophylactically, letermovir, a cytomegalovirus DNA terminase complex inhibitor, decreased the incidence of CMV infection/reactivation post-transplant6, and since FDA approval (for CMV prophylaxis in adult HSCT patients) in 2017, is increasingly used in high-risk patients. However the CMV Resistance Working Group of the multidisciplinary CMV Drug Development Forum expects that the wider prophylactic use of letermovir will increase the emergence of organisms that are resistant to conventional antivirals if a CMV breakthrough infection does occur. Indeed, letermovir-resistant CMV strains are increasingly reported and clinical outcomes in those with resistant disease are poor and associated with progressive tissue disease and mortality.

CMVSTs provide an alternative strategy to target both initial reactivations as well as drug-resistant viral strains, as previously reported by our group and others. Indeed 30% of the patients treated with CMVSTs in the current study were infected with viral strains confirmed to be resistant to one or more conventional antiviral drugs.

One goal of the current study was to prepare a CMV-specific T cell bank with sufficient diversity to cover the majority of allogeneic HSCT recipients referred for treatment. Thus, the HLA types of >600 allogeneic HSCT recipients were prospectively compared with a pool of diverse healthy, eligible (CMV seropositive) donors from whom CMVSTs could be generated to identify the minimum cohort that would provide the patients with well-matched products. Using this model it was found that only 8 well-selected donors would provide >95% of the patient population with a T cell product that was matched on at least 2 HLA antigens; further increasing the donor pool would not significantly increase the number of matches. The current study, in which a suitable line was identified for 28 of 29 patients (96.5%) screened for clinical participation, supports the theory that such a donor bank could effectively supply the majority of the allogeneic HSCT patient population.

The racial and ethnic diversity was compared within the transplant patient population with that of the U.S. transplant population (Table 4). This revealed that the diversity within our patient population was similar if not slightly more diverse than the U.S. population. This suggests that the small cell bank developed for the current study could be broadly applied for clinical use across the country. Universal use of the tested CMVSTs across transplant centers is made more feasible by the ability to produce sufficient material to generate cells for >2,000 infusions from a single donor collection. Thus, one could envisage a decentralized distribution model of “off the shelf” third party virus-reactive T cells, ensuring on-demand availability of cells.

In summary, the data indicate that a well characterized bank of CMV-reactive T cells prepared from just 8 well-chosen third party donors can supply the majority of patients with refractory CMV infections with an appropriately matched line that can provide safe and effective antiviral activity.

TABLE 1
Characteristics of generated VST lines.

| | CMV | CMV | | | | | | | | | | | | |
|---|---|---|---|---|---|---|---|---|---|---|---|---|---|---|
| | Speci- | Speci- | | | | | | | | | | | | |
| | ficity | ficity | | | | | | | | | | | | |
| VST | SFC/ | SFC/ | | | | | CD45RO+/ | CD45RO+/ | | | | | # of | # of |
| line | 1 × 10⁵ | 1 × 10⁵ | CD3 | CD4 | CD8 | CD56 | CD62L+ | CD62L− | | | | | patients | patients |
| (C#) | IE1 | pp65 | (%) | (%) | (%) | (%) | (%) | (%) | HLA-A | HLA-B | HLA-DR | HLA-DQ | Screened* | treated |
| 6790 | 127 | 1186 | 97.81 | 74.23 | 19.48 | 3.88 | 75.45 | 16.33 | 02.33 | 15.44 | 07.13 | 02.06 | 4 | 3 |
| 6798 | 612 | 805 | 98.79 | 17.75 | 75.73 | 4.05 | 40.3 | 44.83 | 02.02 | 40.52 | 04.08 | 03.03 | 6 | 4 |
| 6802 | 113 | 1354 | 99.66 | 5.20 | 92.82 | 1.69 | 69.75 | 27.51 | 11.23 | 35.57 | 01.07 | 03.05 | 1 | 0 |
| 6808 | 827 | 986 | 99.77 | 12.59 | 83.18 | 3.10 | 74.09 | 20.13 | 02.24 | 40.52 | 04.13 | 03.06 | 4 | 1 |
| 6814 | 639 | 2573 | 99.68 | 28.25 | 69.85 | 0.99 | 41.56 | 55.78 | 2.24 | 8.14 | 01.03 | 02.05 | 1 | 1 |
| 6823 | 700 | 717 | 99.39 | 10.99 | 86.49 | 1.51 | 47.59 | 48.59 | 11.68 | 07.35 | 03.07 | 02.02 | 3 | 1 |
| 6834 | 128 | 725 | 99.77 | 15.40 | 82.90 | 2.27 | 64.64 | 32.72 | 02.24 | 15.35 | 04.09 | 03.03 | 6 | 1 |
| 6838 | 205.5 | 211 | 99.75 | 5.57 | 87.46 | 8.76 | 54.50 | 36.42 | 02.30 | 13.35 | 07.08 | 02.06 | 1 | 0 |

SFC = spot forming cells;
*= indicates how frequently the VST lines was determined to be the most suitable line for a screened patient.

TABLE 2
Patient characteristics

| Patient | | | | | Type of | R/D CMV | # of | Days post- |
|---|---|---|---|---|---|---|---|---|
| ID# | Age | Ethnicity | Race | Diagnosis | transplant | serostatus | Infusions | transplant |
| 3910 | 12 | Non-Hispanic | African | Sickle Cell | MRD | Neg/Pos | 1 | 61 |
| | | | American | Anemia | | | | |
| 3944 | 45 | Hispanic | White | AML | UCB | Pos/Neg | 1 | 197 |
| 3976 | 13 | Hispanic | White | ALL | MUD | Pos/Pos | 2 | 46 |
| 3762 | 10 | Hispanic | White | HLH | MMUD | Pos/Neg | 1 | 161 |
| 3967 | 51 | Non-Hispanic | White | AML | UCB | Pos/Neg | 1 | 365 |
| 4091 | 70 | Non-Hispanic | White | CTCL | Haplo | Pos/Pos | 1 | 215 |
| 4115 | 3 | Non-Hispanic | White | Fanconi | MUD | Pos/Pos | 1 | 105 |
| | | | | Anemia | | | | |
| 4170 | 3 | Non-Hispanic | African | Sickle Cell | MRD | Neg/Pos | 1 | 76 |
| | | | American | Anemia | | | | |
| 4134 | 16 | Non-Hispanic | White | SCID | MUD | Pos/Pos | 1 | 218 |
| 4201 | 11 | Non-Hispanic | White | Anaplastic | MUD | Pos/Neg | 3 | 70 |
| | | | | Large cell | | | | |
| | | | | lymphoma | | | | |

AML: Acute myeloid leukemia, ALL: Acute lymphoblastic leukemia, HLH: Hemophagocytic Lymphohistiocytosis, CTCL: Cutaneous T-cell lymphoma, SCID: Severe combined immunodeficiency, MRD: Matched related donor, UCB: umbilical cord blood, MUD: Matched unrelated donor, MMUD: mismatched unrelated donor, Haplo: Haploidentical, R/D: Recipient/Donor, AKI: Acute kidney injury, CR: Complete response, PR: Partial response, AdV: Adenovirus.

TABLE 3
GvHD pre and post infusion

| Patient | Prior | | GvHD Rx/PPx | | |
|---|---|---|---|---|---|
| ID # | GvHD | Baseline | at infusion | aGvHD | cGvHD |
| 3910 | None | None | Cyclosporine | None | None |
| 3944 | None | None | Tacrolimus | None | None |
| 3976 | None | None | Tacrolimus | None | None |
| 3762 | None | None | None | None | None |
| 3967 | GI Grade | None | Sirolimus | None | None |
| | II | | | | |
| 4091 | GI, skin | None | Tacrolimus | None | None |
| | Grade II | | | | |
| 4115 | None | None | None | None | None |
| 4170 | None | None | Tacrolimus | None | None |
| 4134 | GI Grade | None | None | None | None |
| | III | | | | |
| 4201 | None | None | Tacrolimus | None | None |

aGvHD: acute Graft versus Host Disease, cGvHD: chronic Graft versus Host Disease, GI: Gastrointestinal, Rx: Treatment, PPx: Prophylaxis.

TABLE 4
Racial diversity of allogeneic HSCT recipients. A total
of 174 Program transplant centers are represented in the
US analysis. Each of these centers performed at least one
unrelated or related donor transplant over the three-year
window of time from Jan. 1, 2013, to Dec. 31, 2015.

| | US | | Baylor CCGT | |
|---|---|---|---|---|
| | (2013-2015) | | (2014-2018) | |
| Patient Race | Number (%) | | Number (%) | |
| White | 19,600 | (82%) | 608 | (74.8%) |
| Black or African American | 2,162 | (9%) | 141 | (17.3%) |
| Asian | 1,022 | (4%) | 49 | (6.0%) |
| Pacific Islander | 65 | (<1%) | 2 | (<1%) |
| American Indian or Alaskan | 133 | (1%) | 10 | (1.2%) |
| Native | | | | |
| Multiple Race^{a} | 160 | (1%) | n/a | |
| Unknown | 704 | (3%) | n/a | |
| Total | 23,846 | (100%) | 810 | (100%) |

### Example 2. Generation of Multivirus-Specific T Lymphocytes for the Prevention and Treatment of Respiratory Viral Infections

Our group has previously demonstrated that the adoptive transfer of in vitro expanded virus specific T cells (VSTs) can safely and effectively prevent and treat infections associated with both latent [Epstein-Barr virus (EBV), cytomegalovirus (CMV), BK virus (BKV), human herpesvirus 6 (HHV6) and lytic [adenovirus (AdV)] viruses in allogeneic HSCT recipients. Given that susceptibility to CARVs is associated with underlying cellular immune deficiency, in the current study we explored the feasibility of extending the therapeutic range of VST therapy to include Influenza, RSV, hMPV and PIV.

We described a mechanism by which a single preparation of polyclonal (CD4+ and CD8+) VSTs with specificity for 12 immunodominant antigens derived from our 4 target viruses can be rapidly generated using GMP-compliant manufacturing methodologies. The viral proteins used for stimulation were chosen on the basis of both their immunogenicity to T cells and their sequence conservation. The expanded cells are Th1-polarized, polyfunctional and selectively able to react to and kill, viral antigen-expressing target cells with no activity against non-infected autologous or allogeneic targets, attesting to both their selectivity for viral targets and their safety for clinical use.

In this study, the inventors exposed PBMCs from healthy donors to a cocktail of pepmixes (overlapping peptide libraries) spanning immunogenic antigens from certain target viruses [Influenza—NP1 and MP1; RSV—N and F; hMPV—F, N, M2-1 and M; PIV—M, HN, N and F] followed by expansion in the presence of activating cytokines in a G-Rex. Over 10-13 days the inventors achieved an average 8.5 fold expansion (increase from 0.25×10⁷ PBMCs/cm2 to mean 1.9±0.2×10⁷ cells/cm²; n=12).

In brief, PBMCs were obtained from healthy volunteers and HSCT recipients with informed consent using Baylor College of Medicine IRB-approved protocols (H-7634, H-7666) and were used to generate phytohemagglutinin (PHA) blasts and multi-R-VSTs. PHA blasts were generated as previously reported and cultured in VST medium [45% RPMI 1640 (HyClone Laboratories, Logan, Utah), 45% Click's medium (Irvine Scientific, Santa Ana, California), 2 mM GlutaMAX™-I (Life Technologies, Grand Island, New York), and 10% human AB serum (Valley Biomedical, Winchester, Virginia)] supplemented with interleukin 2 (IL2) (100 U/mL; NIH, Bethesda, Maryland), which was replenished every 2 days.

For generating multi-R-VST, pepmixes were generated. In brief, PBMCs were stimulated with peptide libraries (15mers overlapping by 11 aa) spanning Influenza A (NP1, MP1), RSV (N, F), hMPV (F, N, M2-1, M) (JPT Peptide Technologies, Berlin, Germany) and PIV antigens (M, HN, N, F) (Genemed Synthesis, San Antonio, TX). Lyophilized pepmixes were reconstituted in Dimethyl sulfoxide (DMSO) (Sigma-Aldrich) and stored at −80° C. For generating multi-R-VSTs, PBMCs (2.5×10⁷) were transferred to a G-Rex10 (Wilson Wolf Manufacturing Corporation, St. Paul, MN) with 100 ml of VST medium supplemented with IL7 (20 ng/ml), IL4 (800 U/ml) (R&D Systems, Minneapolis, MN) and pepmixes (2 ng/peptide/ml) and cultured for 10-13 days at 37° C., 5% CO₂.

Flow cytometry was then conducted for Multi-R-VSTs were surface-stained with monoclonal antibodies to: CD3, CD25, CD28, CD45RO, CD279 (PD-1) [Becton Dickinson (BO), Franklin Lakes, NJ], CD4, CD8, CD16, CD62L, CD69 (Beckman Coulter, Brea, CA) and CD366 (TIM-3) (Biolegend, San Diego, CA). Cells were acquired on a Gallios™ Flow Cytometer and analyzed with Kaluza® Flow Analysis Software (Beckman Coulter). Specifically, cells were pelleted in phosphate-buffered saline (PBS) (Sigma-Aldrich), then antibodies added in saturating amounts (5 μl) followed by incubation for 15 mins at 4° C. Subsequently, cells were washed, resuspended in 300 μl of PBS and at least 20,000 live cells acquired on a Gallios™ Flow Cytometer and analyzed with Kaluza® Flow Analysis Software (Beckman Coulter).

For intracellular cytokine staining, multi-R-VSTs were harvested, resuspended in VST medium (2×10⁶/ml) and 200 μL added per well of a 96-well plate. Cells were incubated overnight with 200 ng of individual test or control pepmixes along with Brefeldin A (1 μg/ml), monensin (1 μg/ml), CD28 and CD49d (1 μg/ml) (BD). Next, VSTs were washed with PBS, pelleted, surface-stained with CD8 and CD3 (5 μl/antibody/tube) for 15 mins at 4° C., then washed, pelleted, fixed and permeabilized with Cytofix/Cytoperm solution (BD) for 20 mins at 4° C. in the dark. After washing with Perm/Wash Buffer (BD), cells were incubated with 10 μL of IFNγ and TNFα antibodies (BD) for 30 minutes at 4° C. in the dark. Cells were then washed twice with Perm/Wash Buffer and at least 50,000 live cells were acquired on a Gallios™ Flow Cytometer and analyzed with Kaluza® Flow Analysis Software.

FoxP3 staining was performed using the eBioscience FoxP3 kit (Thermo Fisher Scientific, Waltham, MA), per manufacturers' instructions. Briefly, 1×10⁶ cells were surface-stained with CD3, CD4 and CD25 antibodies, then washed, resuspended in 1 ml fixation/permeabilization buffer and incubated for 1 hour at 4° C. in the dark. After washing with PBS, cells were resuspended in permeabilization buffer, incubated with 5 μL isotype or FoxP3 antibody (Clone PCH101) for 30 minutes at 4° C., then washed and acquired on a Gallios™ Flow Cytometer followed by analysis with Kaluza® Flow Analysis Software. Enzyme-linked immunospot (ELIspot) spot analysis was used to quantitate the frequency of IFNγ and Granzyme B-secreting cells. Briefly, PBMCs, magnetically selected T cell sub-populations and multi-R-VSTs were resuspended at 5×10⁶ or 2×10⁶ cells/ml in VST medium and 100p1 of cells was added to each ELIspot well. Cell selection was performed using magnetic beads and IS separation columns (Miltenyi Biotec, GmbH), according to manufacturer's instructions. Antigen-specific activity was measured after direct stimulation (500 ng/peptide/ml) with the individual stimulating [NP1, MP1 (Influenza); N, F (RSV); F, N, M2-1, M (hMPV); M, HN, N, F (PIV)], or control pepmixes (Survivin, WT1). Staphylococcal Enterotoxin B (SEB) (1 μg/ml) and PHA (1 μg/ml) were used as positive controls for PBMCs and VSTs, respectively. After 20 hours of incubation, plates were developed as previously described, dried overnight at room temperature and then sent to Zellnet Consulting (New York) for quantification. Spot-forming cells (SFC) and input cell numbers were plotted and the specificity threshold for VSTs was defined as ≥30 SFC/2×10⁵ input cells.

The multi-R-VST cytokine profile was evaluated using the MILLIPLEX High Sensitivity Human Cytokine Panel (Millipore, Billerica, MA). 2×10⁵ VSTs were stimulated with pepmixes (NP1, MP1, N, F, F, N, M2-1, M, M, HN, N, and F) (1 μg/ml) overnight. Subsequently, supernatant was collected, plated in duplicate wells, incubated overnight at 4° C. with antibody-immobilized beads, then washed and plated for 1 hour at room temperature with biotinylated detection antibodies. Finally, streptavidin-phycoerythrin was added for 30 minutes at room temperature. Samples were washed and analyzed on a Luminex 200 (XMAP Technology) using the xPONENT software.

Chromium release assay was used. In brief, a standard 4-hour chromium (Cr₅₁) release assay was used to measure the specific cytolytic activity of multi-R-VSTs with autologous antigen-loaded PHA blasts as targets (20 ng/pepmix/1×10⁶ target cells). Effector: Target (E:T) ratios of 40:1, 20:1, 10:1, and 5:1 were used to analyze specific lysis. The percentage of specific lysis was calculated [(experimental release—spontaneous release)/(maximum release—spontaneous release)]×100. In order to measure the autoreactive and alloreactive potential of multi-R-VST lines, autologous and allogeneic PHA blasts alone were used as targets.

Generation of Polyclonal Multi-R-VSTs from Healthy Donors

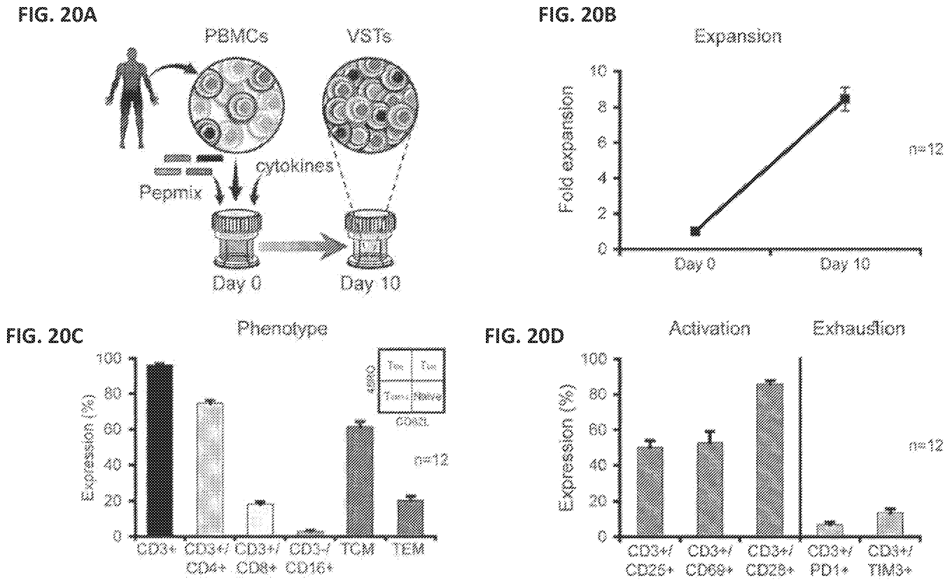

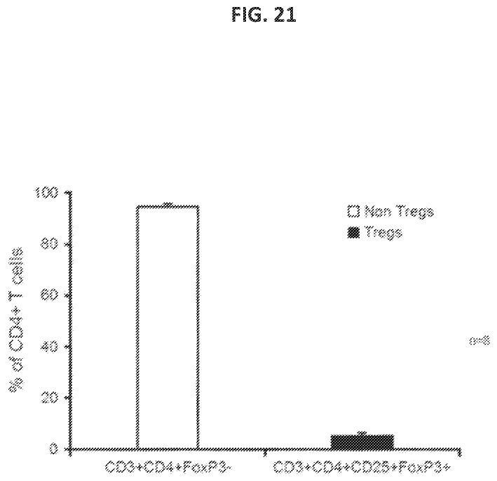

To investigate the feasibility of generating VST-specific T cell lines containing sub-populations of cells reactive against Influenza, RSV, hMPV, and PIV we utilized a pool of overlapping peptide libraries spanning immunogenic antigens from each of the target viruses [Influenza—NP1 and MP1; RSV—N and F; hMPV—F, N, M2-1 and M; PIV—M, HN, N and F] to stimulate PBMCs before culture in a G-Rex10 in cytokine-supplemented VST medium [FIG. 20A]. Over 10-13 days we achieved an average 8.5 fold increase in cells [FIG. 20B] [increase from 0.25×10⁷ PBMCs/cm2 to mean 1.9±0.2×10⁷ cells/cm2 (median: 2.05×10⁷, range: 0.6-2.82×10⁷ cells/cm2 n=12), which were comprised almost exclusively of CD3+ T cells (96.210.6%; mean±SEM), with a mixture of cytotoxic (CD8+; 18.1±1.3%) and helper (CD4+; 74.4±1.7%) T cells [FIG. 20C], with no evidence of regulatory T cell outgrowth, as assessed by CD4/CD25/FoxP3+ staining (FIG. 21).

Furthermore, the expanded cells displayed a phenotype consistent with effector function and long term memory as evidenced by upregulation of the activation markers CD25 (50.2±3.8%), CD69 (52.8±6.3%), CD28 (85.8±2%) as well as expression of central (CD45RO+/CD62L+: 61.4±3%) and effector memory markers (CD45RO+/CD62L−: 20.3±2.3%), with minimal PD1 (6.9±1.4%) or Tim3 (13.5±2.3%) surface expression (FIG. 20 C-D].

Anti-Viral Specificity of Multi-R-VSTs

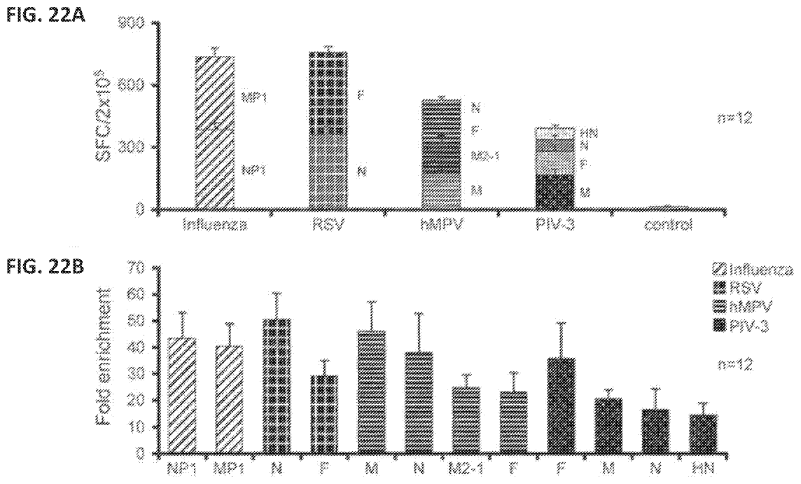

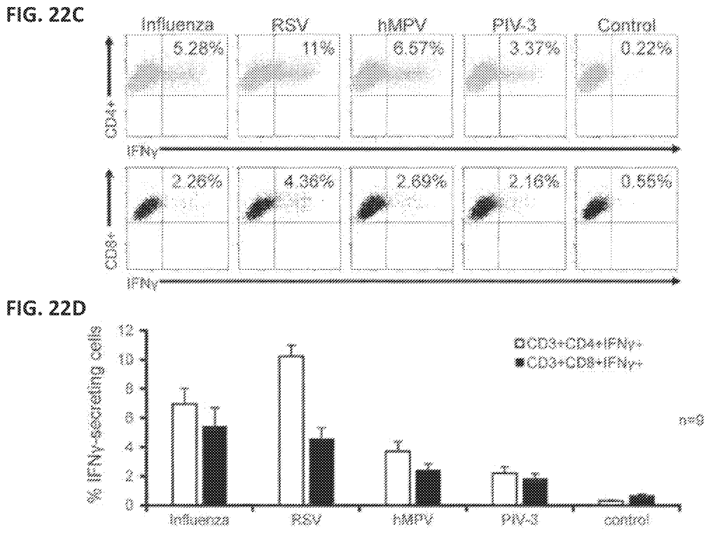

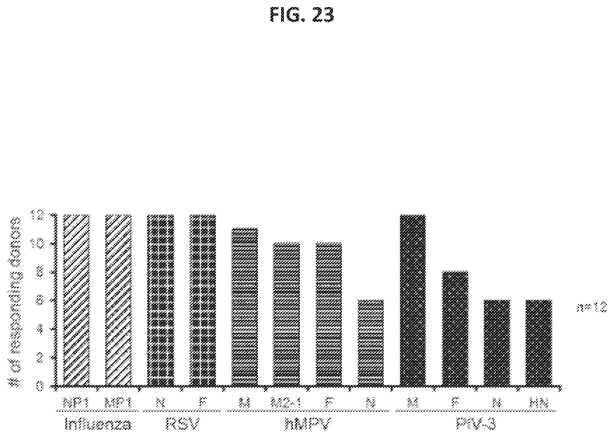

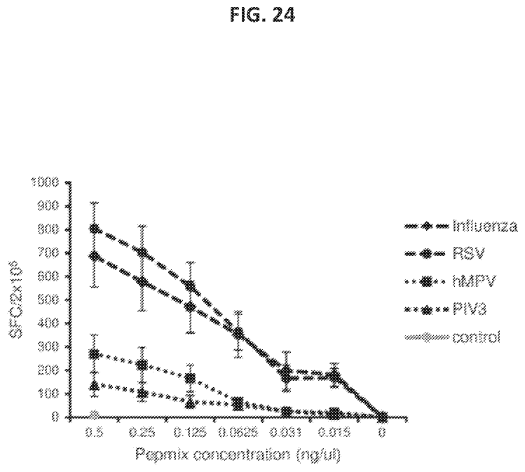

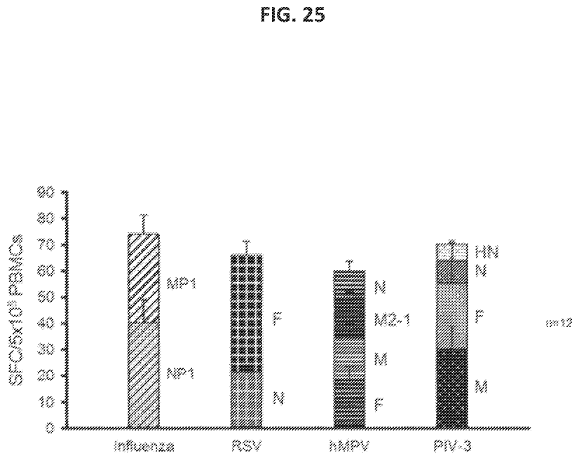

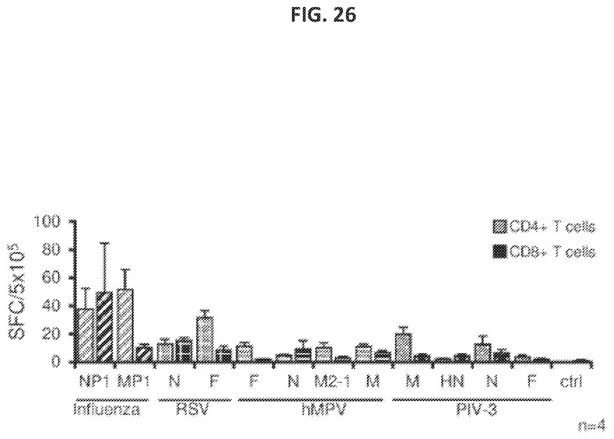

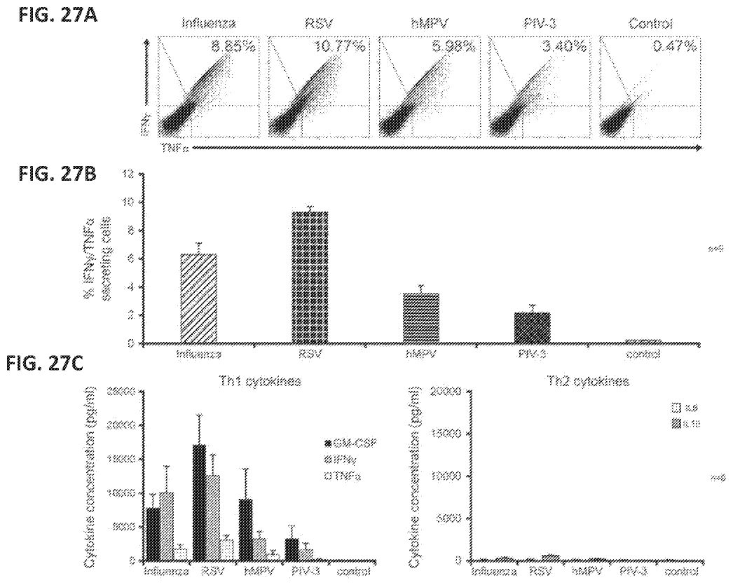

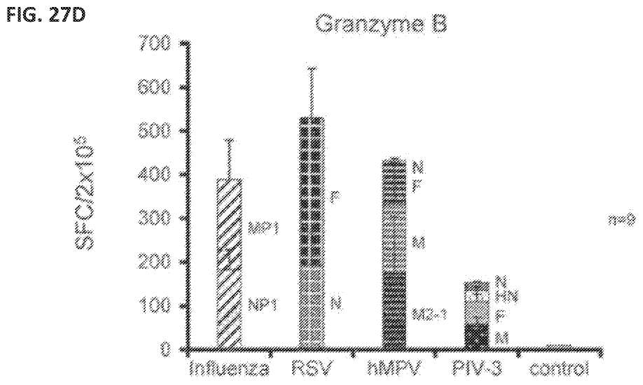

To next determine whether the expanded populations were antigen-specific we performed an IFNy Ellspot assay, using each of the individual stimulating antigens as an immunogen. All 12 lines generated proved to be reactive against all of the target viruses [Table 1, FIG. 23]. FIG. 22A summarizes the magnitude of activity against each of the stimulating antigens, while FIG. 24 shows the response of our expanded VSTs to titrated concentrations of viral antigen. Of note, over the 10-13 days in culture we achieved an enrichment in virus-specific T cells of between 14.6±4.3 (PIV-HN) and 50.4±9.9 fold (RSV-N) [FIG. 22B; the precursor frequencies of CARV-reactive T cells within donor PBMCs are summarized in FIGS. 26 and 27]. Taken together these data suggest that respiratory virus specific T cells reside in the memory pool and can be readily amplified ex vivo using GMP compliant manufacturing methodologies.

To next evaluate whether viral specificity was contained with the CD4+ or CD8+ or both T cell subsets we performed ICS, gating on CD4+ and CD8+ IFNy-producing cells. FIG. 22C shows representative results from 1 donor with activity against all 4 viruses detected in both T cell compartments [(CD4+: Influenza—5.28%; RSV—11%; hMPV—6.57%; PIV—3.37%), (CD8+: Influenza—2.26%; RSV—4.36%; hMPV—2.69%; PIV—2.16%)] while FIG. 22D shows summary results for 9 donors screened, confirming that our multi-R-VST are polyclonal and poly-specific.

Functional Characterization of Multi-R-VSTs

The production of multiple proinflammatory cytokines and expression of effector molecules has been shown to correlate with enhanced cytolytic function and improved in vivo T cell activity. Hence, we next examined the cytokine profile of our multi-R-VSTs following antigen exposure. As shown in FIGS. 27A-27D, the majority of IFNy-producing cells also produced TNFa [FIG. 27A—detailed ICS results from 1 donor; summary results for 9 donors; FIG. 27B], in addition to GM-CSF, as measured by Luminex array [FIG. 27C—left panel] with baseline levels of prototypic Th2/suppressive cytokines [FIG. 27C—right panel]. Furthermore, upon antigenic stimulation our cells produced the effector molecule Granzyme B, suggesting the cytolytic potential of these expanded cells [FIG. 27D, n=9]. Taken together, this data demonstrates the Th1-polarized and polyfunctional characteristics of our multi-R-VSTs.

Multi-R-VSTs are Cytolytic and Kill Virus-Loaded Targets

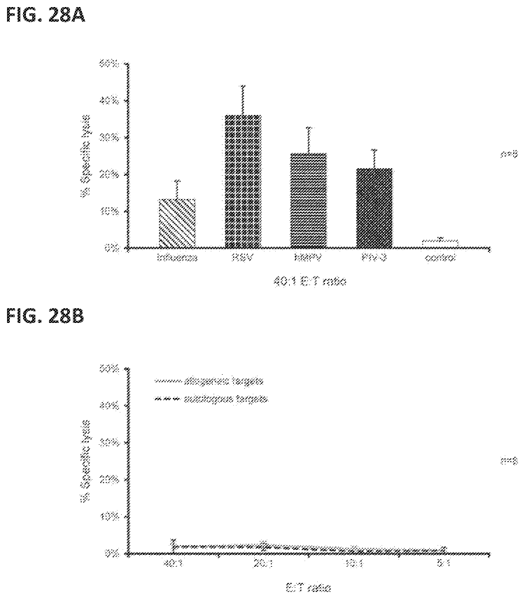

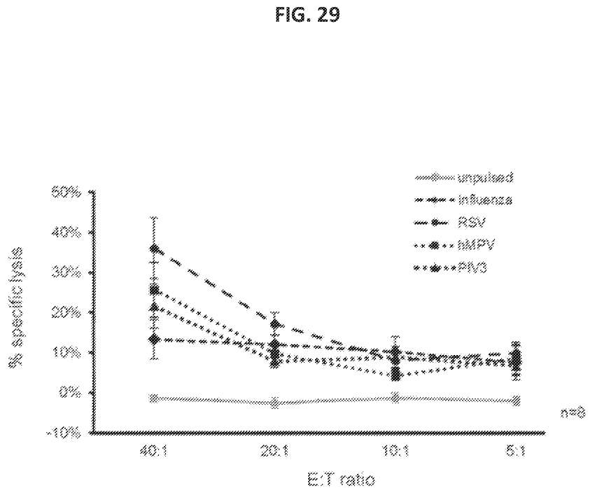

To investigate the cytolytic potential of these expanded cells in vitro we co-cultured multi-R-VSTs with autologous Cr51-labeled PHA blasts, which were loaded with viral pepmixes with unloaded PHA blasts serving as a control. As shown in FIG. 28A and FIG. 29, viral antigen-loaded targets were specifically recognized and lysed by our expanded multi-R-VSTs (40:1 E:T—Influenza: 13±5%, RSV: 36±8%, hMPV: 26±7%, PIV: 22±5%, n=8). Finally, even though these VSTs had received only a single stimulation there was no evidence of activity against non-infected autologous targets nor of alloreactivity (graft versus host potential) using HLA-mismatched PHA blasts as targets (FIG. 28B), which is an important consideration if these cells are to be administered to allogeneic HSCT recipients.

Detection of CARV-Specific T Cells in HSCT Recipients

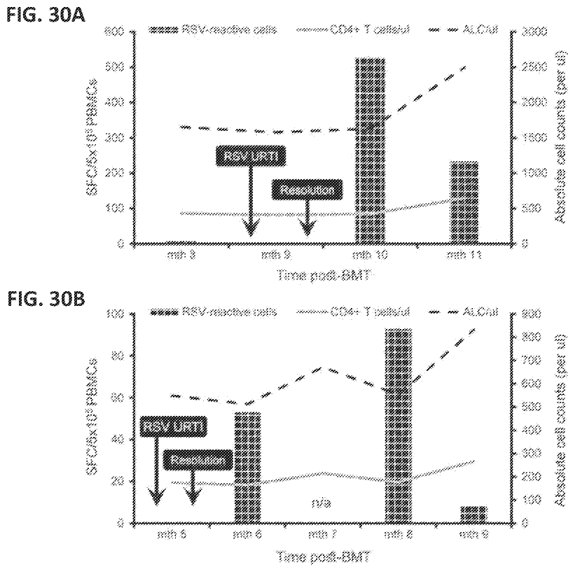

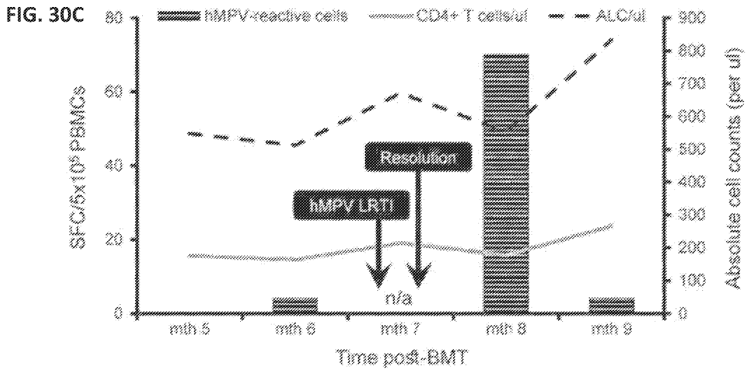

Finally, to assess the potential clinical relevance of multi-R-VSTs we investigated whether allogeneic HSCT recipients with active/recent CARV infections exhibited elevated levels of reactive T cells during/following an active viral episode. FIG. 30A shows the results of Patient #1, a 64-year old male with acute myeloid leukemia (AML) who received a matched related donor (MRD) transplant with reduced intensity conditioning. The patient developed a severe URTI 9 months post-HSCT that was confirmed to be RSV-related by PCR analysis. He was not on any immunosuppression at the time of infection but was placed on prednisone the day of infection diagnosis to control pulmonary inflammation.

Within 4 weeks his symptoms resolved without specific antiviral treatment. To assess whether T cell immunity contributed to viral clearance, we analyzed the circulating frequency of RSV-specific T cells over the course of his infection. Immediately prior to infection this patient exhibited a very weak response to the RSV antigens N and F (6.5 SFC/5×10⁵ PBMCs). However, within a month of viral exposure, RSV-specific T cells had expanded in vivo (527 SFC/5×10⁵ PBMCs), representing an 81-fold increase in reactive cells, as seen in FIG. 30A, which declined thereafter, coincident with viral clearance. Of note, the observed RSV-specific responses did not follow the overall increase in lymphocyte/CD4+ counts, thus indicating that T cell expansion was virus-driven and not due to general immune reconstitution. Similarly, Patient #2, a 23-year old male with acute lymphoblastic leukemia (ALL) who received a matched unrelated donor (MUD) transplant with myeloablative conditioning, and developed a severe RSV-related URTI 5 months post HSCT while on tapering doses of tacrolimus. His infection symptomatically resolved within 1 week, coincident with the administration of ribavirin. To investigate whether endogenous immunity also played a role in viral clearance we monitored reactive T cell numbers over time.

As seen in FIG. 30B, viral clearance was accompanied by an increase in the circulating frequency of RSV-specific T cells (peak 93 SFC/5×10⁵ PBMCs) with subsequent return to baseline levels. The same patient was hospitalized 7 months post-transplant for a subsequent pneumococcal pneumonia with concurrent detection (by PCR) of hMPV in sputum. His pneumonia was treated with antibiotics with subsequent resolution of disease and viral clearance, coincident with a marked expansion of hMPV-specific T cells (reactive against F, N, M2-1 and M), which increased from 4 SFC to a peak of 70 SFC and

subsequent decline to baseline levels (FIG. 30C). Again, the observed RSV- and hMPV-specific responses were independent of the overall increase in lymphocyte/CD4+ counts.

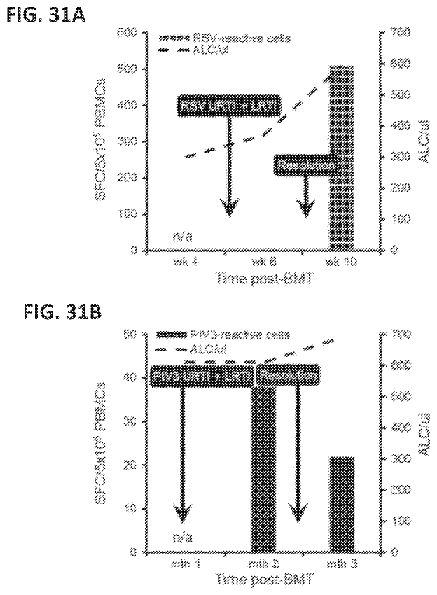

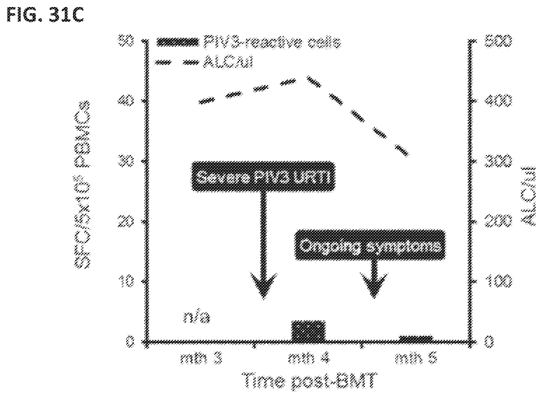

FIG. 31 shows the results of 3 additional HSCT recipients who developed CARV infections. Patient #3, is a 15-year old female with AML who received a haplo-identical transplant with reduced intensity conditioning, and developed an RSV-induced URTI and LRTI while on tacrolimus 5 weeks post-transplant. The patient was administered ribavirin and the infection resolved within 4 weeks. We monitored RSV-reactive T cells over time and, as can be seen in FIG. 31A, viral clearance coincided with a striking increase in the frequency of RSV-specific T cells (from Oto 506 SFC/5×10⁵ PBMCs). Similarly, Patient #4, a 10-year old male patient with ALL who received a MUD transplant with myeloablative conditioning, developed a PIV-related URTI and LRTI 1 month after HSCT while on tacrolimus. His infection symptomatically resolved within 5 weeks, coincident with the administration of ribavirin.

To investigate whether endogenous immunity also played a role in viral clearance, we monitored PIV-reactive T cell numbers over time. As seen in FIG. 31B, viral clearance was accompanied by an increase in the circulating frequency of T cells specific for the PIV antigens M, HN, N and F (peak 38 SFC/5×10⁵ PBMCs) with subsequent decline. Finally, we show Patient #5, a 3-year old male with chronic granulomatous disease who received a MRD transplant with myeloablative conditioning and developed a severe PIV-related URTI 4 months post-HSCT while on cyclosporine. The patient received ribavirin but (at last timepoint assessed) continued to exhibit disease symptoms and failed to demonstrate PIV-specific T cells (FIG. 31C). Taken together, these data suggest the in vivo relevance of CARV-specific T cells in the control of viral infections in immunocompromised patients.

We explored the feasibility of targeting multiple clinically problematic respiratory viruses using ex vivo expanded T cells. We showed that we can rapidly generate polyclonal, CD4+ and CD8+ T cells with specificities directed to a total of 12 antigens derived from 4 seasonal CARVs [Influenza, RSV, hMPV and PIV] that were responsible for upper and lower respiratory tract infections in the immunocompromised host. These broad spectrum VSTs, generated using GMP-compliant methodologies, were Th1-polarized, produced multiple effector cytokines upon stimulation, and killed virus-infected targets without auto-reactivity or allo-reactivity. Finally, the detection of reactive T cell populations in the peripheral blood of allogeneic HSCT recipients who successfully cleared active CARV infections suggests the potential for clinical benefit following the adoptive transfer of such multi-R-VSTs.

CARV-associated acute upper and lower RTls are a major public health problem with young children, the elderly and those with suppressed or compromised immune systems being most vulnerable. These infections are associated with symptoms including cough, dyspnea, and wheezing and dual/multiple co-existing infections are common, with frequencies that may exceed 40% among children less than 5 years and are associated with increased risk of morbidity and hospitalization. Among immunocompromised allogeneic HSCT recipients up to 40% experience CARV infections that can range from mild (associated symptoms including rhinorrhea, cough and fever) to severe (bronchiolitis and pneumonia) with associated mortality rates as high as 50% in those with LRTls. The therapeutic options are limited. For hMPV and PIV there are currently no approved preventative vaccines nor therapeutic antiviral drugs, while the off-label use of the nucleoside analog RBV and the investigation al use of DAS-181 (a recombinant sialidase fusion protein) have had limited clinical impact. The preventative annual Influenza vaccine is not recommended for allogeneic HSCT recipients until at least 6 months post-transplant (and excluded in recipients of intensive chemotherapy or anti-B-cell antibodies), while neuraminidase inhibitors are not always effective for the treatment of active infections.

For RSV, aerosolized RBV is FDA-approved for the treatment of severe bronchiolitis in infants and children, and it is also used off-label for the prevention of upper or lower RTls and treatment of RSV pneumonia in HSCT recipients. However, its widespread use is limited by the cumbersome nebulization device and ventilation system required for drug delivery as well as the considerable associated cost. For example, in 2015 aerosolized RBV cost $29,953 per day, with 5 days representing a typical treatment course. Thus, the lack of approved treatments combined with the high cost of antiviral agents led us to explore the potential for using adoptively transferred T cells to prevent and/or treat CARV infections in this patient population.

The pivotal role of functional T cell immunity in mediating viral control of CARVs has only recently garnered attention. For example, a retrospective study of 181 HSCT patients with RSV URTls, reported lymphopenia (defined as ALC ≤100/mm³) as a key determinant in identifying patients whose infections would progress to LRTI, while RSV neutralizing antibody levels were not significantly associated with disease progression. Furthermore, in a recent retrospective analysis of 154 adult patients with hematologic malignancies with or without HSCT treated for RSV LRTI, lymphopenia was significantly associated with higher mortality rates. Both of these studies are suggestive of the importance of cellular immunity in mediating protective immunity in vivo.

Our group has previously demonstrated the feasibility and clinical utility of ex vivo expanded VSTs to treat a range of clinically problematic viruses including the latent viruses CMV, EBV, BKV, HHV-6 and AdV. Our initial studies (and those of others) explored the safety and activity of donor-derived T cell lines but more recently we have developed an “off the shelf” universal T cell platform whereby VSTs specific for all 5 viruses (CMV, EBV, BKV, HHV-6, AdV) were prospectively generated and banked, thus ensuring their immediate availability for administration to immunocompromised patients with uncontrolled infections.

Indeed, in our recent phase II clinical trial, we administered these partially HLA-matched VSTs to 38 patients with a total of 45 infections that had proven refractory to conventional antiviral agents and achieved an overall response rate of 92%, absent significant toxicity. This precedent of clinical success using adoptively transferred T cells, as well as the absence of effective therapies for a range of CARVs, prompted us to explore the potential for extending the therapeutic scope of VST therapy to Influenza, RSV, hMPV and PIV infections post-HSCT. In this context, one could consider the option of prophylactic VST administration seasonally to high-risk patients [e.g. young (<5 yrs) and elderly adults, patients with impaired immune systems].

Alternatively, these cells could be used therapeutically in patients with URTls who have failed conventional antiviral medications in order to prevent LRT progression. Thus, using our established, GMP-compliant VST manufacturing methodology, we demonstrated the feasibility of generating VSTs reactive against a spectrum of CARV-derived antigens chosen on the basis of both their immunogenicity to T cells and their sequence conservation [Influenza-NP1 and MP1; RSV—N and F; hMPV—F, N, M2-1 and M; PIV—M, HN, N and F from 12 donors with diverse haplotypes. The expanded cells were polyclonal (CD4+ and CD8+), Th1-polarized and polyfunctional, and were able to lyse viral antigen-expressing targets while sparing non-infected autologous or allogeneic targets, attesting to both their virus specificity and their safety for clinical use.

Finally, to assess the clinical significance of these findings we examined the peripheral blood of 5 allogeneic HSCT recipients with active RSV, hMPV and PIV infections. Four of these patients successfully controlled the viruses within 1-5 weeks, coincident with an amplification of endogenous reactive T cells and subsequent return to baseline levels upon viral clearance, while one patient failed to mount an immune response against the infecting virus and has equally failed to clear the infection to date. This data suggests that the adoptive transfer of ex vivo expanded cells should be clinically beneficial in patients whose own cellular immunity is lacking.

In conclusion, we have shown that it is feasible to rapidly generate a single preparation of polyclonal multi-respiratory (multi-R)-VSTs with specificities directed to Influenza, RSV, hMPV and PIV in clinically relevant numbers using GMP-compliant manufacturing methodologies. This data provides the rationale for a future clinical trial of adoptively transferred multi-R-VSTs for the prevention or treatment of CARV infections in immunocompromised patients.

### Example 3. Generation of Donor MiniBanks of Multivirus-Specific T Lymphocytes for the Prevention and Treatment of Infections Following Allo-HSCT

In healthy individuals, T cell immunity defends against BKV and other viruses. In allo-HSCT recipients the use of potent immunosuppressive regimens (and subsequent associated immune compromise) leaves patients susceptible to severe viral infections. Therefore, our approach is to restore T cell immunity by the administration of ex vivo expanded, nongenetically modified, virus-specific T cells (VSTs) to control viral infections and eliminate symptoms for the period until the transplant patient's own immune system is restored. To achieve this goal we have prospectively manufactured VSTs from peripheral blood mononuclear cells (PBMCs) procured from healthy, pre-screened (for infectious agents and disease risk factors as mandated by 21 CFR Part 1271, subpart C), seropositive donors, which are available as a partially HLA-matched “off-the-shelf” product.

One of our VST products (Viralym-M) is specific for five viruses [EBV, CMV, AdV, BKV and Human Herpes virus 6 (HHV6)]. We first set out to construct donor minibanks as described in Example 1 for making Viralym-M cell lines. Our goal was to generate minibanks with sufficient diversity to cover the majority of allogeneic HSCT recipients referred for treatment. Thus, as in the above Examples, we first examined the racial and ethnic diversity of the US transplant population, which we compared with patients who received an allogeneic stem cell transplant at Baylor CCGT (Table 4 and Table 5). This demonstrated that the diversity within the Baylor CCGT patient population is similar if not slightly more diverse than the US population.

TABLE 5
Ethnicity of Allogeneic HSCT Recipients

| | | |
|---|---|---|
| | Total US | Baylor CCGT |
| | (2013-2015) | (2014-2018) |
| Patient Ethnicity | Number (%) | Number (%) |

| | | | |
|---|---|---|---|
| Hispanic or Latino | 2,910 | (12%) | 225 (27.7%) |
| Not Hispanic or Latino | 20,415 | (86%) | 585 (72.3%) |
| Unknown | 521 | (2%) | n/a |
| Total | 23,846 | (100%) | 810 (100%) |

To test our donor selection model described in Example 1, we performed a simulation whereby we compared the HLA types of prospective donors with allogeneic HSCT recipients according to the method in Example 1, and from this identified 25 individuals for inclusion in 5 non-redundant donor minibanks (5 donors per minibank) that would cover >95% of our target patients By constructing these 5 minbanks, we ensured redundancy for each patient (i.e., each patient likely had a suitable match in each of the 5 minibanks).

Table 6 shows the HLA Types of the Viralym-M Donors identified for inclusion in the donor minibanks based on this method.

| Donor ID # | HLA-A | HLA-B | HLA-DR | HLA-DQ |
|---|---|---|---|---|
| 1 | 2.24 | 7.35 | 4.7 | 2.3 |
| 2 | 11.24 | 7.27 | 1.15 | 5.6 |
| 3 | 3.68 | 15.15 | 3.4 | 2.3 |
| 4 | 2.24 | 7.44 | 4.15 | 3.6 |
| 5 | 1.2 | 8.15 | 1.3 | 2.5 |
| 6 | 3.24 | 35.38 | 1.11 | 3.5 |
| 7 | 2.24 | 57.7 | 7.15 | 3.6 |
| 8 | 11.3 | 18.51 | 3.1 | 2.5 |
| 9 | 26.68 | 27.44 | 8.11 | 4.3 |
| 10 | 1.2 | 7.15 | 1.4 | 3.5 |
| 11 | 1.2 | 13.52 | 7.15 | 2.6 |
| 12 | 2.24 | 7.40 | 11.15 | 3.6 |
| 13 | 2.3 | 7.44 | 11.13 | 3.6 |
| 14 | 2.24 | 8.14 | 1.3 | 2.5 |
| 15 | 1.2 | 39.44 | 4.8 | 3.4 |
| 16 | 3.2 | 7.57 | 15.7 | 6.3 |
| 17 | 24.68 | 15.27 | 3.4 | 2.3 |
| 18 | 2.11 | 40.50 | 1.8 | 4.5 |
| 19 | 2.24 | 13.40 | 4.7 | 2.3 |
| 20 | 2.11 | 7.35 | 1.15 | 5.6 |
| 21 | 26.30 | 8.53 | 3.13 | 2.6 |
| 22 | 2.2 | 8.35 | 3.7 | 2.3 |
| 23 | 3.24 | 7.35 | 14.15 | 3.6 |
| 24 | 2.24 | 15.39 | 1.4 | 3.5 |
| 25 | 68.2 | 7.57 | 15.7 | 6.3 |

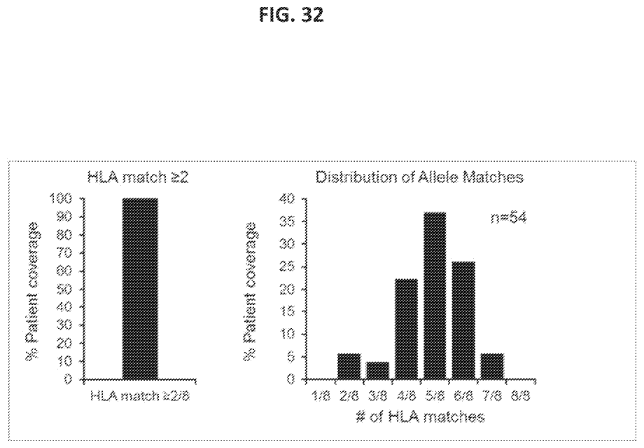

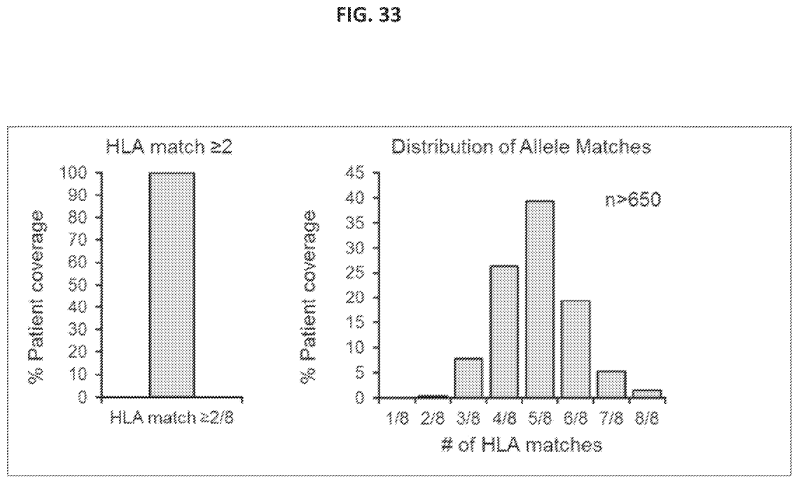

To formally assess whether our simulated Viralym-M donor bank (Table 6) would indeed provide the stated coverage we first evaluated the potential of this bank to accommodate patients enrolled in our POC Phase II study with a potent T cell product matched on at least 2 HLA alleles. As shown in FIG. 32 we were indeed able to accommodate all 54 patients (100%) with a product matched on at least 2 HLA alleles and achieved a mean of 5±1 shared alleles (range 2-7/8 matched alleles). Furthermore, when we extended this analysis to our entire >650 Baylor CCGT allogeneic HSCT patient population we were again able to accommodate 100% of all prospective patients with a product matched on at least 2 HLA alleles and again achieved a mean of 5±1 shared alleles (range 2-8/8 matched alleles)(FIG. 33).

Taken together, these data supports that our donor minibanks (containing virus specific T cell lines generated from carefully selected donors) can provide cover to at least 95% of the US allogeneic HSCT patient population with a product matched at a minimum of 2 HLA alleles.

The Viralym-M manufacturing process was as previously described by the inventors in WO2013/119947 and Tzannou et al., J Clin Oncol. 2017 Nov. 1; 35(31: 3547-3557, each of which is incorporated herein by reference in its entirety and is outlined in FIG. 12. Briefly, PBMCs were isolated from healthy seropositive donors and 250×10⁶ PBMCs were cultured in a G-Rex 100M culture system (Wilson Wolf, Saint Paul, MN) in the presence of complete medium, pepmixes covering the Viralym M antigens (adenovirus, CMV, EBV, BKV, and HHV6), IL-4, and IL-7 for around 7-14 days at 37 degrees C. at 5% CO₂ (although the culture time may be increased to around 18 days in some instance). After culturing, Viralym M cell lines were harvested, washed, and aliquoted for cryopreservation in liquid nitrogen until use in quality control testing or as a therapeutic.

FIG. 13 shows the respective potency of the antigen-specific T cell lines against adenovirus, CMV, EBV, BKV, and HHV6 compared with the negative control, which is below the potency threshold. The T cells are specific for all five viruses as indicated by >30 SFC/2×10⁵ input VSTs, which is the threshold for discriminating between acceptance and rejection of a specific T cell line. The potency threshold of >30 SFC/2×10⁵ input VSTs was established based on experimental data using T cell lines generated from donors that were seronegative (based on serological screening) for one or more of the target viruses, which served as an internal negative control (FIG. 14).

We evaluated Viralym-M in a Phase 2 open-label proof-of-concept trial where VSTs were administered to 58 allogeneic HSCT patients with treatment-refractory infections. We refer to this trial as CHARMS. Data from this study is report in (Tzannou et al, JCO, 2017).

The primary objective of CHARMS, which was not statistically powered for superiority or significance, was to determine the feasibility and safety of administering partially HLA-matched multi-VST therapies specific for five viruses in HSCT patients with persistent viral reactivations or infections. Patients were eligible following any type of allogeneic transplant if they had BKV, CMV, AdV, EBV, HHV-6 and/or JCV infections that were relapsed, reactivated or persistent despite standard antiviral therapy.

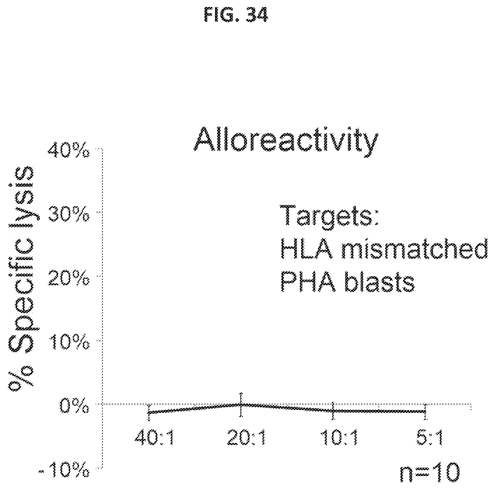

To assess the alloreactive potential of multivirus-specific T cells (Viralym-M cells) we first directly activated PBMCs with peptide mixtures spanning immunogenic antigens derived from each virus;—Adv (Hexon and Penton), CMV (IE1 and pp65), EBV (LMP2, EBNA1, BZLF1), BK virus (VP1 and large T), and HHV6 (U90, U11 and U14). We then transferred cells to the G-Rex device in T cell medium supplemented with IL4+7 (as described in FIG. 12) and assessed their cytotoxic activity against HLA-mismatched targets. As shown in FIG. 34 these cells exhibited minimal/no detectable alloreactivity, supporting the potential safety of these cells when administered as an “off the shelf” partially HLA matched product.

We subsequently explored the safety and clinical effects of partially HLA-matched Viralym-M cells for the treatment of refractory viral infections in children and adults following allogeneic HSCT (Tzannou et al, JCO, 2017).

All infusions were well tolerated. Except for 3 patients who developed a transient fever and one who developed lymph node pain within 24 hours of infusion, no acute toxicities were observed. None of the patients developed cytokine release syndrome (CRS). In the ensuing weeks after infusion, one patient developed recurrent Grade III gastrointestinal (GI) GVHD following rapid steroid taper, and eight patients developed recurrent (n=4) or de novo (n=4) Grade I-II skin GVHD, which resolved with the administration of topical treatments (n=7) and re-initiation of corticosteroids after taper (n=1).

Clinical effects:

For sixty infections in the 52 treated patients who provided evaluable data, the cumulative clinical response rate was 93% by week 6 post Viralym-M infusion, as summarized below:
BKV: Twenty-two patients received Viralym-M for the treatment of persistent viral BKV infection and tissue disease (20 with BK-hemorrhagic cystitis and 2 with BKV-associated nephritis). All 20 BK-HC patients had resolution of clinical symptoms after receiving Viralym-M with 9 complete responses (CRs) and 11 partial responses (PRs), for a 6-week cumulative response of 100%.
CMV: Twenty patients received Viralym-M for persistent CMV. 19 patients responded to Viralym-M with 7 CRs and 12 PRs with 1 non-responder (NR), for a 6-week cumulative response rate of 95%. Responders included 2 of 3 patients with colitis and 1 patient with encephalitis.
AdV: Eleven patients received Viralym-M for persistent AdV and infusions produced 7 CRs, 2 PRs, and 2 NRs, with a 6-week cumulative response rate of 81.8%.
EBV: Three patients received Viralym-M for the treatment of persistent EBV. Two patients achieved a virologic CR and one patient a PR.
HHV6: Four patients received Viralym-M to treat HHV6 reactivations including one patient with refractory encephalitis, and three patients had a PR within 6 weeks of infusion (including the patient with encephalitis) while one did not respond to the treatment.
Dual infections: Eight patients received Viralym-M for two viral infections, with an overall experience of 12 CRs and 4 PRs following a single infusion. CMV, AdV, and EBV were cleared in all cases, all patients with BKV HC had clinical improvement (n=3) or disease resolution (n=2) and the patient with HHV6 encephalitis also had clinical improvement.

We examined the data available from our Phase 1/II Viralym-M study to determine whether there was a threshold of HLA matching associated with clinical efficacy. On our clinical trial the products that were used clinically were matched at 1/8 (n=2), 2/8 (n=10), 3/8 (n=11), 4/8 (n=14), 5/8 (n=14), 6/8 (n=4), or 7/8 (n=5) HLA alleles. To determine whether there was a correlation with clinical outcome and degree of HLA matching, we segregated patients into complete response (CR), partial response (PR), and non-responders (NR), but as summarized in FIG. 35, the results suggest that there was no difference in outcome based on the number of HLA matching alleles.

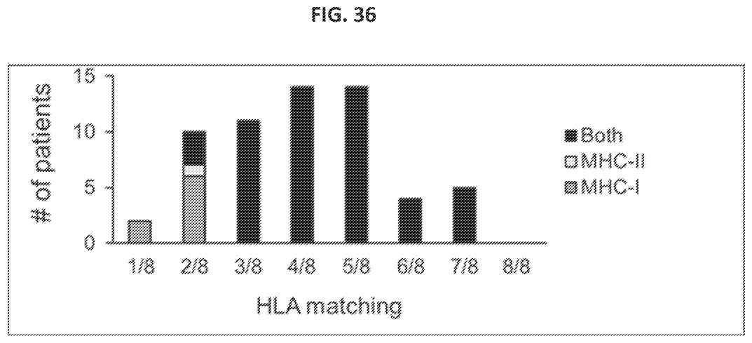

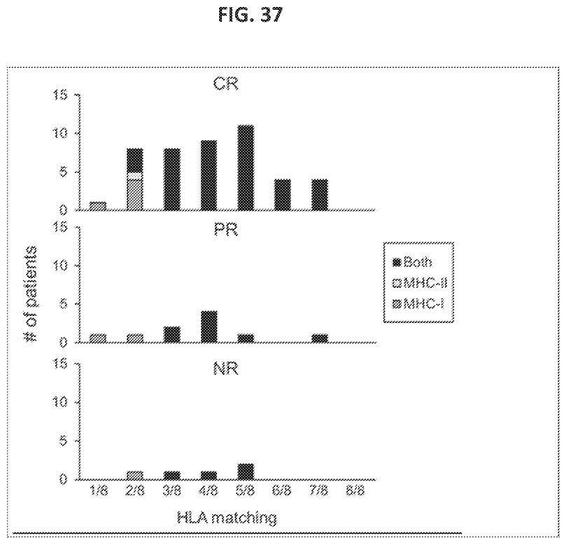

We next examined whether there was a difference in outcome based on the administration of lines matched at HLA class I only, class II only, or a combination of both. Of note, the majority of patients received lines that were matched on both class I and class II alleles (FIG. 36) and again the results suggest that outcome was not influenced by degree of allele matching (FIG. 37).

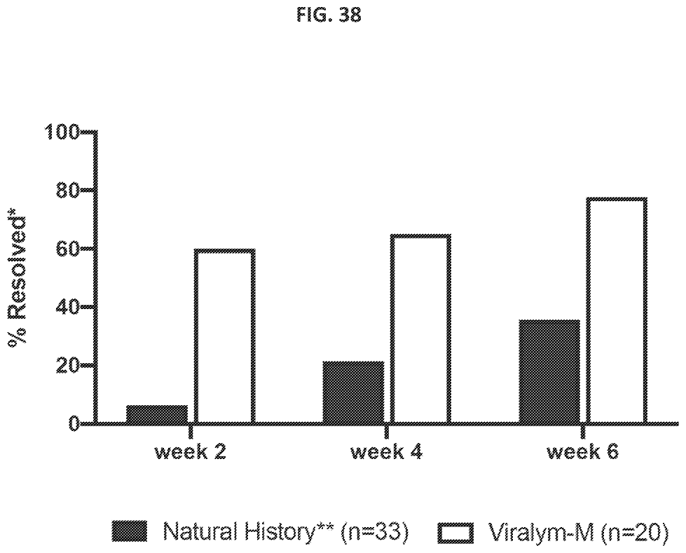

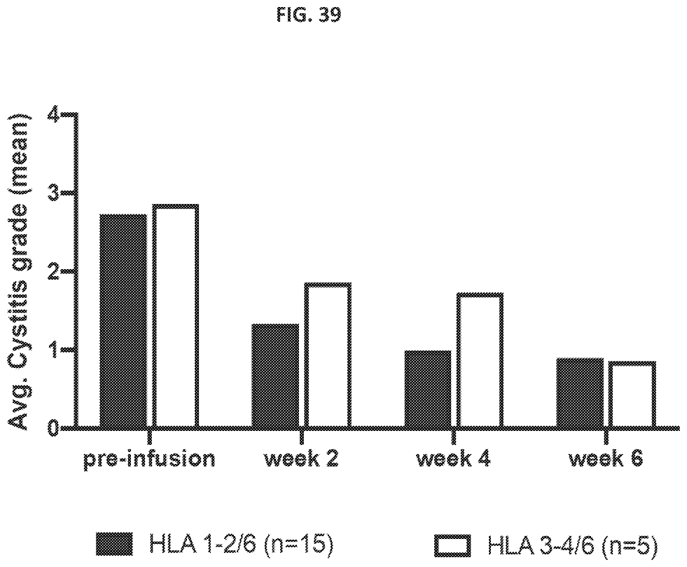

As noted above, the primary clinical manifestation of BKV infection following allogeneic HSCT is hemorrhagic cystitis (BK-HC). In the CHARMS study, 26 patients with virus induced hemorrhagic cystitis were treated with the VSTs as provided above, and patients were graded for hemorrhagic cystitis over time at 0, 2, 4, and 6 weeks after infusion. The mean pre-infusion cystitis grade was 2.81±0.08. Across all 24 patients in the study who were eligible for cystitis grading, BK-HC was rapidly resolved. Resolution was defined as ≤grade 1. As shown in FIG. 37, resolution was achieved in 50% of VST recipient patients by 2 weeks post-infusion and in 75% of patients by 6 weeks post-infusion. FIG. 38 shows the rapid resolution of cystitis in the 20 patients over time, compared to historic data from 33 HSCT patients with an average of Grade 3 BK-HC that received standard of care treatment (no VSTs). Notably, when the VST recipients were divided into low-level HLA-match groups (1-2 of 6 matches) and higher level HLA-match groups (3-4 of 6 matches), the reduction in average cystitis grade was similar in both groups (FIG. 39). Thus, VSTs are effective even in low level HLA-match settings.

Moreover, importantly, the CHARMS study demonstrated that it is safe and efficacious to administer more than one different VST product (Viralym M), even if the second line is highly mismatched. For example, as is reported in Table 2 of Tzannou (2017), several patients received administration of two separate cell lines with beneficial responses:

TABLE 7
Selected patient responses (modified from Table 2 in Tzannou (2017).

| Patient | | Lines | HLA Matching | Best Response | |
|---|---|---|---|---|---|
| No. | Infection | Infused | (of eight lines) | by 6 Weeks | Outcome |
| 3848 | resistant | C5404; | 3 alleles; | PR; no | PR with recurrence at 4 |
| | strain | C5678 | 4 alleles | | weeks |
| | CMV | | | | |
| 3357 | CMV | C5678; | 4 alleles; | PR | Sustained CR |
| | | C6323 | 5 alleles | | |
| 4076 | CMV, | C6209; | 6 alleles; | CMV CR; | Sustained CR for CMV; |
| | AdV | C6611 | 3 alleles | AdV CR | recurrence of AdV with |
| | | | | | sustained CR after |
| | | | | | second infusion |
| 3755 | EBV, | C5602, | 5 alleles; | EBV CR; | Sustained CR for EBV; |
| | BKV | C5624 | 2 alleles | BKV PR | PR for BKV with stable |
| | | | | | renal function |
| 3877 | BKV | C6322, | 3/6 alleles; | Virologic PR; | Resolution of HC after |
| | | C5602 | 4 alleles | Clinical PR | third infusion |
| 3899 | BKV | C6726, | 4 alleles; | Virologic PR; | Resolution of HC after |
| | | C5497 | 3 alleles | Clinical PR | second infusion |

Moreover, as shown in Table A7 of Tzannou (2017), modified below in Table 8, these patients that received administration of at least two cell lines showed no or little aGVHD by week 6 or cGVHD within 1 year of treatment.

TABLE 8
Selected patient responses (modified
from Table 2 in Tzannou (2017).

| Patient | | aGVHD by Week 6 | cGVHD Within 1 Year |
|---|---|---|---|
| No. | Infection | (treatment; outcome) | (treatment; outcome) |
| 3848 | resistant | NO | N/ANO |
| | strain CMV | | |
| 3357 | CMV | Grade 1 skin (topical | NO |
| | | corticosteroids; resolved) | |
| 4076 | CMV, AdV | NO | NO |
| 3755 | EBV, BKV | NO | quiescent chronic |
| | | | GVHD |
| 3877 | BKV | Grade 1 skin (topical | NO |
| | | corticosteroids; resolved) | |
| 3899 | BKV | NO | N/A |

Abbreviations: GVHD: graft versus host disease; aGVHD: acute GVHD; cGVHD: chronic GVHD; N/A: not applicable.

Thus, these results from this Phase I/II data demonstrate that >95% of patients received a product matching at ≥2 HLA alleles, which was associated with clinical benefit. Matching on HLA class I or class II did not appear to influence outcome and did not impact the safety profile of the cells, nor did administering more than one cell line to a given patient, even when second line was highly mismatched.

### Example 4. Universal Cell Therapy Products

As is discussed above, when administered to allogeneic HSCT recipients using HLA match (≥2 allele threshold) as the criterion for line selection these cells proved safe and provided antiviral activity against all 5 of the target viruses in our POC clinical trial (Example 3, and clinical trial identifier NCT02108522). Furthermore, infusion of multiple different cell line products was well tolerated, even when the cell line had a high degree of mismatch (see e.g., patient number 3755, who was infused a second cell line that was matched at only 2 alleles (vs a first line that was matched at 5 alleles).

These results suggest that there is little to no risk at administering to a single patient multiple cell lines with variant degrees of allelic match. Based on these results, a universal cell therapy product is prepared by pooling all of the cell lines in a given donor minibank. because each minibank covers >95% of the target patient population, such a universal cell therapy product contains a matching cell therapy product for >95% of prospective patients. Thus, in some instances the universal cell therapy product is administered to a subject in need thereof irrespective of the subject's HLA type. In some instances, the universal cell therapy product is administered to a subject in need thereof who has an HLA match on at least 2 alleles with at least one cell line in the universal cell therapy product. The subject may be an HSCT recipient.

In some instances, a plurality of cell therapy products in a donor minibank are administered to a subject sequentially. For example, in one instance, all of the cell therapy products in a donor minibank are administered to a single subject in need thereof.

### Example 5. Method of Matching a Patient to the Best Suited Cell Line in a Donor Minibank

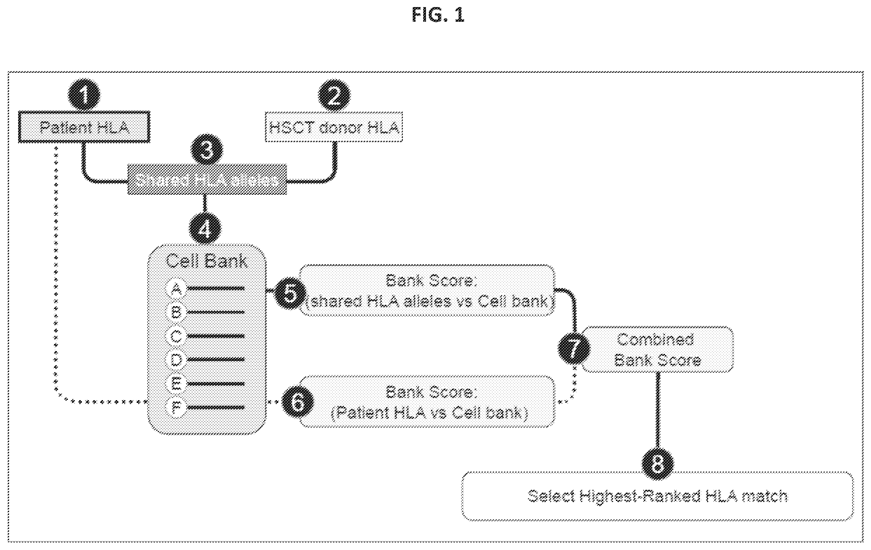

To ensure that best possible cell therapy product in a donor minibank is administered to a given patient, we developed a patient match algorithm, which choses the highest overall level of HLA match between the banked Viralym-M cells and (a) the HSCT patient, and (b) their stem cell donor as summarized in FIG. 1. Specifically, the algorithm implements the following step-wise process:

### Steps:

1. Obtain documentation containing the patient's HLA type;
2. Obtain documentation containing the stem cell donor's HLA type (hereafter referred to as “Transplant HLA”);
3. Compare patient's HLA (step 1) and Transplant HLA (step 2) types and identify shared HLA alleles;
4. Access the HLA types of the individual lines that constitute the donor minibank (e.g., Viralym-M);
5. Primary score: Compare the HLA types of each cell line in minibank (e.g., Viralym-M) with the shared HLA alleles identified in Step 3. Each comparison is assigned a numerical score based on the number of shared HLA alleles; wherein the more alleles shared the higher the score;
6. Secondary score: Compare the HLA types of each cell line in minibank (e.g., Viralym-M) with the patient HLA (representing the infected tissue) identified in Step 1. Each comparison is assigned a score based on the number of shared HLA alleles—the more alleles shared the higher the score. This secondary score is weighted at 50% of the primary score;
7. The primary (Step 5) and secondary score (Step 6) for each line within the cell bank are added together;
8. The cell line (e.g., Viralym-M) with the highest score based on ranking above (Step 7) is then selected for the treatment of the patient.

When clinically applied this approach has demonstrated an acceptable safety profile (4 cases of de novo grade I-II skin GVHD and one grade 3 GI GVHD flare) and proof of concept for the treatment of infection and disease with a 93% response rate achieved in 54 patients treated to date. Table 9 summarizes the safety and clinical outcomes of all 54 patients treated with 3^{rd} party Viralym-M cells in the inventor's Phase 1/II clinical trial.

TABLE 9
Safety and Clinical Effects of Viralym-M in Children and Adults

| | | | Infusion | | | |
|---|---|---|---|---|---|---|
| | | | Related | Acute | | |
| | | Non- | Toxicity | GVHD | Chronic | |
| | | Hematological | (within | (Grade | GVHD | Clinical |
| Pt. ID | Age | AE (Grade 3-5) | 24 hours) | III-IV) | Month 3-12 | Response |
| 4002 | 2 | | | | | CR |
| 4243 | 3 | Grade 4 | | | | PR |
| | | respiratory | | | | |
| | | failure/hypoxia | | | | |
| | | (possibly | | | | |
| | | related) | | | | |
| 4268 | 3 | | | | | CR |
| 3357 | 4 | | | | | CR |
| 3899 | 5 | | Fever | | | CR |
| | | | (possibly | | | |
| | | | related) | | | |
| 3809 | 6 | | Fever | | | PR |
| | | | (probably | | | |
| | | | related) | | | |
| 4108 | 9 | | | | | CR |
| 4057 | 10 | | | | | lost to follow-up |
| 3854 | 10 | | | | | NE |
| 3877 | 12 | | | | mild limited | CR |
| | | | | | chronic skin | |
| | | | | | GVHD, not | |
| | | | | | requiring | |
| | | | | | treatment | |
| 4084 | 14 | | | | | PR |
| 4134 | 15 | | | Grade III | | CR |
| | | | | (GI flare) | | |
| 4155 | 15 | | | | | CR |
| 4183 | 16 | | | | | CR |
| 3864 | 16 | | | | | CR |
| 4183 | 17 | Grade 3 blurry | | | | PR |
| | | vision | | | | |
| | | (neurological- | | | | |
| | | possibly related) | | | | |
| | | (neurological- | | | | |
| | | possibly related) | | | | |
| 3902 | 18 | | | | Flare of UGI | CR |
| | | | | | GVHD 5 mo | |
| | | | | | post-infusion | |
| | | | | | after stopping | |
| | | | | | budesonide, | |
| | | | | | responded to | |
| | | | | | restarting | |
| | | | | | steroids 1 | |
| | | | | | mg/kg | |
| 4266 | 18 | | Grade I | | | CR |
| | | | lymph node | | | |
| | | | pain | | | |
| | | | (possibly | | | |
| | | | related) | | | |
| 4271 | 18 | | | | | PR |
| 3827 | 19 | | | | | CR |
| 4168 | 20 | | | | | CR |
| 4281 | 22 | | | | | PR |
| 3755 | 23 | | | | | PR |
| 4206 | 23 | | | | | NR |
| 4224 | 25 | | | | | CR |
| 3840 | 26 | | | | | CR |
| 3810 | 25 | | | | | PR |
| 3904 | 26 | | | | | CR |
| 4198 | 29 | | | | | CR |
| 3859 | 31 | | | | | PR with |
| | | | | | | improved renal |
| | | | | | | function |
| 3750 | 36 | | | | | CR |
| 3868 | 37 | | | | | PR |
| 3908 | 39 | | | | | CR |
| 3929 | 43 | | | | | CR |
| 4021 | 44 | | Fever | | | NR |
| | | | (possibly | | | |
| | | | related) | | | |
| 4126 | 45 | | | | | CR |
| 4234 | 50 | | | | | CR |
| 3967 | 51 | | | | | CR |
| 3848 | 54 | | | | | NR |
| 4204 | 55 | | | | | CR |
| 4056 | 55 | | | | | CR |
| 4157 | 56 | | | | | CR |
| 3796 | 58 | | | | | CR |
| 3843 | 59 | | | | | CR |
| 4245 | 59 | | | | | CR |
| 3870 | 59 | | | | Flare of UGI | CR |
| | | | | | GVHD after | |
| | | | | | taper of | |
| | | | | | budesonide, | |
| | | | | | responded to | |
| | | | | | short | |
| | | | | | prednisone | |
| | | | | | course | |
| 2936 | 60 | | | | | CR |
| 4076 | 62 | | | | | CR |
| 3869 | 63 | | | | | NR |
| 3924 | 64 | | | | | CR |
| 3784 | 65 | | | | | CR |
| 3921 | 65 | | | | | CR |
| 3830 | 68 | | | | | CR |
| 4193 | 73 | | | | | CR |

Thus, these data demonstrate that the Viralym M products from our minibank were well tolerated and effective when administered to a well-matched patient using our patient match algorithm.

### Example 6. Generation and Testing of a Universal Antigen-Specific T Cell Therapy Product

FIG. 40 provides a schematic illustration of the generation and use of a universal antigen-specific T cell therapy product, compared to prior methods for preparing and using individual antigen-specific T cell therapy products.

A study was conducted to prepare a universal antigen-specific T cell product by pooling individual VST products from donors with distinct HLA types. In the study, experiments were conducted to establish potency and safety of the pooled product (referred to herein as UVSTs). For example, potency was measured by IFNγ ELISPOT. In addition, the IFNγ ELISPOT was used to confirm that the pooled product contained cells from each of the individual VST lines, which recognized HLA restricted epitope peptides unique to the individual lines. Finally, safety was measured by a lack of alloreactivity. Autoreactivity was also measured.

The individual donor products were prepared for 3 donors with disparate HLA types, as shown in below in Table 10. The individual cell lines were prepared as previously described, e.g., in WO2013/119947 and Tzannou et al., J Clin Oncol. 2017 Nov. 1; 35(31: 3547-3557), each of which is incorporated herein by reference in its entirety and is outlined in FIG. 12. Briefly, PBMCs were isolated from healthy seropositive donors and 250×10⁶ PBMCs were cultured in a G-Rex 100M culture system (Wilson Wolf, Saint Paul, MN) in the presence of complete medium, pepmixes covering adenovirus, CMV, EBV, BKV, and HHV6 antigens, IL-4, and IL-7 for 14 days at 37 degrees C. at 5% CO₂.

TABLE 10
Donor HLA types

| | HLA | | |
|---|---|---|---|
| | A | B | DR |
| Donor 1 | 01.24 | 08.18 | 1 |
| Donor 2 | 02.24 | 13.35 | 12.15 |
| Donor 3 | 02.02 | 07.15 | 09.11 |

Unique immunogenic HLA-restricted epitopes (UE) were identified for each donor to facilitate tracking of each line once the cell lines were pooled:
UE1—HHV6: U 11: Epitope peptide 168 (HLA-A1-restricted—unique to Donor 1)
UE2—HHV6: U14: Epitope peptide 102 (HLA-A2-DR15-restricted—unique to Donor 2)
UE3—HHV6: U14: Epitope peptide 40 (HLA-A2-DR11-restricted—unique to Donor 3)

15×10e6 VSTs were pooled from each donor product and combined for freeze as a UVST product with a total of 45×10e6VSTs/vial (1:1:1 ratio). Concurrently, individual cell line products from each donor were frozen at 15×10e6 VSTs/vial.

Vials of pooled UVST product and individual VST cell lines were thawed and rested overnight, then tested for identity and potency by IFNγ ELISpot, and for auto- and allo-reactivity by chromium release assay.

IFNγ ELISpot was performed on thawed (individual and pooled) VSTs to assess the potency against the following viral antigens and unique epitope (UE) peptides:
Viruses (antigens)—ADV, BKV, CMV, EBV, HHV6
Unique (tracking) epitope peptides—UE1, UE2, UE3
Controls:
   Irrelevant antigen—Survivin
   Irrelevant peptide—Peptide 183;
   Negative control—Medium only
   Positive control—PHA

For UE wells, UVSTs were plated at 6×10e5/well. For the 5 virus antigens and the controls, UVSTs were plated at 2×10e5/well. For testing of individual donor product potency, individual donor products were plated at 2×10e5/well for each of the 5 virus antigens, controls, and UEs. Spot Forming Units (SFUs)/2×10e5 VSTs/well were quantitated using Mabtech IRIS reader. The results of the study are provided in Table 11. UVSTs produced IFNγ in response to each viral antigen and each Donor UE. Thus, the identity of each of the individual VST cell lines was confirmed in the UVST product by IFNγ ELISpot assay, indicating that universal VSTs are potent post-thaw.

TABLE 11
Results of IFNγ ELISPOT
Table 1. (SFUs/2 × 10⁵ VSTs)

| | UVSTS |
|---|---|
| ADV | 2609 |
| BKV | 1066 |
| CMV | 475 |
| EBV | 1416 |
| HHV6 | 1999 |

| | UVST | Donor 1 | Donor 2 | Donor 3 |
|---|---|---|---|---|
| Donor 1 UE (168) | 336 | 404 | 0 | 3 |
| Donor 2 UE (102) | 425 | 0 | 411 | 0 |
| Donor 3 UE (40) | 1919 | 0 | 0 | 2340 |
| Irrelevant Antigen (SURV) | 0 | 0 | 0 | 1 |
| Irrelevant Peptide (183) | 0 | 0 | 0 | 0 |
| Negative Control | 0 | 0 | 0 | 0 |
| Positive Control | 1750 | 1486 | 1685 | 2075 |

Chromium release assays were performed to assess auto- and allo-reactivity of the UVSTs, against autologous PHA blasts or allogeneic PHA blasts. To generate PHA blasts, PBMCs were stimulated with hytohemagglutinin (PHA) in the presence of IL-2. UVST cells were used as effectors. Targets were autologous PHA blasts from each donor individually (donor 1, 2, and 3), or PHA blasts from an unrelated donor (donor 4, shown below in Table 12 along with the 3 donors as provided above). Cells were plated at an effector-to-target ratio of 40:1 (6×10e5 UVST effectors to 5×10e3 targets).

TABLE 12
Donor HLAs including unrelated Donor 4

| | HLA | | |
|---|---|---|---|
| | A | B | DR |
| Donor 1 | 01.24 | 08.18 | 1 |
| Donor 2 | 02.24 | 13.35 | 12.15 |
| Donor 3 | 02.02 | 07.15 | 09.11 |
| Donor 4 | 03.03 | 07.27 | 11.15 |

The results are provided in FIG. 41. There was no auto-reactivity against autologous blasts. Further, there was no allo-reactivity against the unrelated donor

Thus, taken together, the results of the study demonstrated that UVSTs are potent across antigen specificities and lack allo-reactivity against donor cells including unrelated donor cells, and thus are suitable for use as a universal cell therapy product.

### Example 7. Safety and Potency of UVSTs after Product Re-Freeze

A study was conducted to determine if a thawed UVST product was safe and effective after the product was pooled together from frozen and thawed cell lines, and refrozen as a combined universal product. Safety was assessed by alloreactivity; efficacy was assessed by viability and specificity of T cells in the product.

The manufacture of the product was conducted as set forth in Example 6, using the same donors provided in Table 12. After generation of the 3 individual VST cell lines (from Donors 1, 2, and 3), the individual cell lines were cryopreserved at 15×10e6 VSTs/vial. The following day, a vial from each of the 3 donor products was thawed and rested overnight. Cells were resuspended in VST medium and supplemented with 10 ng/mL IL-7 and 400 U/mL IL-4; and transferred to a 24 well plate for overnight incubation at 37° C., 5% CO₂. Post-rest, VSTs from each donor (5×10e6 cells each) were pooled together at a 1:1:1 ratio, for a total of 15×10e6 VST/s vial. The pooled product was then cryopreserved as a combined universal product (UVST).

Subsequently, vials of the pooled UVST product, as well as the frozen individual VSTs, were thawed. Potency and identity were assessed by IFNγ ELISPOT. UVSTs were plated with the 5 viral antigens (ADV, BKV, CMB, EBV, and HHV6), unique tracking epitope peptides (UE1, UE2, and UE3), and controls as described above in Example 6. Table 13 provides the results of the study, which showed that the pooled, refrozen and thawed UVSTs produced IFNγ in response to each viral antigen and each Donor UE. Thus, the identity and potency of each of the individual VST lines was maintained within the frozen and thawed UVST product generated from thawed individual cell lines.

TABLE 13
(SFUs/2 × 10⁵ VSTs)

| | UVSTs |
|---|---|
| ADV | 3620 |
| BKV | 1416 |
| CMV | 391 |
| EBV | 1610 |
| HHV6 | 3796 |

| | UVST | Donor 1 | Donor 2 | Donor 3 |
|---|---|---|---|---|
| Donor 1 UE (168) | 442 | 563 | 6 | 0 |
| Donor 2 UE (102) | 472 | 4 | 416 | 3 |
| Donor 3 UE (40) | 2014 | 0 | 0 | 3072 |
| Irrelevant Antigen (SURV) | 0 | 4 | 0 | 0 |
| Irrelevant Peptide (183) | 9 | 0 | 5 | 0 |
| Negative Control | 0 | 0 | 0 | 0 |
| Positive Control | 2110 | 1665 | 1646 | 1559 |

The results of the study show that individual VST lines can be generated and assessed to confirm identity, potency, and/or other quality control parameters, and then frozen prior to pooling; and that subsequently, the individual cell lines can be thawed and pooled together to generate a universal VST product, which can then be frozen for later use. Accordingly, existing banks of individual VST products can be thawed and pooled together to generate a UVST, which can then be cryopreserved for later use.

1. A population of antigen-specific T cells comprising a plurality of antigen-specific T cell lines derived from a plurality of different donors, wherein the HLA type of each donor differs from at least one of the other donors on at least one HLA allele.

2. The population of antigen-specific T cells of claim 1, wherein the antigen-specific T cell lines are clonal, oligoclonal, or polyclonal.

3. The population of antigen-specific T cells of claim 1 or claim 2, wherein the HLA type of each donor differs from at least one of the other donors on at least two HLA alleles.

4. The population of antigen-specific T cells of any one of claims 1-3, wherein the HLA type of each donor differs from at least one of the other donors on at least three HLA alleles.

5. The population of antigen-specific T cells of any one of claims 1-4, wherein the HLA type of each donor differs from at least one other donor on one or more class I HLA allele.

6. The population of antigen-specific T cells of claim 5, wherein the HLA type of each donor differs from the HLA type of at least one other donor on two or more class I HLA alleles.

7. The population of antigen-specific T cells of claim 6, wherein the HLA type of each donor differs from at least one other donor on at least one HLA-A and one HLA-B allele.

8. The population of antigen-specific T cells of claim 1, wherein the HLA type of each donor differs from at least one other donor on one or more Class II HLA alleles.

9. The population of antigen-specific T cells of claim 8, wherein the HLA type of each donor differs from at least one other donor on two or more alleles independently selected from the group consisting of HLA-DPA1, HLA-DPB1, HLA-DQA1, HLA-DQB1, HLA-DRA, and HLA-DRB1.

10. The population of antigen-specific T cells of claim 9, wherein the HLA type of each donor differs from at least one other donor on at least one HLA-DRB1 allele and one HLA-DQB1 allele.

11. The population of antigen-specific T cells of any one of claims 1-10, wherein the plurality of donors have at least 2 different HLA-A alleles, at least 2 different HLA-B alleles, at least 2 different DRB1 alleles, and/or at least 2 different DQB1 alleles.

12. The population of antigen-specific T cells of any one of claims 1-11, wherein the plurality of antigen-specific T cell lines are derived from 3 or more different donors.

13. The population of antigen-specific T cells of any one of claims 1-12, wherein the plurality of antigen-specific T cell lines are derived from 5 or more different donors.

14. The population of antigen-specific T cells of claim 12 or 13, wherein the HLA type of each donor differs from at least two of the other donors on at least one HLA allele.

15. The population of antigen-specific T cells of any one of claims 1-14, wherein the HLA type of each donor differs from each other donor on at least one HLA allele.

16. The population of antigen-specific T cells of any one of claims 1-15, wherein at least one of the plurality of different donors match on at least two HLA alleles with the greatest possible number of patients in a prospective patient population.

17. The population of antigen-specific T cells of any one of claims 1-16, wherein at least one of the plurality of different donors match on at least four HLA alleles with the greatest possible number of patients in a prospective patient population.

18. The population of antigen-specific T cells of any one of claims 1-16, wherein the population comprises T cells that match on each HLA allele with one or more patients in a prospective patient population.

19. The population of antigen-specific T cells of any one of claims 1-18, wherein the plurality of antigen-specific T cell lines are derived from 15 or fewer donors, 10 or fewer donors, or 5 or fewer donors.

20. The population of antigen-specific T cells of any one of claims 1-19, wherein the antigen-specific T cell lines from each donor are pooled together after each cell line is generated.

21. The population of antigen-specific T cells of any one of claims 1-20, wherein the antigen-specific T cell lines from each donor are pooled together after each cell line is individually assessed for cell line identity, viability, sterility, phenotype, potency, and/or alloreactivity.

22. The population of antigen-specific T cells of claim 21, wherein the potency of each cell line is assessed by IFNγ production.

23. the population of antigen-specific T cells of claim 22, wherein IFNy production is determined by IFNγ ELISPOT assay.

24. The population of antigen-specific T cells of claim 21, wherein the sterility of each cell line is determined by testing for bacterial contamination, fungal contamination, mycoplasma, and/or endotoxin levels.

25. The population of antigen-specific T cells of claim 24, wherein each cell line has an endotoxin level of less than 5 EU/mL.

26. The population of antigen-specific T cells of claim 21, wherein the phenotype of each cell line is assessed by flow cytometry.

27. The population of antigen-specific T cells of claim 21, wherein each cell line comprises at least 90% CD3+ cells.

28. The population of antigen-specific T cells of claim 21, wherein the alloreactivity of each antigen-specific T cell line against unrelated and/or partially HLA matched and/or HLA unmatched target cells is assessed by chromium release assay.

29. The population of antigen-specific T cells of claim 20, wherein the pool of cell lines is HLA typed.

30. The population of antigen-specific T cells of claim 20, wherein the pool of cell lines is tested for functional responses using HLA-restricted epitopes.

31. The population of antigen-specific T cells of any one of claims 1-30, wherein the antigen-specific T cell lines from each donor are pooled together after each cell line has been individually cryopreserved and then subsequently thawed.

32. The population of antigen-specific T cells of any one of claims 20-31, wherein the pooled antigen-specific T cell lines are cryopreserved.

33. The population of antigen-specific T cells of any one of claims 20-31, wherein the T cell lines are pooled together at a ratio of about 1:1.

34. The population of antigen-specific T cells of any one of claims 1-32, comprising from about 10×10⁶ to about 100×10⁶ T cells.

35. The population of antigen-specific T cells of claim 34, comprising about 45×10⁶ T cells.

36. The population of antigen-specific T cells of any one of claims 1-35, wherein the T cells are specific for one or more viral antigens or one or more tumor associated antigens.

37. The population of antigen-specific T cells of claim 36, wherein the one or more viral antigens are from one or more viruses selected from the group consisting of Epstein Barr virus (EBV), cytomegalovirus (CMV), Adenovirus (AdV), BK virus (BKV), JC virus, human herpesvirus 6 (HHV6), respiratory syncytial virus (RSV), influenza, parainfluenza, bocavirus, coronavirus, lymphocytic choriomeningitis virus (LCMV), mumps, measles, human metapneumovirus (hMPV), parvovirus B, rotavirus, merkel cell virus, herpes simplex virus (HSV), hepatitis B virus (HBV), hepatitis C virus (HCV), hepatitis D virus (HDV), human papilloma virus (HPV), human immunodeficiency virus (HIV), human T-cell leukemia virus type 1 (HTLV1), human herpesvirus 8 (HHV8), West Nile virus, zika virus, and ebola virus.

38. The population of antigen-specific T cells of claim 36 or 37, wherein the one or more viral antigens comprise antigens from BKV, CMV, AdV, EBV, and HHV-6.

39. The population of antigen-specific T cells of claim 36 or 37, wherein the one or more viral antigens comprise antigens from RSV, influenza, parainfluenza, and hMPV.

40. The population of antigen-specific T cells of claim 36, 37, or 38, wherein the one or more viral antigens comprise antigens from a coronavirus.

41. The population of antigen-specific T cells of claim 40, wherein the coronavirus is SARS-Cov-2.

42. The population of antigen-specific T cells of claim 36 or 37, wherein the one or more viral antigens comprise antigens from HBV.

43. The population of antigen-specific T cells of claim 36 or 37, wherein the one or more viral antigens comprise antigens from HHV-8.

44. The population of antigen-specific T cells of claim 36, wherein the one or more tumor associated antigens are selected from the group consisting of CEA, MHC, CTLA-4, gp100, mesothelin, PD-L1, TRP1, CD40, EGFP, Her2, TCR alpha, trp2, TCR, MUC1, cdr2, ras, 4-1BB, CT26, GITR, OX40, TGF-α. WT1, MUC1, LMP2, HPV E6 E7, EGFRvIII, HER-2/neu, MAGE A3, p53 nonmutant, NY-ESO-1, PSMA, GD2, Melan A/MART1, Ras mutant, gp 100, p53 mutant, Proteinase3 (PR1), bcr-abl, Tyrosinase, Survivin, PSA, hTERT, EphA2, PAP, ML-IAP, AFP, EpCAM, ERG (TMPRSS2 ETS fusion gene), NA17, PAX3, ALK, Androgen receptor, Cyclin B1, Polysialic acid, MYCN, RhoC, TRP-2, GD3, Fucosyl GM1, Mesothelin, PSCA, MAGE A1, sLe(a), CYP1B1, PLAC1, GM3, BORIS, Tn, GloboH, ETV6-AML, NY-BR-1, RGS5, SART3, STn, Carbonic anhydrase IX, PAX5, OY-TES1, Sperm protein 17, LCK, HMWMAA, AKAP-4, SSX2, XAGE 1, B7H3, Legumain, Tie 2, Page4, VEGFR2, MAD-CT-1, FAP, PDGFR-β, MAD-CT-2, and Fos-related antigen1.

45. The population of antigen-specific T cells of any one of claims 1-44, wherein one or more T cells in the population expresses an exogenous molecule.

46. The population of antigen-specific T cells of claim 45, wherein the exogenous molecule is a therapeutic agent.

47. The population of antigen-specific T cells of claim 46, wherein the therapeutic agent is a chemotherapeutic drug, cytokine, chemokine, small molecule inhibitor of tumor growth, or a molecule that sequesters immune inhibitor molecules.

48. The population of antigen-specific T cells of any one of claims 45-47, wherein the exogenous molecule is a transgenic molecule.

49. The population of antigen-specific T cells of claim 48, wherein the transgenic molecule comprises an extracellular binding domain, a transmembrane domain, and a signaling domain.

50. The population of antigen-specific T cells of claim 49, wherein the extracellular binding domain is specific for a cancer antigen.

51. The population of antigen-specific T cells of claim 48, wherein the transgenic molecule is a chimeric antigen receptor (CAR), a T cell receptor (TCR), or an NK cell receptor.

52. A composition comprising the population of antigen-specific T cell lines of any one of claims 1-51.

53. The composition of claim 52, comprising a cryopreservation media.

54. The composition of claim 53, wherein the cryopreservation media comprises human serum albumin, Hank's balanced salt solution (HBSS), and about 10% (vv) dimethyl sulfoxide (DMSO).

55. The composition of claim 54, wherein the cryopreservation media comprises about 50% (v/v) of 25% human serum albumin and about 40% (v/v) HBSS.

56. A universal antigen specific T cell therapy product comprising the population of antigen-specific T cells of any one of claims 1-51, wherein the product exhibits a lack of alloreactivity to partially HLA-matched and/or to HLA mismatched target cells.

57. The universal antigen specific T cell therapy product of claim 56, wherein the plurality of antigen-specific T cell lines comprise sufficient HLA diversity with respect to one another that they collectively provide at least one antigen specific T cell line that is matched on at least 2 HLA alleles with >95% of a prospective patient population.

58. A method for treating a disease or condition comprising administering to a patient in need thereof a population of antigen-specific T cell lines of any one of claims 1-51, a composition of any one of claims 52-55, or a universal antigen specific T cell therapy product of claim 56 or 57.

59. The method of claim 58, wherein the population, composition, or T cell therapy product comprises a mixture of T cells, wherein the mixture of T cells comprises T cells that are partially matched and T cells that are completely mismatched with the HLA type of the patient.

60. A method for treating a disease or condition comprising administering to a patient in need thereof a universal antigen specific T cell therapy, comprising administering to the subject a plurality of antigen-specific T cell lines from a plurality of different donors, wherein the HLA type of each donor differs from at least one of the other donors on at least on HLA allele, and wherein the method comprises administering the plurality of antigen-specific T cell lines to the patient in a single dosing session.

61. The method of claim 60, wherein administering in a single dosing session comprises administering the plurality of antigen-specific T cell lines to the patient simultaneously in the same composition.

62. The method of claim 60 wherein administering in a single dosing session comprises administering the plurality of antigen-specific T cell lines to the patient in separate compositions administered sequentially.

63. The method of claim 62, wherein the sequential administrations are performed within 1 hour of one another.

64. The method of any one of claims 58-63, wherein the population or plurality of antigen-specific T cells comprise a mixture of T cells comprising T cells that are partially matched with the HLA type of the patient and T cells that are completely mismatched with the HLA type of the patient.

65. The method of any one of claims 58-64, comprising administering to the patient a dose of about 10×10⁶ to about 100×10⁶ antigen-specific T cells.

66. The method of claim 65, comprising administering to the patient a dose of about 45×10⁶ T cells.

67. The method of any one of claims 58-66, wherein the disease is a viral infection.

68. The method of claim 67, wherein the antigen-specific T cells are virus-specific T cells (VSTs), and wherein the method achieves a reduction in viral load in the patient and/or reduction or elimination of symptoms of a disease associated with the viral infection.

69. The method of claim 67, wherein the antigen-specific T cells are VSTs, and wherein the method achieves a faster resolution of viral infection relative to a patient that did not receive the VSTs.

70. The method of any one of claims 58-69, wherein patient is immunocompromised.

71. The method of claim 70, wherein the patient is immunocompromised due to a treatment the patient received to treat the disease or condition or another disease or condition.

72. The method of claim 70, wherein the patient is immunocompromised due to age.

73. The method of claim 72, wherein the patent is immunocompromised due to young age or old age.

74. The method of claim 71, wherein the condition is an immune deficiency.

75. The method of claim 74, wherein the immune deficiency is primary immune deficiency.

76. The method of claim 71 wherein the patient is in need of a transplant.

77. The method of claim 71, wherein the disease is a cancer.

78. The method of claim 77, wherein the cancer is selected from the group consisting of lung cancer, bowel cancer, colon cancer, rectal cancer, bile duct cancer, pancreatic cancer, testicular cancer, prostate cancer, ovarian cancer, breast cancer, melanoma, soft tissue sarcoma, lymphoma, leukemia, and multiple myeloma.

79. A method for generating a universal antigen specific T cell therapy product comprising a population of antigen-specific T cells, the method comprising
(i) culturing mononuclear cells from each donor of a plurality of donors in the presence of one or more cytokines and one or more antigen, to generate a plurality of individual cell lines of expanded antigen-specific T cells, and
(ii) pooling together the individual cell lines to generate the universal antigen specific T cell therapy product.

80. The method of claim 79, wherein the mononuclear cells are peripheral blood mononuclear cells (PBMC).

81. The method of claim 79, wherein the population is a clonal, oligoclonal, or polyclonal population.

82. The method of claim 79, further comprising a freeze-thaw, wherein each cell line is cryopreserved and then thawed prior to the pooling of (ii).

83. The method of any one of claims 79-82, further comprising freezing the pool of cell lines obtained in (ii).

84. The method of claim 83, wherein the pool of cell lines is cryopreserved as a universal antigen-specific T cell therapy product.

85. The method of any one of claims 82-84, wherein the cell lines or universal antigen-specific T cell therapy product are cryopreserved in cryopreservation medium.

86. The method of claim 85, wherein the cryopreservation medium comprises human serum albumin, Hank's balanced salt solution (HBSS), and about 10% (vv) dimethyl sulfoxide (DMSO).

87. The method of claim 86, wherein the cryopreservation medium comprises about 50% (v/v) of 25% human serum albumin and about 40% (v/v) HBSS.

88. The method of claim 79, further comprising a filtration step.

89. The method of claim 88, wherein the method comprises filtering each cell line obtained in (i).

90. The method of claim 88, wherein the method comprises filtering the pooled universal antigen specific T cell therapy product obtained in (ii).

91. The method of claim 88, wherein the method comprises filtering each cell line and/or filtering the pooled universal antigen specific T cell therapy product, before and/or after a freeze-thaw step.

92. The method of claim 79, further comprising transfecting one or more individual cell line obtained in (i) with a transgene.

93. The method of claim 79, further comprising transfecting the pooled cell lines obtained in (ii) with a transgene.

94. The method of claim 93, wherein the transgene encodes a chimeric antigen receptor (CAR), a T cell receptor (TCR), or an NK cell receptor.

95. The method of claim 79, wherein the culturing is in a vessel comprising a gas permeable culture surface.

96. The method of claim 95, wherein the vessel is a GRex bioreactor.

97. The method of claim 79, wherein the one or more cytokines is IL4 and/or IL7.

98. The method of claim 97, wherein the cytokines comprise IL4 and IL7 and do not comprise IL2.

99. The method of claim 79, wherein the one or more antigen is in the form of 100. (i) a whole protein, (ii) a pepmix comprising a series of overlapping peptides spanning part of or the entire sequence of each antigen, or (iii) a combination of (i) and (ii).

101. The method of claim 99, wherein the antigens comprise at least 2, 3, 4, 5, 6, 7, 8, 9, 10, 11, 12, 13, 14, 15, 16, 17, 18, 19, 20, or more different pepmixes.

102. The method of claim 79, wherein the one or more antigens are viral antigens or tumor associated antigens.

103. The method of claim 101, wherein each antigen in the culture is a viral antigen.

104. The method of claim 102, wherein the viral antigens are from a virus selected from EBV, CMV, Adenovirus, BK, JC virus, HHV6, RSV, influenza, parainfluenza, bocavirus, coronavirus, LCMV, mumps, measles, human metapneumovirus, parvovirus B, rotavirus, merkel cell virus, HSV, HBV, HCV, HDV, HPV, HIV, HTLV1, HHV8, West Nile virus, zika virus, and ebola virus.

105. The method of claim 101, wherein each antigen in the culture is a tumor associated antigen.

106. The method of claim 104, wherein the tumor associated antigens are one or more of CEA, MHC, CTLA-4, gp100, mesothelin, PD-L1, TRP1, CD40, EGFP, Her2, TCR alpha, trp2, TCR, MUC1, cdr2, ras, 4-1BB, CT26, GITR, OX40, TGF-α. WT1, MUC1, LMP2, HPV E6 E7, EGFRvIII, HER-2/neu, MAGE A3, p53 nonmutant, NY-ESO-1, PSMA, GD2, Melan A/MART1, Ras mutant, gp 100, p53 mutant, Proteinase3 (PR1), bcr-abl, Tyrosinase, Survivin, PSA, hTERT, EphA2, PAP, ML-IAP, AFP, EpCAM, ERG (TMPRSS2 ETS fusion gene), NA17, PAX3, ALK, Androgen receptor, Cyclin B1, Polysialic acid, MYCN, RhoC, TRP-2, GD3, Fucosyl GM1, Mesothelin, PSCA, MAGE A1, sLe(a), CYP1B1, PLAC1, GM3, BORIS, Tn, GloboH, ETV6-AML, NY-BR-1, RGS5, SART3, STn, Carbonic anhydrase IX, PAX5, OY-TES1, Sperm protein 17, LCK, HMWMAA, AKAP-4, SSX2, XAGE 1, B7H3, Legumain, Tie 2, Page4, VEGFR2, MAD-CT-1, FAP, PDGFR-β, MAD-CT-2, and Fos-related antigen1.

107. The method of any one of claims 79-105, wherein the plurality of donors are selected by a method comprising:
(a) comparing an HLA type of each of a first plurality of potential donors from a first donor pool with each of a first plurality of prospective patients from a first prospective patient population;
(b) determining, based on the comparison in step (a), a first greatest matched donor, defined as the donor from the first donor pool that has 2 or more HLA allele matches with the greatest number of patients in the first plurality of prospective patients;
(c) selecting the first greatest matched donor for inclusion in the universal antigen specific T cell therapy product;
(d) removing from the first donor pool the first greatest matched donor thereby generating a second donor pool consisting of each of the first plurality of potential donors from the first donor pool except for the first greatest matched donor;
(e) removing from the first plurality of prospective patients each prospective patient that has 2 or more allele matches with the first greatest matched donor, thereby generating a second plurality of prospective patients consisting of each of the first plurality of prospective patients except for each prospective patient that has 2 or more allele matches with the first greatest matched donor; and
(f) repeating steps (a) through (e) one or more additional times with all donors and prospective patients that have not already been removed in accordance with steps (d) and (e), wherein each time an additional greatest matched donor is selected in accordance with step (c) that additional greatest matched donor is removed from their respective donor pool in accordance with step (d); and each time a subsequent greatest matched donor is removed from their respective donor pool, each prospective patient that has 2 or more allele matches with that subsequent greatest matched donor is removed from their respective plurality of prospective patients in accordance with step (e); thereby sequentially increasing the number of selected greatest matched donors for inclusion in the universal antigen specific T cell therapy product by 1 following each cycle of the method and thereby depleting the number of the plurality of prospective patients in the patient population following each cycle of the method in accordance with their HLA matching to the selected greatest matched donors; wherein steps (a) through (e) are repeated until a desired percentage of the first prospective patient population remains in the plurality of prospective patients or until no donors remain in the donor pool.

108. A universal antigen specific T cell therapy product produced by a method according to any one of claims 79-106.

109. A composition comprising a universal antigen specific T cell therapy product of claim 10.
